SEQUENCE LISTING (18 sequences)

SEQ ID NO: 1 - DNA, 31790 bp, Homo sapiens
gggaagctccaggcaaacagcccagcaaacagcagcactcagctaaaaggaagactcacagaacacagttgaagaaggaaagtggcgatggacctcatcccaaatttggcggtggaaacctggcttctcctggctgtcagcctggtgctcctctatctgtgagtaactgtccaaactcctctctttgtttccttggacttggggtgctaatcgggccccttttcccttatctgttttgaagatcaaaagagatgttcaaggagaagtagctgaagtgttggacgctacaaacgcatagaagttattattatcttatgcagatctatgaatgaataaataagcatttctcccatccaccttctaattttggtgactaggagggtttagggacagcatttggtagtgggaatgatttgattagcttagatctgacgaagactaatcaatgaaaacatggcagcggcagattacaaactgctgatcatgatggacagtgtgatcctcatccccttcccaggctctggggattctgggtacaggaaggagtggcttgcatttttgtctcattaattcgctttctgggttctgtgtctgctggaagggatgtgtagctgtattgcccctgtagacctggttcctgctcccccgccttccaacccaggatatcatttacataacgcaccaggggacaccaagacttcatgggaagctgtcccctggctcttccctctttcctgtgccatgcccctgaaaatcccctccctcctatgagtcactcctccaccctgtcatacacaggatggtttatcttgcaatgattaacctctagagcaaaggagacctggaggaagtttcgaggatttattctttgctttaatctttttcctcccgtctctgggaggctaggattaatatagagctttgtttctcacctaatgggaatctactagcagcctgaaaaggcaggagccatgaaagccaatttggattttacatatttttcccctttatgttacagtacaggagggcaaaccctctcactggtgggattcctggcatcctagagcaggtggagagaagagttactttccactgtgggtagtggaggctccacctgtcccattaacttctacctcaatttgacttttattaagagcagggaaccacaatgacatgaaaatagacactataaacctcattttaattctttcacagaaagcttaggaattcagtgagttgtggcaacatggtttccattgtctaacatttttaaatgaattgatatggtttaaattcattcatttttaaaccagaattttttggagatagactatttccagcatgttccttctggatggtaaaacagggctgttagttcagtatttgtgacaataagtgtgtgtaaaataatgtcacctttcctgaatgtcaggaatatgagtctaatgcacaaatgtatacctctaagacaagactgcacgtcttttcaaatatacctgtccggccatttattttaataactccttttcgaatatacctgcttagcagattgtcttaaactctcaggacaggggagtaagcaagactgtgagccagtgacgatagcaaaggcttccaggtaggatccatatgaagtgagaaaatattcctcagctctcagggtagaactccaaagagatattcatgggtcctggccccaccgtggaggtcactcaaagggcaaacaggttggcatctcatctgcttcaagcctggacacaggggcaccatctgtgtcactctgtgtgtggtctgccatgttgtgggccggtcactacagactcgggcagccaggcagacaatgccttagccttagacaatgctggtgcagcccaggagtcagaaaatgcagtgtagaccaggccctccttaggccaacacaattacatgcaatagatgactggcttttctgttagtctcttcactggacccaaaggctgcattactctaccagaggggagctggaaagaaactaaagagttcgcccagcacagcatctgccttgacatggtaccatgtgaatctagacactcaccaagatctttccttgggggccaatgctgctgacacattaactcaatagcttgtcctcacctgagaggtcaggtaatgtgtttaaagttcaggagcagagattagtgtcattgatttgacatggctgtgacaacaaaggagggaactgaagtgggaatacccaaggccaccctggctttggcaggtggtgcacgcacttccactaactgttctggggcagggaaccaaatgtatgactgggcctgctcatgctgcccctgctgagtcctccaaaccctgcccttcatgtaatttctcagttttattttatcacattttataagtcactggatgtttacaaaatgtttggaacctatactgccttgaaggctaacctctaaagaggagtaaacaaggtcttaatacaactctccgggacgttttatcattacttatcttatatgccatactgcaccatttgctatcaacaggaaagtacctggactttggaaggtccctctgtgtcttttagctgaaagtacatatgaggcatgtggattcttttatgcacatcatctttttcagccacatttttgtagtttgcctctctggagccaactgtgtggggctagcagcttcacagctgaatcagtgtctggcaacctcttccttcagcctctcttcttcctccagttttccatccctcagtcacaccggagggggaaggtctgcaaggatccagaaccatcagttggaggagtttgcacatgactcatgaaagatgagttccaggcaggcctgccatagtgaacaccaggcttaatgggtttttcctcagagatacttcacgtacagaggcagtgaactgactgctttctggttgaccaccttgaaaaagatgagtgtgcctggcactgtgcttctcaggtgagtatgacctgagaagtattagttgctggttcttctgcacacaatcattcaaggacatatggatcaaccatcctcctcaacagctcaaatcaaccagatcatctgaccacagagactgaggtgtacctgaaagctgcccacatttctataaggccaatagaagccatgaacacagttgtcaatctgtagaaataaggactccatgactcctccaaggcctctctgtgaatgaacgtttaagaagggctagatcctaaaacagggtcagagcttagagggaagaaaaagcataaacatttctgagcaaattgtaagggcagtgtcaccataggctcccagtgaccctctgtgattgagtgcatacagtgatgcaaaatctcatcatcagtgcaaaagacaaaaaaaatcttactctttctacctaggatgagagtccccaaatcagcgaagagtccacttactaaacagacataaggaaatgaagtgtcctggaagaattcctgcctgaacctctcaggagcatttgaggacatttatcaagtattcactccaggattgggactatgaagacttcagctgctttcagctaatcattgagacttttcaggggtctcagaatagtcaggaaaggacctgatgagtgaatgcaattactgatgttggagttgctgttattatttatcgtgtacatattacctccctctcttgaccattccagttcctgagtaactcaccagccctctgatctataaagtcacaatccctgtgacctgatttctgtttcactttgtagatatgggacccgtacacatggactttttaagagactgggaattccagggcccacacctctgcctttgttgggaaatgttttgtcctatcgtcaggtgagttgcttgagcttcctcttttgcttcttatggttgcaaacatcagcttagttccatcagtaaaaatgcccctccttgggagggagttctgaggtttcacattttcagaaatggtgggactgggtgcagtggatcatgcctgtaatctcagcctctgtgaggccaagactggcaaattgcttgagcccaggagtttgagaacagcctgggcaacacagtgagacacctgtctctagaaagaaaaaattacctgtgcatgatatggtagcccatgcctgtagtcccagctactctgaatgttaaggtgggaggattgtatgaacccaggaagtcaaggctgtattgagctgtgatcgcaccactgcactccagcttggtcaacagaacaagacagaaaggaagaaagaaagagagagagagaaagaaagagagaggaaggagaggggaggggaggggaggggaggggggaggagaggagaggagagaaaaggagaggagagaggagaggagaggaaaaggtgtgtaggctccacccaaagcatggccaggtttacccctggagggaaagtcacaagctcatgtccagaaggccagtagcagcaagctgctctccagcccagatttcctatcctgtgtacctggagcttgtttctcagattctaactctcacaactgaagcctctgttgtctgattactatctgagaattctacacaattttaccctcgataaaagcagtaatttcttcttcatctttcccagatcaactcttgtagtagatcaacatttctgggaccttcttttgcatggttaaaacatcacagctgaatcttagcaacaggaaggtttgtttttatgtttcagaagtgaaagctcagagcacgcattgtaatttgctgggtgtgatgtgtagaggtggcatttctccatcttttctgtgttaagctagaaaactggaaaggaagtctactttctcattcactcactcactttctcactcaacaacatgccttagacttatctaaatctgcaagactaaaagaggttcctggtttctttaactttctaattctgctagagttctagagagagcacatgagataaatgaaaaggatactgatggaggagattaaaaaattgtgcattccctgcagacactcacttttcctcacctcagtttcacccctgcccttgcaggtgatcattcacggggttaggagactttagagagaataaaagaaaaagcaaaaatacatcagaaagacaaggaattacttactggtcatagacaagggtgagtccttcagtacttagagaaaattcaagagtgactttaaattccccacttcaaatatattctctgttttcttgtctttcccttaagacatctctgaatagcttccttcaactgccagtgaaagatagcaggcctgatttcattggacgcaactgttttcagccccaattagaggtagggtttattctatttaaaataataatcaacttgtattttgtttcctctcccagggtctctggaaatttgacacagagtgctataaaaagtatggaaaaatgtgggggtgagtattctgaaaacctccattggatagacctgctactgtgaggaggttaccccactgcaggatagtctctgcccaggtcttcatgggatgaagctcttgtcaacctaaatacaaacagagagaggttctctgaaagaagaggataattacttgggagtagaatattgcaatgggaatctgcttgccgttataaactatgtgcaaattcagggaggtaaacaagacaaagatgctccatagaaaatatgagaagaatctcataactgttttgagataattattgttagctacaaagatcaataacaagggtgatgccacaccaaggttggacaggcagttgctggacaggtgtccttgcagaaatatttttgtgtaaagttgaaatagcctttgtgcaaagttgtggtttttgtagacacttttgtaatagttttgtttccaggaacacaagcataagaatcctctcttcatagccttcttgggatttatttgtcagggttaaaaaacaattagtgacatcactttggttctgataaagttcacactcgctattgtaaaacttttcgaggcttgtcctaccaaggatcccatgtgtcaccaggtatcgaggtcttcagtctgaactaggctaggagcattgtggttaccacttttctgcaggttttggtggcccagggactcccagcatcgccttctgtccagtgtctgcctattcccctcttctttttttcttccttaggtgcccttttatcacatgcattgtctcagacccttctaatatgtgctcataaatgcatggcatcatctccttcccacattgattcactttcaattaaaagccaaaactccttcatttagactgaatttaacatgtgcttttgaaagaagggttgagagataatagagaaacagattgggaaaccacttatgctccacttttttaaactttctctgcaagtatggaattttttgttctgctttgttgtttaaatttaagccaaaacttcttaatagaaggatatacaaatatttattggtttataccattgcacttactttgaagaagagatgctgaatattattaaaccattgtgttccctggtgggctgatggactgtgattttataaggtggtctcagccaattgcagcagctgttccctgtcagaggggctagaggtttggtgagagcagtggatgaggtgcagtggtgtgtttgttcactagaagcaagtgggagaaagctttgcctctttgtacttcttcatcttctcccctcaagtcctcagaatccacagcgctgactgtggagtgctgtggagctggcatggcccatacaggcaacatgacttagtagacagatgacacagctctagatgtccatgggccccacaccaactgcccttgcagcatttagtccttgtgagcacttgatgatttacctgccttcaatttttcactgacctaatattctttttgataatgaagtattttaaacatataaaacattatggagagtggcataggagatacccacgtatgtaccacccagcttaacgaatgctctactgtcatttctaaccataatctctttaaagagctcttttgtctttcagtatctcttccctgtttggaccacattacccttcatcatatgaagccttgggtggctcctgtgtgagactcttgctgtgtgtcacaccctaatgaactagaacctaaggttgctgtgtgtcgtacaactaggggtatggattacataacataatgatcaaagtctggcttcctgggtgtggctccagctgcagaatcgggctagtgaagtttaatcagctccgttgtccccacacagaacgtatgaaggtcaactccctgtgctggccatcacagatcccgacgtgatcagaacagtgctagtgaaagaatgttattctgtcttcacaaatcgaagggtaagcatccattttttgaaatttaaataatgattgatccactgattaaatttttattttgaaaaaaacatatattcacagaaggttacctaaaaaatgtacaggaaggttccatgtactcttcatcctgtcccgcccagtggtaacatcttgcaatcttgtatattgcaatatatatctagtatattcatattatcaggttggcacaaaagttaaaatggcaaactacaggctgggcataatggctcatgcctgtaatcccagcactttgggaggccgaggcaggtggatcacgaggtcaggagttcgagatcagcctgaccaacatggtgaaaccccatctctactaaaaatacaaaaattagctgcgtgtggtggcatgcgcctgtagtcccagctactcagtagtctgagacaggagaatcgcttgaacctgggaggcggaggttgcagtgagccgagatcacgccattatactccagtctgggcaacccaatgagactccatctcaaacaacaacaacaacaacaacaacaaaaaccggcaaactgcaataacttttgcaccaacctaatactatagtacaggaaattgactttgatatagtttacagagcttttcagatttcaccagttttacatgcccttgtttgtgtgtgtttatgtgtgtgggtagttctaagcaatttttcacattcgtagatttgtgcaacgaccagcaccatcaagatgcagacccattccgtcaccatgtggctccctcctgctgtcctacagtcacaacatggagtttgtctttttctctgacaggttctatatcagagcaaacttttatttatttgaggaggccaatgtattaatatttccttttatggattgttcttttggtgttaagtctgaaaatcctttgcttagccctccttcctacattgctttttctaagagttatatagtttaacactttacaaaatgtaactctattacccattttgtgttaatatttgcataagttatgagatttagatcaaggttcattttctgtggactatggctgtccaaatgttccaacaccattttggaaaggtaggcatattgtcaaaactcagctgagtatattttgtgaatctatttcttattgtttactcctccactaataccacactgtggtgactctagtagctgtacagtaactcttaacatcatatagggcaattctttccactttattgatttatattttcagaatggctttagcttttcttgtcccttgcctttccataaaaattcagaataagcttgtaagtgtctacaaacaaacctgccataattttgataagaattaaagcagaggtgtccaatcttttggcttccctgggccacagtggaagaagaagtgtcgtgggccacacataaaatacacacacacacacacacacacacacacacacacacacacacacacaaatggtctgtgtatagttttcattatatatctaccaccacagataagcaaaaatgtccttgcataataatcctaattatgcactgccccattcagagggtctttcaaaatcattgaacaggttccaagtttgcaatcactgatacagaaaatgtacatatctagctaaacttcactacttttttgatattttttattataaaagaaaagagaacaacataaaactagtggggtacttgacattgtttttgagaaactaatccatcagtatctggcttgatggaagtagttgcaattctcagtgagttctcaaggtgctcatcagatattttggttctaattttactcttcgtgttcttcatccttgaaaatagtagctcacaaatgtaagtgctgccaaaaagcaatgacatgaacaaggtgtgattgtgaagcaagggatatttgtcattgggaagacaggtcttacaaaagtccagtaaagaggcaaaatcaaatttttctataagttgaacatcagattgcagctctaggcattccatttcaaaattgccaggtaacatatatatgtcgactgaaaatggagttgcaaatataccaaaatattgatgattttttcagaaatcttgaaatacctgttttcaaattcctgtatcaaattgaaaagcaaggctgcgtatttttggctgttcacaggaccatgtttagccaacatgtcgaaatgcataaaattgtttgccttaatttgagcttgccataatttcagtttcatatggaatgctgttatggtttgaaacattgtattgttaagttggttttcaacttgaagacacaggtttaactcacttaaatgggccgtcaaacccactaaaaatgctaaatctgtaagccagttttcattgtcaagttctggcaccaattttgtttgataccataaacagcttgatttcacatcacaaagcataaaatctttacattttgccttgacttaaccatcttacttctaaaaagtgaatgacttgctagagtcagcatccatacttttaaggaattcctgaaactagcgatgattcaattcctgggcccttgtgaaatttacagccttgatgacaatttgcatgacgttatctacttttaaagcttgtgcacatggattttcttgatgtattatgcaataatacttcatcaaatgtgagttttgtgtggcaactgcatcatctattaattgtacaagtccctctcttttacctaccatcgccagggcagcatctgtagctatatcacatatgtttacaaaggacaaagaaaattgctttaacatatttttcactgcttcatataaatctcttgatttagttgtgtcttttaatagcatggtgacatttcgatttcttcagtgacattatattcatcatcaatacctctaataaaaatagcaagttgtgccgtatctgtagtgtcagtgccttcatccatcaccaaagcataaaattttaaattagcagttttactctccaaacgtctttcaatagatttcccaatttctccaattctcctggctatagtctggtgagacaaactgattttagaaatatcagtttctcaaggcaaataatatctaccacatcttccagacattgcttaataaactcaccatcagtaaatggttttgatttttttgctattaaatttgctaccacataactaggttttaccttacgattgagtccgagttgtaactttttaaaaaatcttttttgttgaaaagacagactttttttcagttctgctattttgtccttacaacacatacacaccaaatttgtcagcacgtttttgcatataatgcctcttcaaattgtagtctttgaaaactggcacaaattccgtggaaattaagcagagtgcttcgctatttgcctcaacaagaaaaagtcatttgtccacttttcattgaacaatcttccttcatccataatttttgttttttagggttttctttttaagacattgtggaagccattctggaattaaaagcattataatagataagcaactatatttacttttattatggaaattaacagataggaaaatagaacagaaagcaaggtttaataatcaaataagaatacttacatgtcttctaaataatattaaacacctatcatctacaaaggtaggttgaaatattattgataattgctgggttttacttgccaaattgccacaaacacacctaatacctgacagtgtcaattcaactgtccgtgattagaagataacacactggaagtcgcacaccaccataaaactgaagccacacatgcgtacaaatggcgacagtgtctggtgtacagcagcgctctgccttgtccagaatacacacttgaattctttgtcacaattcacttcacgtggcactgcaatagcgtcctctcgctctttgttagttaattttaatggcttttaatttcttcttgctgaactgtttgcaattataatgcaaattatggatactagtccattatttgtggatgtgacatactctgattacccctttccattccattgttgtctacgaagttcacacttgagaatcacatagtcaaattacaaaattacaaaaaaaattgcaaaaaaactcaaaatgttttaagaaagtttccacatttgtattgggatacattcaaagccatcctggactgcatgaggcctgcaggccacaagttggacaagcttgaattaaaccaatagaacaatttgggtataatctatatctttactatgttcagcctttcatcccgtgaatatagtatgcctctccatttctttagcttttattactttcctcaacattttatagttttcagcatagaggtcctgtacatcttttgttagatttacaccagaaatatttcatttttgttggagtaactgtaaatgatactgtttttcttgtattttcagatattgattattgttacatagaaatgtgaataattttgtttgttgatcttgtatcctatagccttgcagaacttacctattcgttctagaaatttttttgtatattccttgacattttatacattgacaattatgtcacctgaaaatagagacaattctattatttcctttccaatctgtatgccttttatttctttttcttgtctagtgtattaagacatcaggtatgctctttagtaagaatgttgagagtgggcattttttagttcttcttgatcttggaaaaaccattcagtccttcatcattaaatgtgatttaactgaatgatttttttacagattgtctttatcaaatgaaggaactgtctctctcttcctagtttattgagattttatcatgacagctggaagtacacattttaaaacaaaacatagttgtggaagataagagaaagttccaagcatgctggcttgatagtccagccccaagttgggaaaagtaattatccctttctttttccttctatttatggaataaaaaattaagagaaaagaattttcaaggaaattgcattattccttcaaaacaggtttctagtctttaagtattacctacttttcaaaaaaaaatcaccacatcatggcatccctttttcaagttgcccatgctgtaggtgtattaaagacagagctggtctgaggcaacatacagtctgcccatctgtcaccaatccttttctactctgcacactcctggggaagggctaggtcttgttcctgtctattccactggaagaacagttccctaccacgtggagcatttgcaattaaaaggagactgagatatagaggcaggagaccacaccagatggctgggtctccccactcccacccccgccccacatacactcagaagaggctaggcatctaggatctccattgagcatcttgaatatggcttgccataatatcatatacagtcaataaatatttgttaaataaggatgcctcttcaatatattttgtgcaaccatgaagatcaccacaactaatgtgagaaaaaatgtttctgttgaactctagtctttaggcccagtgggatttatgaaaagtgccatctctttagctgaggatgaagaatggaagagaatacggtcattgctgtctccaaccttcaccagcggaaaactcaaggaggtatgaaaataagatgagtcttaattagaaatgtaaagaatgaatctggggacaggtagaaagtaagatcacagtccgtttccaaggggtagtccactgagttcgagcttcctaaaaatggtcttttatctttatgtacagaaaagacatcacaaaattcattacaaaatgtcacttactgctccatgctggagaaagccatatccttctgggacttgagtctgcacatttaactacaggtactgatctgttttgtgcttagatgttccccatcattgcccagtatggagatgtattggtgagaaacttgaggcgggaagcagagaaaggcaagcctgtcaccttgaaagagtaagtaggagcacagccatggggttctgagctgtcatgagcccttccagctgcctgccatggagtcgacagtcgcactgttgggttactccagtgaccagacaaaagcagggcagcgctgcaactccaaagagccacctaagagggagtggctcccatgaggcggcaagtcagcaagggaaaagggccttctctcctgtgcacaggagccaggatttacttatctgttaacttgtcaccataaatattctgggagattaaatacatactttagaaattaaaaaaacatgattgtatcaaagttttgagtgtagtggatatggaactgtgggtaagcaagcatttggtacttgttgccttgcattgggtaagatgggaaagttacaatggggaacttggaacaatttcaatcccttcatggtttttctgagaatatcagcaaactatgaactattaaaccttcccactacttccttttcctccaatctcaaaaaagaaagggtgctagaaatgctatgtgtagagcaagcctattatttgctgtctacaatggtatgtgcttcaattatgcaggaacgacaggtgtaatctgagcctgtcctgttcagacttgggacatgtggtcactcagttttgggttctccaaatcaatgttggagagatctattttttttaaccagaacattcttgattgtcacatcttacaaaaatgactctgctctcagcgcaacttcaggtcagaggagctggggatagtggggttttccagagcattagcagggagtgtagagaataaaggatgatatttctaggaactcagaacagggtgttactgttttgtaaagtgttgaagaggaattggctctgggcatagagtctgtagtcagacaacgccacctttcttgaatccactaggaagagttaattattctactcttgttctgctgaagcacagagcttacatatcttatatcatccacactcaacacatgctactgtagttgtctgataatgggtctctgtcttcctatgactgggctccttgacctcagaggtgagtctaactcagcttggtgtctccatcacccccagcatagggccagctccatcactggcaccagataaccaccttctgagggagtagatggaagatgattcagcagatagttctgaaagtctgtggctctttatgtgtcttgactggatatgtgggtttcttgctgcatgtatagtggaaggacggtaagaggtgctgattttaattttccatatctttctccactcagcatctttggggcctacagcatggatgtgattactggcacatcatttggagtgaacatcgactctctcaacaatccacaagacccctttgtggagagcactaagaagttcctaaaatttggtttcttagatccattatttctctcaataagtatgtgggctattatttctttctctctttttaaaaataactgctttcttgacatataattcacatatcgtataattcatccacttaaaaggtacaattccattgtttttaagataatcaaaaatatgtatgaccattactattgtaaactaaaatgtttttgtcaatctagagccctcacacactttagctgtcaacaccccaccacaaaccccactgccctaagcatccaataatcaactttctgcctctatagatttgcctattctggacacttcatagaaataatatcattgatttttctctgttgttttttattctctatttcatgagtttattttagtctgttattttctttcttttgctggctttaggtttcatttgctcttcttcttttagtgttttgtggtgtaaataattataatcaatttgagatattttcttcttttaaatttagatattacagctataaatttccctctgagcactggtttggctacatcctgtgttttggtacatcatgccttctttttgttcatctcaaaacaatttcttgttgcccttttgatttctgctttgactcactggtcacttaaaactgtattgtttaacttccacaaatgtatgagtttcccaaatttctttcccttattgatttctagttttattccatggaagttgatgtacatatgctgtgttaattctatcttgactatcatttcctgaacagcatgattaagttaagcagcagattatggtctacattaatccaaaaactctagtccaatagataaaggctaagaggtcagggaatttaattctattactttggtcactccaaagactcagaaggtgccattgatctcactgctgtagtggtgtttcctatgtatagacctgcccttgctcagtcgccggcctgaaagaagggcaaacatgataaaaggaatgggttccagttgagaatcatgatgttcttattcttattactggtagagaaaattataattgctccaggtaaagtttgcattttcaatgatttccttttgtttgttttgtttttcccacagtactctttccattccttaccccagtttttgaagcattaaatgtctctctgtttccaaaagataccataaattttttaagtaaatctgtaaacagaatgaagaaaagtcgcctcaacgacaaacaaaaggtaaaatctgatggtggttaaatgacgatgtttaggttttgataaatttagattttatacacatgatagagcatgtatctgtatttttaaaaataaagacagagaacttatgtttagaacaagagaagccatttggtagaaataaagaaggagattggggaaggagatgagaatgagtcagagagatagcatttaaaacttgaaatcaggcacaacaattagtatgtcatgatataaacagtattgagataaaattttaccacttctcttccctttaataaattgtcaaaggataaagtttcctgtttgaaaatatattttactggtattgtgctttcctcatatcacagattggtaaagaatcattttaagtccaagactcttattttacatattctgcaattaaaggtcctatgaggctacctgccgactgctgacatgtagtgtgtggtaaatgtgagtgtttcacagcctggagtgaacaggggtcttctctgagaattgaggttgcaaggctggctaactcagctttgccttcacgagccctagaggccagccgaaggatgtctgcaggtcagggagacaggaccaggtaacccagctgtcactgaagattatatagagtttgagaatgttggaatatttgaaaatgctcccccaaaaaagctgctgatgagttctggaaatgtcaggagattaatctatacggacactgctgaagaaaaaggtagaagaataaaagatccagtacttcttcctgggtaagcagttatgaccagagatggaaccggcaactctttggccagaaagctgtatccaaaagacagagaagatgagaaacagggagggcaaaggcgaaaaagcaattggacatgatagctagatttgtttcaggaaaacatcctgctttccaaggatttagatgaatgtttttgttcactggtgactcaggtaacacgtcttcaagaagccatagggaggttgagggagggaagtcaagaagggaggttgaggactgcacttttgatttacttctgacttcacgagtcactttctgccaaagaaatctctccttttgcttctagcaccgactagatttccttcagctgatgattgactcccagaattcgaaagaaactgagtcccacaaaggtaaccaaggagtgcttctgagggctactggcggggacactaagagggagggccttgttctgaaaatgtgcaggaagtattccaggaagatgagaatttttgccacatagcagaacaacacacatttagatgttataaatggtagctggaggcactttccagaagcccacaggtatagccatgttccaggctgaaagggcaaccctaagcaaacctagaatgcttggaggacagtcagtggtttgtggatcacctacatgagatcaaatgccagttctcagcctcctccagatccaccaagtgagaacctctacttggaaatttatatcaaacataccgatcaggaagcacactatcccagtaagggtgattttaactggcagtacttgaaagtgtgttcgcaaggttaatctactgcaaagttttatttttccctttgaaatgcataagtaactaatgggggacacctctgataccatgtaaatctacttcaatcttcagtcttgtatctactagttttatgacccatggatggttttaaccaaaaccattattactaagacagtggcaaaatgataaccatggtcaatttcaagctaccaagatttggcaaccatctcacaaaatttttgaatatttaacaattggttctagagagcaggactcagcagactccagtataccactttaaacatgtccatgtctacatctacttctgtctgtctatctatctgtcaatcatctatctgcctataatttatcaattaatcatctatctatctcaacaaaacttgctgtgataaagaaaatagtctatcatttcactgtttcatatagaaatcactagacacatatggctattgagtactggacatgtggccaatgccactgaagaacaatttttaagagtatttatttttaattgaataaaatttgaatttaaatagccacatgtggatagtggctaccagattggacagcagagctcccaactttaaaattacagttcaatttcaactcagtataatggggttcaatgtaactgagtaaaataattggatggttgaatttacccacagcagcatacagaaatattcactgataaatcagaactctgtagacctttctcacactcattttatattgtgtttggttgtgagttacatgattgctgcaggcaccatatttatttctgtgctccaggtctctaaaggtcctaatccagtcctgaccaaacagactagtgatggaccatcgtgagcttctctcaggagaaatatcaagagggaggccaacctgtaatcataagaacttctgctattttaatgccattcatcagactacagtcaatcaccatgcttctggctttttgtctatctctgctgtcttgtacatcctgagatagtccattctgagaactgtaccctagatcttgtattgcctgatgcctgtcaaagatgtaatccatgctgcttaagtgaggttgtgcacacaaatcaccatatctcctgcaagtttggattttgattcagtagttcgatggtggggtttgagattctgcatttctaataagctcccagatgtggctggtgctgctggtccatgaaacacactttgagtagcaagaggtgatctgtagctcagtattggtcctttaagttccctcaaacatatatagagaaaaggtcctaaatattgcaaattctctcaaagtttgtcaagctatattggaattctctcaaagtctgtcaagctctattgtagaaaatcaaatttttattgggaaaaagcctaccccatatttacttacagataaagtacttttaggatcattcaaggcacacacccataacactgagtatgtaagacagaaatgctctctctggaaattacagcagtgctggtgctgggatgccatgatgaggagtgtgtggcccacaatcatgtagaccttgggaaaacctggattaaaatgattttgcgtcatcctggccctgtataagatacatatcagaatgaaaaccactcccagtgtgactttgaattgcttttccattttttcttcttgggattagagagcttcacttagatttcatctaagctgtgatgttgtacgttgacctgatttacctaaaatgtctttcctctcctttcagctctgtctgatctggagctcgcagcccagtcaataatcttcatttttgctggctatgaaaccaccagcagtgttctttccttcactttatatgaactggccactcaccctgatgtccagcagaaactgcaaaaggagattgatgcagttttgcccaataaggtgaggggatgacccctggagatgaagggaagaggtgaagccttagcaaaaatgcctcctcaccactccccaggagaatttttataaaaagcataatcactgattccttcactgacataatgtaggaagcctctgaggagaaaaacaaagggagaaacatagagaacggttgctactggcagaagcataagatctttgtacaatattgctggccctggttcacctgtttactgttatcacaataatgctaagtaaaaaaaaaaaaaaaaaaaaaaaaaaaaaaaggagtgtggcgagaagatggccaaacaggaacagctccagtctacagctcccagcgtgagcaacacagaagacgaatgatttctgcatttccaactgaggtaccgggtgcatctcaatggggattgttggagagtgggtgcaggacagtgggtgcagtgcacccagcctgagccaaagcagggcgaggcatcacctcacctgggaagtgcaaggggtcagggaattccctttcctaggggtgacggacagcacctggaaaatcaggtcactcccaccctaatactgcgcttttctgatggtcttagcaaacggcacaccaggagattatatcccgcgcatggctcggagggtcctacgcccatggagcctcgctcattgctagcacagcagtctgagatcgaactgcaaggcagcagcaaggctgggggaggggcgcccgccattgctaaggcttgagtaggtaaacaaagctgccaggaagctcaaactgggtgaagcccaccgcagctcaaggaggtctgcctgcctctgtagactccacctctaggggcagagcatagccaaccaaaaggcagcagaaacctctgcagacttaaatgtccctgtctgacagctttgaagagagtagtggttctcccagcacacagctggagatctgagaacagacagactgcctcctcaagtgggtccctgacccccgagcagcctaactgggaggcaccccccagtaggggcagactgacacctcacacggccgggtactcctctgagacaaaacttccagaggaatgatcaggcagcagcatttgcgggtcaccaataccgctgttctgcagcctccactcctgatacccaggcaaacagggtctggagtggacctccggcaaactccaacagacctgcagctgaggatcctgactgtcagaaggaaaactaacaaacagaaaggacatccacaccaaaacccatctgtacatcaccatcatcaaagatcaaaggtagataaaaacacaaagatgggggaaaaacagcagaaaaactgaaaaatctaaaaatcagagcacctctcctcctccaaaggaacgcagctccgcaccagcaacggaaagctggatggagaatgactttgacgagttgagagaagaaggcttcagacgatcaaactactccgagctaaaggaggaagttcgaacccatggcaaagaagttaaaaaccttgaaaaaagattagacaaatggctaactagaataatcaatgcagagaagtccttaaaggacctgatggagctgaagaccatggcacgagaactacgtgatgaatgcacaagcctcagtagccaattcaatcaactggaagaaagggtatcagtgatggaagatcaaatgaatgaaatgaagaaagaagagaagtttagaagaaaaagaataaaaagaaaggaacaaagcctccaagaaatatgggactatgtgaaaagaccaaatctacgtctgattggtgtacctgaaagtgacggggagaatagaacgaagttggaaaacactctgcaggatattatccaggagaacttccccaatctagcaaggcaggccaacattcaaattcaggaaatacagagaacgccacaaagatactcctcgagaagagcaactccaagacacataattgtcagattcaccaaagttgaaatgaaggaaaaaatgttaagggcagccagagagaaaggtcgggttacccacaaacacaaacccatcagactaacagtggatctctcggcagaaactctacaagccagtagagagtgggggccaatattcaacattcttaaagaaaagaattttcaacccagaatttcatttccagccaaactaagcttcataagtgaaggagaaataaaatactttacagacaagcaaatgctgagagattttgtcaccaccaggcctgccctaaaagagctcttgaaggaagcactaaacatggaaaggaacaactggtaccagccactgcaaaaacatgccaaattgtaaagaccatcgaggctaaggagaaactgcatcaactaacgagcaaaataatcagctaacatcataatgacaggatcaaattcacatataaaaatattaaccttaaatgtaaacgggctaaatgctccaattaaaagacacagactggcaaactggatagagtcaagacccatcggtgtgctgtattcaggaaacccatctcacgtgcaaagtaacacataggctcaaaataaagggatggaggaagatctaccaagcaaatggacaacaaaaaaaggcaggggttgcaatcctactctctgataaaacaggctttaaaccaacaaagatcaaaagagacaaagaaggccattacataatggtaaagggatcaattcaacaagaagagctaactatcctaaatatatatgcacccaatacaggagcacccagattcatgaagcaagtctttagagacttacaaagagagttagactcccacacaataataatggaagactttaacaccacactgtcaacactagacagatcaacaggacagaaagttaagaaggatatccaggaattgaactcagctctgcacaaagtggacataatagacatctacagaactctccaccccaaatcaacagaatatacattcttttcagcaccacaccacacctattccaaaattaaccacatagttggaagtaaagcactcctcagcaaatgtaaaagaacagacattataacaaactgtctctcagaccacagtgcaatcaaactagaactcaggattcagaaactcactcaaaaccgctcaactacatggaaactgaacaacctgctcctgaatgactactgggtacataacgaaatgaaggcagaaataaagatgttctttgaaaccaacaagaacaaagacacaacataccagaatctctgggccacattcaaagcaatgtgtagagggaaatttatagcactaaatgcctacaagagaaagcaggaaagatctaacattgacaccctaacatcacaatgaaaagaactagagaagcaggagcaaacacattcaaaagatagcagaaggcaagaaataactaagatcagagcagaactgaaggaaacagagacacaaaaaaacccttcaaaaaaatcaatgaatccaggagctggttttttgaaaagatcaacaaaattgatagaatgctagcaagactaataaagaagaaaagagagaagaatcaaatagatgcaataaaaatgataaaggggatatcaccacccatcccacagaaatacaaactaccatcagagaatactataaacacctctatgcaaataaactagaaaatctagaagaaatggataaattcctcgacacatacactctcccaagactaaaccaggaagaagttgaaactctgaatagaccaataacaggttctgaaattgaggcaataattaatagcttaccaaccaaaaaaagtccaggaccagatggattcaccgccgaattctaccagaggtacaaggaggacctggtaccattctttctgaaactattccaatcaatagaaaaagagggaatcctccctaactcattttatgaggccagcatcatcctgataccaaagcctggcagagacacaaccaaaaaagagaattttagaccaatatccctgatgaacagtgatacaaaaatcctcaataaaatactggcaaaccgaatccagcagcacatcaaaaagcttatccaccatgatcaagtgggcttcatccctgggatgcaaggctggttcaacatacgcaaatcaataaacataatccagcatataaacagaaccaacgacaaaacccacatgattatctcaatagatgcagaaaaggcctttaacaaaattcaacagcccttcatgctaaaaactctgaataaattaggtattgatggaacctatctcaaaataataagagcaaatttatgacaaacccacagccaatatcatactgaatggacaaaaactggaatcattccctttgaaaactggcacaagacagggatgccctctctcaccactcctattcaacatagtgttggaagttctggccagggcaatcaggcaagagaaagaaataaagggtattcaattaggaaaagaggaagtcaaattgtccctgtttgcagatgacatgattgtatatctagaaaaccccatcgtctcagcccaaaatctccttaagctgataaacaacttcagcaaagtatcaggatacaaaatcaatgtgcaaaaatcacaaatattcttatacaccaataacagacaaacagagagccaaatcatgagtgaactcccattcacaattgcttcaaagacaataaaatacctaggaattcaacttacaagggatgtgaaggacctcttcaaggagaattacaaaccactgctcaatgaaataaaagaagatacaaacaaatggaacaacattccatgctcatgggtaggaagaatcaatatcatgaaaatggccatactgcccaaggtaatttatagattcagtgccatcgccatcaagctaccaatgactttcttcacagaactggaaaaaactactttaaagttcatatggaaccaaaaaagagcccgcattgccaagtcaatcctaagccaaaagaacaaagccggaggcatcatgctacctgacttcaaactatactacaaggctacagtaaccaaaacagcatggtactggtaccaaaacagagatattgatcaatggagcagaacagagccctgagaaagaatgccacatatctacaaccatctgatctttgacaaacctgacaaaaacaagcagtggggaaaggattccctatttaataaatggtgctgggaaaactggctagccatatatagaaagctgaaactggatcccttccttacaccttatacaaaaattaattcaagatggattaaagacttacatgttagacctaaaaccataaaaaccctagaagaaaacctaggcaatatcattcaatacagaggcatgggcaaggacttcatgtctaaaacaccaaaagcaatggcaacaaaagccaaaattgacaaatgggatctaatgaaactaaagagcttctgcacagcaaaagaaactaccatcagagtgaacaggcaaccgacagaatgggagaaaatttttgcaacctactcatctgacaaagggctaatatccagaatctacaatgatctcaaacaaatttacaagaaaaaaacacaaccccatcaacaagtgggggaaggatatgaacagacacttctcaaaagacatttatgcagccaatagacacatgaaaaaatgttcatcatcactggccatcaaagaaatgcaaatcaaaaccacaatgagataccatctcacgccagttagaatggcgatcattaaaaagtcaggaaacaacaggtgctggagaggatgtggagaaaacaggaacacttttacactgttggtgggactgtaaactagttcaaccattgtggaagtcagtgtggtgattcctcagggatctagaactagaaataccatttgacccagccatcccattactgggtatatacccaaaggattataaatcatcctgctataaacacacatgcacacttatgtttattgcagcactattcacaatagcaaagacttggaaccaacccaaatgtccaataatgatagactggattaagaaaatgtggcacatatacaccatggaatgctatgcagccataaaaaatgatgagttcatgtcctttgtagagacatggatgaagctggaaaccatcattctcagcaaactatggcaaggacaaaaaaccaaacactgtatgttctcactcgtaggtgggaattgaacaatgagaacacatggacacaggaaggggaatatcacacactggggcctgttttggggtgggaggagtggggagggatagcattaggagatataccgaatgttaaatgacgagttaatgggtgcagcacaccaacatggcataggtatacatatgtaacaaacctgcacgttgtgtacatgtaccctaaaacttaaagtataaaaaaaaaaattcaaaaacctcagtggcatctaatgagaagcatttattgctcacaagactggatagtgagttctgctgatactgactggactcactctggtctggctatggtctgaggtagcctggccctgggggcgcgatggaggctgactcagctctccccacacctgtctcatgttccagtcaggtagccactggccaagaagccaagctaggaaccagggtatctgactcctgagctaaactctaaccctctacaatactgcctcccaaatataacaccaagtgctaggtacatatcatccacagttttcagacttctgcccaaactgggattctttttagtgtgaagagacctggcctgtggggctgaccctggtgtggctgtgaggcagacacaaagggacatttacatccagtcctgaagattacagtccagccctgaagcaacaactaggaaactattccaaaaggaggggatggggctgagtgtggggttctattctcttcataactttaactagaactcaaattgtgtaccttggtagcatccaatcataaatttattttgtcgtatttgtgatagaaaggaacaagtttatccacaaatttatttatttatttatttatttatttatttatttgagacagggtctgactctacgacccaagctggagggcagtggtgcaatctcagctcactgcaaactctgcctcccaggctcaagccatcctcccgcctctgtctcctgagtagctggaactacaggcacacgccaccacacccagctagtttttgtattttttgtagagatgggttttcaccatgtttcccaagctggtctcaaactcctcaaaagagttaccaagcaggactctgcaaccaataatccttgtgtgaagaggatatttgctcttttccctgtttttctttcttggtacagatgtgtgacctctttttgaaaggtgatagtgactttggtgtattttatttggtggtaatggtcatagccccattaatcacatttcttcccatgagaaagaaaaaccactacatggtcatgctaaggatttcagtccctggggtgaggatggtcttgaatatctcctacattcataactcctccacacatctcagtaggtcactgagcacatcaatggacatgccagttattaaaatacttcacgaatactatgatcatttaccagtatgagttattctctggagcttctaatacttcaatagtactgcatggactcagttgagagttaattcaaaatctcagattatccaattctgtttctttccttccaggcaccacctacctatgatgccgtggtacagatggagtaccttgacatggtggtgaatgaaacactcagattattcccagttgctattagacttgagaggacttgcaagaaagatgttgaaatcaatggggtattcattcccaaagggtcaatggtggtgattccaacttatgctcttcaccatgacccaaagtactggacagagcctgaggagttccgccctgaaaggtacaagtctccagggaaatggagctcaccctgacccaggctggttcaagcatattctgcctctcttaatctacatgacaatcgtgtggttgtacaatcatttgcttgtaagtctttttatcacaaaaaagtgataattatcaaactttacaaaccacagactagaaaaaacgaaactacatccatccacagtcccagcacaagacaaagataatcaattatgtccctgtgggcatttttctacgcctatatagatttttaaaaattagaatggtatcactttttatttggtttgaattgctgcttacttgatttaacaggaaactatccactgacctatattactataaatatacatatatatgtatatatataaatatatatatatgtatatattgcatatgccataaaccatttaaccatgatgttatttcaggtgtataggctttttattcctttctgttttttctatgctgtgccctttagctctctgaatttaacagaaactttaaaacatgcttccacattccatttgctttcaacgttacttgctatttcctctgtagtaattataagagtgcaggctgaggtcctgagaagtcctcatccctaatggtttaagccacttcactgaagacacaagacagcacaggtcctcctggtcctatctgtggctgcagtcctgtgccagctcccttatactctcagtagacatctcacacactcctccttggaggtgtcttgagcatgctcttctgggaattcagggacaaggtcaggccttaggcacagttcgcactctggatatagttggtgttttcccattactgtattattaagcaaaatttagaatgaaatttttagggtactggctggtgattcaggatgcttgggatctagactttcattagcccctacctgcaagtttgctgatgggaggaaccttgtcttgttggtcatggtgtccctagtgctagcatggagtctgcacataatacttgttcacagagtaagtcagagctgaccaagttctctgttttctggagtagaggacttctatgtttcctgcaagctcagcacttccacctcctgtggctgcactaatacgaaatcagagaccactcgctgtacttcactttgaatcactcagtcaccaaaaagatagtgcttgccatgtgtcaggaacttggctaggcagggagaaattcatatgatttatataaatccataaatccatatgatttacataaatccataaattcatgtgatatatacgtatatgtgtgtgtatatatatattagagaatgtttgacatatacacaagtacatgttaccgacaccagcctatagaatagttttcgtgcatctccatatatctatcactggttccaacagccatcaatccatgttagctgccccatccaaatgccaccatcaccctcctcctgactatcatgttattttgaagcaatagcctgtaaatatttcagaatgctctccaaaatataaagactcctgtaaaaacatatgacaacaatgccattattactttctttgaatcaacattttttccttaatataatcaaatatttagaaatcaaatttgaataaaacatgggtcaatcttcaaagaatttatagcttaatggaacagatcaaggaaagcagggatgacactacagtagggtagcatcatatgcccatgtaacttatgtgacttaaactatcctgtaagggtgtgggggagaaagagaggaagagatggagagaagaaaaaggaagagaaggaggaggagaaggaggcagaggagaaggtggacggggaaggtagagaggaggaggaggggaattagaaaaaaagagatgacaggagaaggaaagggaaaaataacaacttgaaatagcacaagacgttttctccttctcctttctcaatgagcatgtgaccaacacaagtgtgagttgaggcaggaatccacttttccatccatcagtcttatcatttatgtgccttttatagtgtgaacacatcaccaccctgaatataattttagtgtttagagataaatattatttgcaacaatattcatctcatctcaagaaacgctcctatagggtatggagaatttaaaggacctgtaggttatgatgattataacgaaataaccaaagcaggatttcaatgaccagcccacaaaagtatcctgtgtactactggttgggaggtggaggggggttgttcttaagtaagaacccctaacatgtaactctgtggtttttatgtttcattaactatttaatctaccaatatggaactaggttcagtaagaagaaggacagcatagatccttacatatacacaccctttggaactggacccagaaactgcattggcatgaggtttgctctcatgaacatgaaacttgctctaatcagagtccttcagaacttctccttcaaaccttgtaaagaaacacaggtcagtacactttctgtatgttttattaagaatttttttaactgaagggtatatattttttaaaagaatatgcatgtttatcttttaataattcattctatgggccaaagaacctacttggatccatctttgatcattaaggatgcttcagttctggacttcaaaacctgtagcattaagaacatcatgtaaagtccacacagattagcatgacatgattatgtgtagtctctttgaacctgagtaagtttaaattcagtttcaagtcaattggaaagaagtgttttgcacaatcatgaagtgcaatgattacctggctgtgacttaaatggtgttctccatcaccagaacctgcagaagctctctcatgacagtggttctcaaccactagctgtatattggaatcaccagggagcttcaaaaattcatgatgcctgtgacatctcagaaattctaaactaattaacccagagcgtgactaggttctgtcatgctgtcgggtgaacccctgattagttctcacgtgaagccaaggtggagaatgactaatttcaggcatttctggtggatatgaaggactaccatagagcagggctatccttactccttgaccttatgttccaggtgatacatttaaagaaagatttagaatcttttctctgaagaagttaaagaacagatgtcattgattcatattaagcaatagcctataagtcttatttccaggaccggtgtatttaatatgcaactctaccccttaagtacactttgtgcttgggagaggaggaggatggagatggttgccatcttatctatggcttcagggcagctgtgtagctttcctatgtgtgtattcaggcagggggctcagccctgagagaaagtgggcctctggcacacctgggacagggaagatattccctggcaagctctcaggcatctcaggctggcacttctttgtatccatggcaatttgctttcccctcactgaactgagatcagaatgttactctgttggtggctcccccaacagtgaaggggtgactcagtgacaatagtgctagaagtatgagtcaaaacactgtacaacttgagaaattccccgtttgcactacgcttggaagccaagaggagatgttaaaaagaaaagaataattctttctgaagacatttcccatcattgcacttgatgggttcaactgggaagggttactagactctggaagttgaaaactgcccacataattaaactgtacaacagctactcaggattaccttgcaagttttaacctataaaaatttaactttatatagcacttccaaaatagtttgccataatacctactaatctggatttaatttttaaaactcatccttttaacttaagatttaaataaaaaaaaaaaaacacgagtccacaagaatttgtctcaggcctggcacagagtcagtgctccataaatattttgttaaacgatggatggtgagtgcttttactatccagtatttacccagcttatagattaagtatgaagagttcaagatacatggtgttaagagtcgtttttatatgcttgcaaagcatttttgtcatattttttctactttgcttccatcttttcttctttcacttcatttattaattctccatatgcttgtttaactattgtagatccccttgaaattagacacgcaaggacttcttcaaccagaaaaacccattgttctaaaggtggattcaagagatggaaccctaagtggagaatgagttattctaaggatttctactttggtcttcaagaaagctgtgccccagaacaccagagatttcaacttagtcaataaaaccttgaaataaagatgggcttaatctaatgta

SEQ ID NO: 2 - DNA, 31790 bp, Homo sapiens
gggaagctccaggcaaacagcccagcaaacagcagcactcagctaaaaggaagactcacagaacacagttgaagaaggaaagtggcgatggacctcatcccaaatttggcggtggaaacctggcttctcctggctgtcagcctggtgctcctctatctgtgagtaactgtccaaactcctctctttgtttccttggacttggggtgctaatcgggccccttttcccttatctgttttgaagatcaaaagagatgttcaaggagaagtagctgaagtgttggacgctacaaacgcatagaagttattattatcttatgcagatctatgaatgaataaataagcatttctcccatccaccttctaattttggtgactaggagggtttagggacagcatttggtagtgggaatgatttgattagcttagatctgacgaagactaatcaatgaaaacatggcagcggcagattacaaactgctgatcatgatggacagtgtgatcctcatccccttcccaggctctggggattctgggtacaggaaggagtggcttgcatttttgtctcattaattcgctttctgggttctgtgtctgctggaagggatgtgtagctgtattgcccctgtagacctggttcctgctcccccgccttccaacccaggatatcatttacataacgcaccaggggacaccaagacttcatgggaagctgtcccctggctcttccctctttcctgtgccatgcccctgaaaatcccctccctcctatgagtcactcctccaccctgtcatacacaggatggtttatcttgcaatgattaacctctagagcaaaggagacctggaggaagtttcgaggatttattctttgctttaatctttttcctcccgtctctgggaggctaggattaatatagagctttgtttctcacctaatgggaatctactagcagcctgaaaaggcaggagccatgaaagccaatttggattttacatatttttcccctttatgttacagtacaggagggcaaaccctctcactggtgggattcctggcatcctagagcaggtggagagaagagttactttccactgtgggtagtggaggctccacctgtcccattaacttctacctcaatttgacttttattaagagcagggaaccacaatgacatgaaaatagacactataaacctcattttaattctttcacagaaagcttaggaattcagtgagttgtggcaacatggtttccattgtctaacatttttaaatgaattgatatggtttaaattcattcatttttaaaccagaattttttggagatagactatttccagcatgttccttctggatggtaaaacagggctgttagttcagtatttgtgacaataagtgtgtgtaaaataatgtcacctttcctgaatgtcaggaatatgagtctaatgcacaaatgtatacctctaagacaagactgcacgtcttttcaaatatacctgtccggccatttattttaataactccttttcgaatatacctgcttagcagattgtcttaaactctcaggacaggggagtaagcaagactgtgagccagtgacgatagcaaaggcttccaggtaggatccatatgaagtgagaaaatattcctcagctctcagggtagaactccaaagagatattcatgggtcctggccccaccgtggaggtcactcaaagggcaaacaggttggcatctcatctgcttcaagcctggacacaggggcaccatctgtgtcactctgtgtgtggtctgccatgttgtgggccggtcactacagactcgggcagccaggcagacaatgccttagccttagacaatgctggtgcagcccaggagtcagaaaatgcagtgtagaccaggccctccttaggccaacacaattacatgcaatagatgactggcttttctgttagtctcttcactggacccaaaggctgcattactctaccagaggggagctggaaagaaactaaagagttcgcccagcacagcatctgccttgacatggtaccatgtgaatctagacactcaccaagatctttccttgggggccaatgctgctgacacattaactcaatagcttgtcctcacctgagaggtcaggtaatgtgtttaaagttcaggagcagagattagtgtcattgatttgacatggctgtgacaacaaaggagggaactgaagtgggaatacccaaggccaccctggctttggcaggtggtgcacgcacttccactaactgttctggggcagggaaccaaatgtatgactgggcctgctcatgctgcccctgctgagtcctccaaaccctgcccttcatgtaatttctcagttttattttatcacattttataagtcactggatgtttacaaaatgtttggaacctatactgccttgaaggctaacctctaaagaggagtaaacaaggtcttaatacaactctccgggacgttttatcattacttatcttatatgccatactgcaccatttgctatcaacaggaaagtacctggactttggaaggtccctctgtgtcttttagctgaaagtacatatgaggcatgtggattcttttatgcacatcatctttttcagccacatttttgtagtttgcctctctggagccaactgtgtggggctagcagcttcacagctgaatcagtgtctggcaacctcttccttcagcctctcttcttcctccagttttccatccctcagtcacaccggagggggaaggtctgcaaggatccagaaccatcagttggaggagtttgcacatgactcatgaaagatgagttccaggcaggcctgccatagtgaacaccaggcttaatgggtttttcctcagagatacttcacgtacagaggcagtgaactgactgctttctggttgaccaccttgaaaaagatgagtgtgcctggcactgtgcttctcaggtgagtatgacctgagaagtattagttgctggttcttctgcacacaatcattcaaggacatatggatcaaccatcctcctcaacagctcaaatcaaccagatcatctgaccacagagactgaggtgtacctgaaagctgcccacatttctataaggccaatagaagccatgaacacagttgtcaatctgtagaaataaggactccatgactcctccaaggcctctctgtgaatgaacgtttaagaagggctagatcctaaaacagggtcagagcttagagggaagaaaaagcataaacatttctgagcaaattgtaagggcagtgtcaccataggctcccagtgaccctctgtgattgagtgcatacagtgatgcaaaatctcatcatcagtgcaaaagacaaaaaaaatcttactctttctacctaggatgagagtccccaaatcagcgaagagtccacttactaaacagacataaggaaatgaagtgtcctggaagaattcctgcctgaacctctcaggagcatttgaggacatttatcaagtattcactccaggattgggactatgaagacttcagctgctttcagctaatcattgagacttttcaggggtctcagaatagtcaggaaaggacctgatgagtgaatgcaattactgatgttggagttgctgttattatttatcgtgtacatattacctccctctcttgaccattccagttcctgagtaactcaccagccctctgatctataaagtcacaatccctgtgacctgatttctgtttcactttgtagatatgggacccgtacacatggactttttaagagactgggaattccagggcccacacctctgcctttgttgggaaatgttttgtcctatcgtcaggtgagttgcttgagcttcctcttttgcttcttatggttgcaaacatcagcttagttccatcagtaaaaatgcccctccttgggagggagttctgaggtttcacattttcagaaatggtgggactgggtgcagtggatcatgcctgtaatctcagcctctgtgaggccaagactggcaaattgcttgagcccaggagtttgagaacagcctgggcaacacagtgagacacctgtctctagaaagaaaaaattacctgtgcatgatatggtagcccatgcctgtagtcccagctactctgaatgttaaggtgggaggattgtatgaacccaggaagtcaaggctgtattgagctgtgatcgcaccactgcactccagcttggtcaacagaacaagacagaaaggaagaaagaaagagagagagagaaagaaagagagaggaaggagaggggaggggaggggaggggaggggggaggagaggagaggagagaaaaggagaggagagaggagaggagaggaaaaggtgtgtaggctccacccaaagcatggccaggtttacccctggagggaaagtcacaagctcatgtccagaaggccagtagcagcaagctgctctccagcccagatttcctatcctgtgtacctggagcttgtttctcagattctaactctcacaactgaagcctctgttgtctgattactatctgagaattctacacaattttaccctcgataaaagcagtaatttcttcttcatctttcccagatcaactcttgtagtagatcaacatttctgggaccttcttttgcatggttaaaacatcacagctgaatcttagcaacaggaaggtttgtttttatgtttcagaagtgaaagctcagagcacgcattgtaatttgctgggtgtgatgtgtagaggtggcatttctccatcttttctgtgttaagctagaaaactggaaaggaagtctactttctcattcactcactcactttctcactcaacaacatgccttagacttatctaaatctgcaagactaaaagaggttcctggtttctttaactttctaattctgctagagttctagagagagcacatgagataaatgaaaaggatactgatggaggagattaaaaaattgtgcattccctgcagacactcacttttcctcacctcagtttcacccctgcccttgcaggtgatcattcacggggttaggagactttagagagaataaaagaaaaagcaaaaatacatcagaaagacaaggaattacttactggtcatagacaagggtgagtccttcagtacttagagaaaattcaagagtgactttaaattccccacttcaaatatattctctgttttcttgtctttcccttaagacatctctgaatagcttccttcaactgccagtgaaagatagcaggcctgatttcattggacgcaactgttttcagccccaattagaggtagggtttattctatttaaaataataatcaacttgtattttgtttcctctcccagggtctctggaaatttgacacagagtgctataaaaagtatggaaaaatgtgggggtgagtattctgaaaacctccattggatagacctgctactgtgaggaggttaccccactgcaggatagtctctgcccaggtcttcatgggatgaagctcttgtcaacctaaatacaaacagagagaggttctctgaaagaagaggataattacttgggagtagaatattgcaatgggaatctgcttgccgttataaactatgtgcaaattcagggaggtaaacaagacaaagatgctccatagaaaatatgagaagaatctcataactgttttgagataattattgttagctacaaagatcaataacaagggtgatgccacaccaaggttggacaggcagttgctggacaggtgtccttgcagaaatatttttgtgtaaagttgaaatagcctttgtgcaaagttgtggtttttgtagacacttttgtaatagttttgtttccaggaacacaagcataagaatcctctcttcatagccttcttgggatttatttgtcagggttaaaaaacaattagtgacatcactttggttctgataaagttcacactcgctattgtaaaacttttcgaggcttgtcctaccaaggatcccatgtgtcaccaggtatcgaggtcttcagtctgaactaggctaggagcattgtggttaccacttttctgcaggttttggtggcccagggactcccagcatcgccttctgtccagtgtctgcctattcccctcttctttttttcttccttaggtgcccttttatcacatgcattgtctcagacccttctaatatgtgctcataaatgcatggcatcatctccttcccacattgattcactttcaattaaaagccaaaactccttcatttagactgaatttaacatgtgcttttgaaagaagggttgagagataatagagaaacagattgggaaaccacttatgctccacttttttaaactttctctgcaagtatggaattttttgttctgctttgttgtttaaatttaagccaaaacttcttaatagaaggatatacaaatatttattggtttataccattgcacttactttgaagaagagatgctgaatattattaaaccattgtgttccctggtgggctgatggactgtgattttataaggtggtctcagccaattgcagcagctgttccctgtcagaggggctagaggtttggtgagagcagtggatgaggtgcagtggtgtgtttgttcactagaagcaagtgggagaaagctttgcctctttgtacttcttcatcttctcccctcaagtcctcagaatccacagcgctgactgtggagtgctgtggagctggcatggcccatacaggcaacatgacttagtagacagatgacacagctctagatgtccatgggccccacaccaactgcccttgcagcatttagtccttgtgagcacttgatgatttacctgccttcaatttttcactgacctaatattctttttgataatgaagtattttaaacatataaaacattatggagagtggcataggagatacccacgtatgtaccacccagcttaacgaatgctctactgtcatttctaaccataatctctttaaagagctcttttgtctttcaatatctcttccctgtttggaccacattacccttcatcatatgaagccttgggtggctcctgtgtgagactcttgctgtgtgtcacaccctaatgaactagaacctaaggttgctgtgtgtcgtacaactaggggtatggattacataacataatgatcaaagtctggcttcctgggtgtggctccagctgcagaatcgggctagtgaagtttaatcagctccgttgtccccacacagaacgtatgaaggtcaactccctgtgctggccatcacagatcccgacgtgatcagaacagtgctagtgaaagaatgttattctgtcttcacaaatcgaagggtaagcatccattttttgaaatttaaataatgattgatccactgattaaatttttattttgaaaaaaacatatattcacagaaggttacctaaaaaatgtacaggaaggttccatgtactcttcatcctgtcccgcccagtggtaacatcttgcaatcttgtatattgcaatatatatctagtatattcatattatcaggttggcacaaaagttaaaatggcaaactacaggctgggcataatggctcatgcctgtaatcccagcactttgggaggccgaggcaggtggatcacgaggtcaggagttcgagatcagcctgaccaacatggtgaaaccccatctctactaaaaatacaaaaattagctgcgtgtggtggcatgcgcctgtagtcccagctactcagtagtctgagacaggagaatcgcttgaacctgggaggcggaggttgcagtgagccgagatcacgccattatactccagtctgggcaacccaatgagactccatctcaaacaacaacaacaacaacaacaacaaaaaccggcaaactgcaataacttttgcaccaacctaatactatagtacaggaaattgactttgatatagtttacagagcttttcagatttcaccagttttacatgcccttgtttgtgtgtgtttatgtgtgtgggtagttctaagcaatttttcacattcgtagatttgtgcaacgaccagcaccatcaagatgcagacccattccgtcaccatgtggctccctcctgctgtcctacagtcacaacatggagtttgtctttttctctgacaggttctatatcagagcaaacttttatttatttgaggaggccaatgtattaatatttccttttatggattgttcttttggtgttaagtctgaaaatcctttgcttagccctccttcctacattgctttttctaagagttatatagtttaacactttacaaaatgtaactctattacccattttgtgttaatatttgcataagttatgagatttagatcaaggttcattttctgtggactatggctgtccaaatgttccaacaccattttggaaaggtaggcatattgtcaaaactcagctgagtatattttgtgaatctatttcttattgtttactcctccactaataccacactgtggtgactctagtagctgtacagtaactcttaacatcatatagggcaattctttccactttattgatttatattttcagaatggctttagcttttcttgtcccttgcctttccataaaaattcagaataagcttgtaagtgtctacaaacaaacctgccataattttgataagaattaaagcagaggtgtccaatcttttggcttccctgggccacagtggaagaagaagtgtcgtgggccacacataaaatacacacacacacacacacacacacacacacacacacacacacacacaaatggtctgtgtatagttttcattatatatctaccaccacagataagcaaaaatgtccttgcataataatcctaattatgcactgccccattcagagggtctttcaaaatcattgaacaggttccaagtttgcaatcactgatacagaaaatgtacatatctagctaaacttcactacttttttgatattttttattataaaagaaaagagaacaacataaaactagtggggtacttgacattgtttttgagaaactaatccatcagtatctggcttgatggaagtagttgcaattctcagtgagttctcaaggtgctcatcagatattttggttctaattttactcttcgtgttcttcatccttgaaaatagtagctcacaaatgtaagtgctgccaaaaagcaatgacatgaacaaggtgtgattgtgaagcaagggatatttgtcattgggaagacaggtcttacaaaagtccagtaaagaggcaaaatcaaatttttctataagttgaacatcagattgcagctctaggcattccatttcaaaattgccaggtaacatatatatgtcgactgaaaatggagttgcaaatataccaaaatattgatgattttttcagaaatcttgaaatacctgttttcaaattcctgtatcaaattgaaaagcaaggctgcgtatttttggctgttcacaggaccatgtttagccaacatgtcgaaatgcataaaattgtttgccttaatttgagcttgccataatttcagtttcatatggaatgctgttatggtttgaaacattgtattgttaagttggttttcaacttgaagacacaggtttaactcacttaaatgggccgtcaaacccactaaaaatgctaaatctgtaagccagttttcattgtcaagttctggcaccaattttgtttgataccataaacagcttgatttcacatcacaaagcataaaatctttacattttgccttgacttaaccatcttacttctaaaaagtgaatgacttgctagagtcagcatccatacttttaaggaattcctgaaactagcgatgattcaattcctgggcccttgtgaaatttacagccttgatgacaatttgcatgacgttatctacttttaaagcttgtgcacatggattttcttgatgtattatgcaataatacttcatcaaatgtgagttttgtgtggcaactgcatcatctattaattgtacaagtccctctcttttacctaccatcgccagggcagcatctgtagctatatcacatatgtttacaaaggacaaagaaaattgctttaacatatttttcactgcttcatataaatctcttgatttagttgtgtcttttaatagcatggtgacatttcgatttcttcagtgacattatattcatcatcaatacctctaataaaaatagcaagttgtgccgtatctgtagtgtcagtgccttcatccatcaccaaagcataaaattttaaattagcagttttactctccaaacgtctttcaatagatttcccaatttctccaattctcctggctatagtctggtgagacaaactgattttagaaatatcagtttctcaaggcaaataatatctaccacatcttccagacattgcttaataaactcaccatcagtaaatggttttgatttttttgctattaaatttgctaccacataactaggttttaccttacgattgagtccgagttgtaactttttaaaaaatcttttttgttgaaaagacagactttttttcagttctgctattttgtccttacaacacatacacaccaaatttgtcagcacgtttttgcatataatgcctcttcaaattgtagtctttgaaaactggcacaaattccgtggaaattaagcagagtgcttcgctatttgcctcaacaagaaaaagtcatttgtccacttttcattgaacaatcttccttcatccataatttttgttttttagggttttctttttaagacattgtggaagccattctggaattaaaagcattataatagataagcaactatatttacttttattatggaaattaacagataggaaaatagaacagaaagcaaggtttaataatcaaataagaatacttacatgtcttctaaataatattaaacacctatcatctacaaaggtaggttgaaatattattgataattgctgggttttacttgccaaattgccacaaacacacctaatacctgacagtgtcaattcaactgtccgtgattagaagataacacactggaagtcgcacaccaccataaaactgaagccacacatgcgtacaaatggcgacagtgtctggtgtacagcagcgctctgccttgtccagaatacacacttgaattctttgtcacaattcacttcacgtggcactgcaatagcgtcctctcgctctttgttagttaattttaatggcttttaatttcttcttgctgaactgtttgcaattataatgcaaattatggatactagtccattatttgtggatgtgacatactctgattacccctttccattccattgttgtctacgaagttcacacttgagaatcacatagtcaaattacaaaattacaaaaaaaattgcaaaaaaactcaaaatgttttaagaaagtttccacatttgtattgggatacattcaaagccatcctggactgcatgaggcctgcaggccacaagttggacaagcttgaattaaaccaatagaacaatttgggtataatctatatctttactatgttcagcctttcatcccgtgaatatagtatgcctctccatttctttagcttttattactttcctcaacattttatagttttcagcatagaggtcctgtacatcttttgttagatttacaccagaaatatttcatttttgttggagtaactgtaaatgatactgtttttcttgtattttcagatattgattattgttacatagaaatgtgaataattttgtttgttgatcttgtatcctatagccttgcagaacttacctattcgttctagaaatttttttgtatattccttgacattttatacattgacaattatgtcacctgaaaatagagacaattctattatttcctttccaatctgtatgccttttatttctttttcttgtctagtgtattaagacatcaggtatgctctttagtaagaatgttgagagtgggcattttttagttcttcttgatcttggaaaaaccattcagtccttcatcattaaatgtgatttaactgaatgatttttttacagattgtctttatcaaatgaaggaactgtctctctcttcctagtttattgagattttatcatgacagctggaagtacacattttaaaacaaaacatagttgtggaagataagagaaagttccaagcatgctggcttgatagtccagccccaagttgggaaaagtaattatccctttctttttccttctatttatggaataaaaaattaagagaaaagaattttcaaggaaattgcattattccttcaaaacaggtttctagtctttaagtattacctacttttcaaaaaaaaatcaccacatcatggcatccctttttcaagttgcccatgctgtaggtgtattaaagacagagctggtctgaggcaacatacagtctgcccatctgtcaccaatccttttctactctgcacactcctggggaagggctaggtcttgttcctgtctattccactggaagaacagttccctaccacgtggagcatttgcaattaaaaggagactgagatatagaggcaggagaccacaccagatggctgggtctccccactcccacccccgccccacatacactcagaagaggctaggcatctaggatctccattgagcatcttgaatatggcttgccataatatcatatacagtcaataaatatttgttaaataaggatgcctcttcaatatattttgtgcaaccatgaagatcaccacaactaatgtgagaaaaaatgtttctgttgaactctagtctttaggcccagtgggatttatgaaaagtgccatctctttagctgaggatgaagaatggaagagaatacggtcattgctgtctccaaccttcaccagcggaaaactcaaggaggtatgaaaataagatgagtcttaattagaaatgtaaagaatgaatctggggacaggtagaaagtaagatcacagtccgtttccaaggggtagtccactgagttcgagcttcctaaaaatggtcttttatctttatgtacagaaaagacatcacaaaattcattacaaaatgtcacttactgctccatgctggagaaagccatatccttctgggacttgagtctgcacatttaactacaggtactgatctgttttgtgcttagatgttccccatcattgcccagtatggagatgtattggtgagaaacttgaggcgggaagcagagaaaggcaagcctgtcaccttgaaagagtaagtaggagcacagccatggggttctgagctgtcatgagcccttccagctgcctgccatggagtcgacagtcgcactgttgggttactccagtgaccagacaaaagcagggcagcgctgcaactccaaagagccacctaagagggagtggctcccatgaggcggcaagtcagcaagggaaaagggccttctctcctgtgcacaggagccaggatttacttatctgttaacttgtcaccataaatattctgggagattaaatacatactttagaaattaaaaaaacatgattgtatcaaagttttgagtgtagtggatatggaactgtgggtaagcaagcatttggtacttgttgccttgcattgggtaagatgggaaagttacaatggggaacttggaacaatttcaatcccttcatggtttttctgagaatatcagcaaactatgaactattaaaccttcccactacttccttttcctccaatctcaaaaaagaaagggtgctagaaatgctatgtgtagagcaagcctattatttgctgtctacaatggtatgtgcttcaattatgcaggaacgacaggtgtaatctgagcctgtcctgttcagacttgggacatgtggtcactcagttttgggttctccaaatcaatgttggagagatctattttttttaaccagaacattcttgattgtcacatcttacaaaaatgactctgctctcagcgcaacttcaggtcagaggagctggggatagtggggttttccagagcattagcagggagtgtagagaataaaggatgatatttctaggaactcagaacagggtgttactgttttgtaaagtgttgaagaggaattggctctgggcatagagtctgtagtcagacaacgccacctttcttgaatccactaggaagagttaattattctactcttgttctgctgaagcacagagcttacatatcttatatcatccacactcaacacatgctactgtagttgtctgataatgggtctctgtcttcctatgactgggctccttgacctcagaggtgagtctaactcagcttggtgtctccatcacccccagcatagggccagctccatcactggcaccagataaccaccttctgagggagtagatggaagatgattcagcagatagttctgaaagtctgtggctctttatgtgtcttgactggatatgtgggtttcttgctgcatgtatagtggaaggacggtaagaggtgctgattttaattttccatatctttctccactcagcatctttggggcctacagcatggatgtgattactggcacatcatttggagtgaacatcgactctctcaacaatccacaagacccctttgtggagagcactaagaagttcctaaaatttggtttcttagatccattatttctctcaataagtatgtgggctattatttctttctctctttttaaaaataactgctttcttgacatataattcacatatcgtataattcatccacttaaaaggtacaattccattgtttttaagataatcaaaaatatgtatgaccattactattgtaaactaaaatgtttttgtcaatctagagccctcacacactttagctgtcaacaccccaccacaaaccccactgccctaagcatccaataatcaactttctgcctctatagatttgcctattctggacacttcatagaaataatatcattgatttttctctgttgttttttattctctatttcatgagtttattttagtctgttattttctttcttttgctggctttaggtttcatttgctcttcttcttttagtgttttgtggtgtaaataattataatcaatttgagatattttcttcttttaaatttagatattacagctataaatttccctctgagcactggtttggctacatcctgtgttttggtacatcatgccttctttttgttcatctcaaaacaatttcttgttgcccttttgatttctgctttgactcactggtcacttaaaactgtattgtttaacttccacaaatgtatgagtttcccaaatttctttcccttattgatttctagttttattccatggaagttgatgtacatatgctgtgttaattctatcttgactatcatttcctgaacagcatgattaagttaagcagcagattatggtctacattaatccaaaaactctagtccaatagataaaggctaagaggtcagggaatttaattctattactttggtcactccaaagactcagaaggtgccattgatctcactgctgtagtggtgtttcctatgtatagacctgcccttgctcagtcgccggcctgaaagaagggcaaacatgataaaaggaatgggttccagttgagaatcatgatgttcttattcttattactggtagagaaaattataattgctccaggtaaagtttgcattttcaatgatttccttttgtttgttttgtttttcccacagtactctttccattccttaccccagtttttgaagcattaaatgtctctctgtttccaaaagataccataaattttttaagtaaatctgtaaacagaatgaagaaaagtcgcctcaacgacaaacaaaaggtaaaatctgatggtggttaaatgacgatgtttaggttttgataaatttagattttatacacatgatagagcatgtatctgtatttttaaaaataaagacagagaacttatgtttagaacaagagaagccatttggtagaaataaagaaggagattggggaaggagatgagaatgagtcagagagatagcatttaaaacttgaaatcaggcacaacaattagtatgtcatgatataaacagtattgagataaaattttaccacttctcttccctttaataaattgtcaaaggataaagtttcctgtttgaaaatatattttactggtattgtgctttcctcatatcacagattggtaaagaatcattttaagtccaagactcttattttacatattctgcaattaaaggtcctatgaggctacctgccgactgctgacatgtagtgtgtggtaaatgtgagtgtttcacagcctggagtgaacaggggtcttctctgagaattgaggttgcaaggctggctaactcagctttgccttcacgagccctagaggccagccgaaggatgtctgcaggtcagggagacaggaccaggtaacccagctgtcactgaagattatatagagtttgagaatgttggaatatttgaaaatgctcccccaaaaaagctgctgatgagttctggaaatgtcaggagattaatctatacggacactgctgaagaaaaaggtagaagaataaaagatccagtacttcttcctgggtaagcagttatgaccagagatggaaccggcaactctttggccagaaagctgtatccaaaagacagagaagatgagaaacagggagggcaaaggcgaaaaagcaattggacatgatagctagatttgtttcaggaaaacatcctgctttccaaggatttagatgaatgtttttgttcactggtgactcaggtaacacgtcttcaagaagccatagggaggttgagggagggaagtcaagaagggaggttgaggactgcacttttgatttacttctgacttcacgagtcactttctgccaaagaaatctctccttttgcttctagcaccgactagatttccttcagctgatgattgactcccagaattcgaaagaaactgagtcccacaaaggtaaccaaggagtgcttctgagggctactggcggggacactaagagggagggccttgttctgaaaatgtgcaggaagtattccaggaagatgagaatttttgccacatagcagaacaacacacatttagatgttataaatggtagctggaggcactttccagaagcccacaggtatagccatgttccaggctgaaagggcaaccctaagcaaacctagaatgcttggaggacagtcagtggtttgtggatcacctacatgagatcaaatgccagttctcagcctcctccagatccaccaagtgagaacctctacttggaaatttatatcaaacataccgatcaggaagcacactatcccagtaagggtgattttaactggcagtacttgaaagtgtgttcgcaaggttaatctactgcaaagttttatttttccctttgaaatgcataagtaactaatgggggacacctctgataccatgtaaatctacttcaatcttcagtcttgtatctactagttttatgacccatggatggttttaaccaaaaccattattactaagacagtggcaaaatgataaccatggtcaatttcaagctaccaagatttggcaaccatctcacaaaatttttgaatatttaacaattggttctagagagcaggactcagcagactccagtataccactttaaacatgtccatgtctacatctacttctgtctgtctatctatctgtcaatcatctatctgcctataatttatcaattaatcatctatctatctcaacaaaacttgctgtgataaagaaaatagtctatcatttcactgtttcatatagaaatcactagacacatatggctattgagtactggacatgtggccaatgccactgaagaacaatttttaagagtatttatttttaattgaataaaatttgaatttaaatagccacatgtggatagtggctaccagattggacagcagagctcccaactttaaaattacagttcaatttcaactcagtataatggggttcaatgtaactgagtaaaataattggatggttgaatttacccacagcagcatacagaaatattcactgataaatcagaactctgtagacctttctcacactcattttatattgtgtttggttgtgagttacatgattgctgcaggcaccatatttatttctgtgctccaggtctctaaaggtcctaatccagtcctgaccaaacagactagtgatggaccatcgtgagcttctctcaggagaaatatcaagagggaggccaacctgtaatcataagaacttctgctattttaatgccattcatcagactacagtcaatcaccatgcttctggctttttgtctatctctgctgtcttgtacatcctgagatagtccattctgagaactgtaccctagatcttgtattgcctgatgcctgtcaaagatgtaatccatgctgcttaagtgaggttgtgcacacaaatcaccatatctcctgcaagtttggattttgattcagtagttcgatggtggggtttgagattctgcatttctaataagctcccagatgtggctggtgctgctggtccatgaaacacactttgagtagcaagaggtgatctgtagctcagtattggtcctttaagttccctcaaacatatatagagaaaaggtcctaaatattgcaaattctctcaaagtttgtcaagctatattggaattctctcaaagtctgtcaagctctattgtagaaaatcaaatttttattgggaaaaagcctaccccatatttacttacagataaagtacttttaggatcattcaaggcacacacccataacactgagtatgtaagacagaaatgctctctctggaaattacagcagtgctggtgctgggatgccatgatgaggagtgtgtggcccacaatcatgtagaccttgggaaaacctggattaaaatgattttgcgtcatcctggccctgtataagatacatatcagaatgaaaaccactcccagtgtgactttgaattgcttttccattttttcttcttgggattagagagcttcacttagatttcatctaagctgtgatgttgtacgttgacctgatttacctaaaatgtctttcctctcctttcagctctgtctgatctggagctcgcagcccagtcaataatcttcatttttgctggctatgaaaccaccagcagtgttctttccttcactttatatgaactggccactcaccctgatgtccagcagaaactgcaaaaggagattgatgcagttttgcccaataaggtgaggggatgacccctggagatgaagggaagaggtgaagccttagcaaaaatgcctcctcaccactccccaggagaatttttataaaaagcataatcactgattccttcactgacataatgtaggaagcctctgaggagaaaaacaaagggagaaacatagagaacggttgctactggcagaagcataagatctttgtacaatattgctggccctggttcacctgtttactgttatcacaataatgctaagtaaaaaaaaaaaaaaaaaaaaaaaaaaaaaaaggagtgtggcgagaagatggccaaacaggaacagctccagtctacagctcccagcgtgagcaacacagaagacgaatgatttctgcatttccaactgaggtaccgggtgcatctcaatggggattgttggagagtgggtgcaggacagtgggtgcagtgcacccagcctgagccaaagcagggcgaggcatcacctcacctgggaagtgcaaggggtcagggaattccctttcctaggggtgacggacagcacctggaaaatcaggtcactcccaccctaatactgcgcttttctgatggtcttagcaaacggcacaccaggagattatatcccgcgcatggctcggagggtcctacgcccatggagcctcgctcattgctagcacagcagtctgagatcgaactgcaaggcagcagcaaggctgggggaggggcgcccgccattgctaaggcttgagtaggtaaacaaagctgccaggaagctcaaactgggtgaagcccaccgcagctcaaggaggtctgcctgcctctgtagactccacctctaggggcagagcatagccaaccaaaaggcagcagaaacctctgcagacttaaatgtccctgtctgacagctttgaagagagtagtggttctcccagcacacagctggagatctgagaacagacagactgcctcctcaagtgggtccctgacccccgagcagcctaactgggaggcaccccccagtaggggcagactgacacctcacacggccgggtactcctctgagacaaaacttccagaggaatgatcaggcagcagcatttgcgggtcaccaataccgctgttctgcagcctccactcctgatacccaggcaaacagggtctggagtggacctccggcaaactccaacagacctgcagctgaggatcctgactgtcagaaggaaaactaacaaacagaaaggacatccacaccaaaacccatctgtacatcaccatcatcaaagatcaaaggtagataaaaacacaaagatgggggaaaaacagcagaaaaactgaaaaatctaaaaatcagagcacctctcctcctccaaaggaacgcagctccgcaccagcaacggaaagctggatggagaatgactttgacgagttgagagaagaaggcttcagacgatcaaactactccgagctaaaggaggaagttcgaacccatggcaaagaagttaaaaaccttgaaaaaagattagacaaatggctaactagaataatcaatgcagagaagtccttaaaggacctgatggagctgaagaccatggcacgagaactacgtgatgaatgcacaagcctcagtagccaattcaatcaactggaagaaagggtatcagtgatggaagatcaaatgaatgaaatgaagaaagaagagaagtttagaagaaaaagaataaaaagaaaggaacaaagcctccaagaaatatgggactatgtgaaaagaccaaatctacgtctgattggtgtacctgaaagtgacggggagaatagaacgaagttggaaaacactctgcaggatattatccaggagaacttccccaatctagcaaggcaggccaacattcaaattcaggaaatacagagaacgccacaaagatactcctcgagaagagcaactccaagacacataattgtcagattcaccaaagttgaaatgaaggaaaaaatgttaagggcagccagagagaaaggtcgggttacccacaaacacaaacccatcagactaacagtggatctctcggcagaaactctacaagccagtagagagtgggggccaatattcaacattcttaaagaaaagaattttcaacccagaatttcatttccagccaaactaagcttcataagtgaaggagaaataaaatactttacagacaagcaaatgctgagagattttgtcaccaccaggcctgccctaaaagagctcttgaaggaagcactaaacatggaaaggaacaactggtaccagccactgcaaaaacatgccaaattgtaaagaccatcgaggctaaggagaaactgcatcaactaacgagcaaaataatcagctaacatcataatgacaggatcaaattcacatataaaaatattaaccttaaatgtaaacgggctaaatgctccaattaaaagacacagactggcaaactggatagagtcaagacccatcggtgtgctgtattcaggaaacccatctcacgtgcaaagtaacacataggctcaaaataaagggatggaggaagatctaccaagcaaatggacaacaaaaaaaggcaggggttgcaatcctactctctgataaaacaggctttaaaccaacaaagatcaaaagagacaaagaaggccattacataatggtaaagggatcaattcaacaagaagagctaactatcctaaatatatatgcacccaatacaggagcacccagattcatgaagcaagtctttagagacttacaaagagagttagactcccacacaataataatggaagactttaacaccacactgtcaacactagacagatcaacaggacagaaagttaagaaggatatccaggaattgaactcagctctgcacaaagtggacataatagacatctacagaactctccaccccaaatcaacagaatatacattcttttcagcaccacaccacacctattccaaaattaaccacatagttggaagtaaagcactcctcagcaaatgtaaaagaacagacattataacaaactgtctctcagaccacagtgcaatcaaactagaactcaggattcagaaactcactcaaaaccgctcaactacatggaaactgaacaacctgctcctgaatgactactgggtacataacgaaatgaaggcagaaataaagatgttctttgaaaccaacaagaacaaagacacaacataccagaatctctgggccacattcaaagcaatgtgtagagggaaatttatagcactaaatgcctacaagagaaagcaggaaagatctaacattgacaccctaacatcacaatgaaaagaactagagaagcaggagcaaacacattcaaaagatagcagaaggcaagaaataactaagatcagagcagaactgaaggaaacagagacacaaaaaaacccttcaaaaaaatcaatgaatccaggagctggttttttgaaaagatcaacaaaattgatagaatgctagcaagactaataaagaagaaaagagagaagaatcaaatagatgcaataaaaatgataaaggggatatcaccacccatcccacagaaatacaaactaccatcagagaatactataaacacctctatgcaaataaactagaaaatctagaagaaatggataaattcctcgacacatacactctcccaagactaaaccaggaagaagttgaaactctgaatagaccaataacaggttctgaaattgaggcaataattaatagcttaccaaccaaaaaaagtccaggaccagatggattcaccgccgaattctaccagaggtacaaggaggacctggtaccattctttctgaaactattccaatcaatagaaaaagagggaatcctccctaactcattttatgaggccagcatcatcctgataccaaagcctggcagagacacaaccaaaaaagagaattttagaccaatatccctgatgaacagtgatacaaaaatcctcaataaaatactggcaaaccgaatccagcagcacatcaaaaagcttatccaccatgatcaagtgggcttcatccctgggatgcaaggctggttcaacatacgcaaatcaataaacataatccagcatataaacagaaccaacgacaaaacccacatgattatctcaatagatgcagaaaaggcctttaacaaaattcaacagcccttcatgctaaaaactctgaataaattaggtattgatggaacctatctcaaaataataagagcaaatttatgacaaacccacagccaatatcatactgaatggacaaaaactggaatcattccctttgaaaactggcacaagacagggatgccctctctcaccactcctattcaacatagtgttggaagttctggccagggcaatcaggcaagagaaagaaataaagggtattcaattaggaaaagaggaagtcaaattgtccctgtttgcagatgacatgattgtatatctagaaaaccccatcgtctcagcccaaaatctccttaagctgataaacaacttcagcaaagtatcaggatacaaaatcaatgtgcaaaaatcacaaatattcttatacaccaataacagacaaacagagagccaaatcatgagtgaactcccattcacaattgcttcaaagacaataaaatacctaggaattcaacttacaagggatgtgaaggacctcttcaaggagaattacaaaccactgctcaatgaaataaaagaagatacaaacaaatggaacaacattccatgctcatgggtaggaagaatcaatatcatgaaaatggccatactgcccaaggtaatttatagattcagtgccatcgccatcaagctaccaatgactttcttcacagaactggaaaaaactactttaaagttcatatggaaccaaaaaagagcccgcattgccaagtcaatcctaagccaaaagaacaaagccggaggcatcatgctacctgacttcaaactatactacaaggctacagtaaccaaaacagcatggtactggtaccaaaacagagatattgatcaatggagcagaacagagccctgagaaagaatgccacatatctacaaccatctgatctttgacaaacctgacaaaaacaagcagtggggaaaggattccctatttaataaatggtgctgggaaaactggctagccatatatagaaagctgaaactggatcccttccttacaccttatacaaaaattaattcaagatggattaaagacttacatgttagacctaaaaccataaaaaccctagaagaaaacctaggcaatatcattcaatacagaggcatgggcaaggacttcatgtctaaaacaccaaaagcaatggcaacaaaagccaaaattgacaaatgggatctaatgaaactaaagagcttctgcacagcaaaagaaactaccatcagagtgaacaggcaaccgacagaatgggagaaaatttttgcaacctactcatctgacaaagggctaatatccagaatctacaatgatctcaaacaaatttacaagaaaaaaacacaaccccatcaacaagtgggggaaggatatgaacagacacttctcaaaagacatttatgcagccaatagacacatgaaaaaatgttcatcatcactggccatcaaagaaatgcaaatcaaaaccacaatgagataccatctcacgccagttagaatggcgatcattaaaaagtcaggaaacaacaggtgctggagaggatgtggagaaaacaggaacacttttacactgttggtgggactgtaaactagttcaaccattgtggaagtcagtgtggtgattcctcagggatctagaactagaaataccatttgacccagccatcccattactgggtatatacccaaaggattataaatcatcctgctataaacacacatgcacacttatgtttattgcagcactattcacaatagcaaagacttggaaccaacccaaatgtccaataatgatagactggattaagaaaatgtggcacatatacaccatggaatgctatgcagccataaaaaatgatgagttcatgtcctttgtagagacatggatgaagctggaaaccatcattctcagcaaactatggcaaggacaaaaaaccaaacactgtatgttctcactcgtaggtgggaattgaacaatgagaacacatggacacaggaaggggaatatcacacactggggcctgttttggggtgggaggagtggggagggatagcattaggagatataccgaatgttaaatgacgagttaatgggtgcagcacaccaacatggcataggtatacatatgtaacaaacctgcacgttgtgtacatgtaccctaaaacttaaagtataaaaaaaaaaattcaaaaacctcagtggcatctaatgagaagcatttattgctcacaagactggatagtgagttctgctgatactgactggactcactctggtctggctatggtctgaggtagcctggccctgggggcgcgatggaggctgactcagctctccccacacctgtctcatgttccagtcaggtagccactggccaagaagccaagctaggaaccagggtatctgactcctgagctaaactctaaccctctacaatactgcctcccaaatataacaccaagtgctaggtacatatcatccacagttttcagacttctgcccaaactgggattctttttagtgtgaagagacctggcctgtggggctgaccctggtgtggctgtgaggcagacacaaagggacatttacatccagtcctgaagattacagtccagccctgaagcaacaactaggaaactattccaaaaggaggggatggggctgagtgtggggttctattctcttcataactttaactagaactcaaattgtgtaccttggtagcatccaatcataaatttattttgtcgtatttgtgatagaaaggaacaagtttatccacaaatttatttatttatttatttatttatttatttatttgagacagggtctgactctacgacccaagctggagggcagtggtgcaatctcagctcactgcaaactctgcctcccaggctcaagccatcctcccgcctctgtctcctgagtagctggaactacaggcacacgccaccacacccagctagtttttgtattttttgtagagatgggttttcaccatgtttcccaagctggtctcaaactcctcaaaagagttaccaagcaggactctgcaaccaataatccttgtgtgaagaggatatttgctcttttccctgtttttctttcttggtacagatgtgtgacctctttttgaaaggtgatagtgactttggtgtattttatttggtggtaatggtcatagccccattaatcacatttcttcccatgagaaagaaaaaccactacatggtcatgctaaggatttcagtccctggggtgaggatggtcttgaatatctcctacattcataactcctccacacatctcagtaggtcactgagcacatcaatggacatgccagttattaaaatacttcacgaatactatgatcatttaccagtatgagttattctctggagcttctaatacttcaatagtactgcatggactcagttgagagttaattcaaaatctcagattatccaattctgtttctttccttccaggcaccacctacctatgatgccgtggtacagatggagtaccttgacatggtggtgaatgaaacactcagattattcccagttgctattagacttgagaggacttgcaagaaagatgttgaaatcaatggggtattcattcccaaagggtcaatggtggtgattccaacttatgctcttcaccatgacccaaagtactggacagagcctgaggagttccgccctgaaaggtacaagtctccagggaaatggagctcaccctgacccaggctggttcaagcatattctgcctctcttaatctacatgacaatcgtgtggttgtacaatcatttgcttgtaagtctttttatcacaaaaaagtgataattatcaaactttacaaaccacagactagaaaaaacgaaactacatccatccacagtcccagcacaagacaaagataatcaattatgtccctgtgggcatttttctacgcctatatagatttttaaaaattagaatggtatcactttttatttggtttgaattgctgcttacttgatttaacaggaaactatccactgacctatattactataaatatacatatatatgtatatatataaatatatatatatgtatatattgcatatgccataaaccatttaaccatgatgttatttcaggtgtataggctttttattcctttctgttttttctatgctgtgccctttagctctctgaatttaacagaaactttaaaacatgcttccacattccatttgctttcaacgttacttgctatttcctctgtagtaattataagagtgcaggctgaggtcctgagaagtcctcatccctaatggtttaagccacttcactgaagacacaagacagcacaggtcctcctggtcctatctgtggctgcagtcctgtgccagctcccttatactctcagtagacatctcacacactcctccttggaggtgtcttgagcatgctcttctgggaattcagggacaaggtcaggccttaggcacagttcgcactctggatatagttggtgttttcccattactgtattattaagcaaaatttagaatgaaatttttagggtactggctggtgattcaggatgcttgggatctagactttcattagcccctacctgcaagtttgctgatgggaggaaccttgtcttgttggtcatggtgtccctagtgctagcatggagtctgcacataatacttgttcacagagtaagtcagagctgaccaagttctctgttttctggagtagaggacttctatgtttcctgcaagctcagcacttccacctcctgtggctgcactaatacgaaatcagagaccactcgctgtacttcactttgaatcactcagtcaccaaaaagatagtgcttgccatgtgtcaggaacttggctaggcagggagaaattcatatgatttatataaatccataaatccatatgatttacataaatccataaattcatgtgatatatacgtatatgtgtgtgtatatatatattagagaatgtttgacatatacacaagtacatgttaccgacaccagcctatagaatagttttcgtgcatctccatatatctatcactggttccaacagccatcaatccatgttagctgccccatccaaatgccaccatcaccctcctcctgactatcatgttattttgaagcaatagcctgtaaatatttcagaatgctctccaaaatataaagactcctgtaaaaacatatgacaacaatgccattattactttctttgaatcaacattttttccttaatataatcaaatatttagaaatcaaatttgaataaaacatgggtcaatcttcaaagaatttatagcttaatggaacagatcaaggaaagcagggatgacactacagtagggtagcatcatatgcccatgtaacttatgtgacttaaactatcctgtaagggtgtgggggagaaagagaggaagagatggagagaagaaaaaggaagagaaggaggaggagaaggaggcagaggagaaggtggacggggaaggtagagaggaggaggaggggaattagaaaaaaagagatgacaggagaaggaaagggaaaaataacaacttgaaatagcacaagacgttttctccttctcctttctcaatgagcatgtgaccaacacaagtgtgagttgaggcaggaatccacttttccatccatcagtcttatcatttatgtgccttttatagtgtgaacacatcaccaccctgaatataattttagtgtttagagataaatattatttgcaacaatattcatctcatctcaagaaacgctcctatagggtatggagaatttaaaggacctgtaggttatgatgattataacgaaataaccaaagcaggatttcaatgaccagcccacaaaagtatcctgtgtactactggttgggaggtggaggggggttgttcttaagtaagaacccctaacatgtaactctgtggtttttatgtttcattaactatttaatctaccaatatggaactaggttcagtaagaagaaggacagcatagatccttacatatacacaccctttggaactggacccagaaactgcattggcatgaggtttgctctcatgaacatgaaacttgctctaatcagagtccttcagaacttctccttcaaaccttgtaaagaaacacaggtcagtacactttctgtatgttttattaagaatttttttaactgaagggtatatattttttaaaagaatatgcatgtttatcttttaataattcattctatgggccaaagaacctacttggatccatctttgatcattaaggatgcttcagttctggacttcaaaacctgtagcattaagaacatcatgtaaagtccacacagattagcatgacatgattatgtgtagtctctttgaacctgagtaagtttaaattcagtttcaagtcaattggaaagaagtgttttgcacaatcatgaagtgcaatgattacctggctgtgacttaaatggtgttctccatcaccagaacctgcagaagctctctcatgacagtggttctcaaccactagctgtatattggaatcaccagggagcttcaaaaattcatgatgcctgtgacatctcagaaattctaaactaattaacccagagcgtgactaggttctgtcatgctgtcgggtgaacccctgattagttctcacgtgaagccaaggtggagaatgactaatttcaggcatttctggtggatatgaaggactaccatagagcagggctatccttactccttgaccttatgttccaggtgatacatttaaagaaagatttagaatcttttctctgaagaagttaaagaacagatgtcattgattcatattaagcaatagcctataagtcttatttccaggaccggtgtatttaatatgcaactctaccccttaagtacactttgtgcttgggagaggaggaggatggagatggttgccatcttatctatggcttcagggcagctgtgtagctttcctatgtgtgtattcaggcagggggctcagccctgagagaaagtgggcctctggcacacctgggacagggaagatattccctggcaagctctcaggcatctcaggctggcacttctttgtatccatggcaatttgctttcccctcactgaactgagatcagaatgttactctgttggtggctcccccaacagtgaaggggtgactcagtgacaatagtgctagaagtatgagtcaaaacactgtacaacttgagaaattccccgtttgcactacgcttggaagccaagaggagatgttaaaaagaaaagaataattctttctgaagacatttcccatcattgcacttgatgggttcaactgggaagggttactagactctggaagttgaaaactgcccacataattaaactgtacaacagctactcaggattaccttgcaagttttaacctataaaaatttaactttatatagcacttccaaaatagtttgccataatacctactaatctggatttaatttttaaaactcatccttttaacttaagatttaaataaaaaaaaaaaaacacgagtccacaagaatttgtctcaggcctggcacagagtcagtgctccataaatattttgttaaacgatggatggtgagtgcttttactatccagtatttacccagcttatagattaagtatgaagagttcaagatacatggtgttaagagtcgtttttatatgcttgcaaagcatttttgtcatattttttctactttgcttccatcttttcttctttcacttcatttattaattctccatatgcttgtttaactattgtagatccccttgaaattagacacgcaaggacttcttcaaccagaaaaacccattgttctaaaggtggattcaagagatggaaccctaagtggagaatgagttattctaaggatttctactttggtcttcaagaaagctgtgccccagaacaccagagatttcaacttagtcaataaaaccttgaaataaagatgggcttaatctaatgta

SEQ ID NO: 3 - DNA, 24 bp, Homo sapiens
acccagcttaacgaatgctctact

SEQ ID NO: 4 - DNA, 23 bp, Homo sapiens
gaagggtaatgtggtccaaacag

SEQ ID NO: 5 - DNA, 19 bp, Homo sapiens
tgtctttcaatatctcttc

SEQ ID NO: 6 - DNA, 18 bp, Homo sapiens
tgtctttcagtatctctt

SEQ ID NO: 7 - DNA, 24 bp, Bacteriophage M13
agcggataacaatttcacacagga

SEQ ID NO: 8 - DNA, 39 bp, Homo sapiens
gagctcttttgtctttcaatatctcttccctgtttggac

SEQ ID NO: 9 - DNA, 24 bp, Bacteriophage M13
cgccagggttttcccagtcacgac

SEQ ID NO: 10 - DNA, 39 bp, Homo sapiens
gtccaaacagggaagagatattgaaagacaaaagagctc

SEQ ID NO: 11 - DNA, 7218 bp, Homo sapiens
tggaaaatgcagcgtttttatttctttttctaaatatgtaactcttcctccacttccccctctcctgcttgccttatttcaattgcaagcagaagagagtgagtgttctctgccggcaaactccgccagggtcccggcccgtagagagtcgtcaagggtctggaacccccgtgccaacacctgcccctgcttcgcagccccaagaggaaggccgcgtctttccccctcgctgtattgggaagctacgttccgggctggccaaatgggccccaattttccaaaacccaaatttgtaatacccttcaattttttaaaaaaaagaatttaaaaaagtctctgtgaatgcttcagaagttaccgtttacaccccagaagtacttgcagcacatccacaagtaaaaacacacaacgaatgccagagtttcgtgtgttttttaaccgacatctttgtggctgtgaacaaacttcataaataaaatagaatcaaatgcttctgacctagagagctgggtctgcaaactttttttttatcgtattccgcaacagttaaataaaaaattaaaaactcaacatgtctccttgtaaactacatcaattaacaaacacactatgtccattatcaaatataatagaaaaaatataggaaaatagaaaatagaaaaatataggaaaatagaaacttttaagccacggtgaaaatgtttctataaatgagtggttctaatgttttcgtgagcgcccattttggggagcaccgccagctgcccgttcaggagtgtgcagcaaactcagctgagagagaaaattggaacaaaagcaggtgctcgcgggtacctgggcctagcctcttagtgcggccagccaggccaatcacggcccccggctgaaccacgtggggccccgcggagcctatggtgcggcggccggcccgccggtccgcgctggctgtgggttccctctgagatcagtgcggagctgtcaaagcgagcaggggtggcgccgggagtgggaacgccacacagtgccaaatccccggctccagctcccgactcccggctcccggctcccggctcccggtgcccaatcccgggccgcagccatgaacggcgaggagcagtactacgcggccacgcagctttacaaggacccatgcgcgttccagcgaggcccggcgccggagttcagcgccagcccccctgcgtgcctgtacatgggccgccagcccccgccgccgccgccgcacccgttccctggcgccctgggcgcgctggagcagggcagccccccggacatctccccgtacgaggtgccccccctcgccgacgaccccgcggtggcgcaccttcaccaccacctcccggctcagctcgcgctcccccacccgcccgccgggcccttcccggagggagccgagccgggcgtcctggaggagcccaaccgcgtccagctgcctttcccatggatgaagtctaccaaagctcacgcgtggaaaggccagtgggcaggtaagcctggctccccacccctttctcctttccggttctcacccggccgccttacctccaagcgctcccaggagccttctctctgttcccggcgccttggattatcccgggtcggactaaactacatcagggagctaccgagcccatccctcacagcagtgcttctctagtccagtttgaagcatctttcccacccagctctcctgggagtgtacactccttccttccctgttcgctgagcccatcttcgccccaggagcccgcgctcccagcgccatccttagagagccgaggctgagtcctgctcagggcttcggacactacagatcctcctccagcaggggatccgggaacccaggactccttggtagtgcacatcgaggaagccgagtagggacatgggtgcctcggacccaggccccagatcgccttcggagccccggagcccctcacttcccgcgcttcgttaaggaagggcaggcatctaggggcgccaggtaggtgcagaaaggcagggagggaaaggaaactgcacccaacccagcagtgtccggctgccctggttgtggaaacaggataggagtaaagaggaaggggctggggcaaggcgggggctcaccgcgaggctgaaagccggcctctcaacgtcagagcctggcagctaggagagcaatctgagaagcgaattcgtttttcaccaaccgaaagcaattgaagctgtctccccgcaccgcttcccaggaagtaatttttcaggagatgggcgctccctgcctagctggtggggaggcgagaggcctggttcctgcggccctccgcgccggcagagaacagaaggtctttcccggagccgggagccggaggcacggggtagcccccgggtcctttgcggccccgcgcgagcggcaagttccggcgcggcctgtgtcgtcgccgctactcactgtcatcgctgccgtgcctcagccacttctggtcacacctgcaccgcaaatagttgccttttcctttcaactggcagccgggagtagggggaagcagctcgagccggcgtcccccggcccaccccgaaaatcctcagcgcccatctgcggggtctggccagccctgcctgacactgaccccaggcgcagccaggaggggctttgtgcgggagagggagggggaccccagcttgcctggggtccacgggactctcttcttcctagttcactttcttgctaaggcgaaggtcctgaggcaggacgagggctgaactgcgctgcaatcgtccccacctccagcgaaacccagttgacaggggcgccagaagctgccgcggcgcctctgcaaatttatccagctcgcgcagcccgggccaaaggccttgaagtctccggaaatgcggggttcttaggaggcgggaggacagtccctcgaacaaaggtggggggctcctcgtcctcacccagttttcttccagggctgcctcccctccagacctctcttctggcctcctaggccctcggagctcctgctttcccaccctgggccttcctcaggaaatgggcgacatcagggtcccgaaagaggatttgtgaggtggagtaacttccctatcccaacccaaggggtgatacctctgctctggaggacttgggcttaggctgacccaagaagccagaaagtaaaaccagaaggcaaatcagcagccttggcgagggttcggggacccaaggagggcgacactctcgggctggagttggccccaggcctttgctggcgccctctaacccgctgcatgctcgactctcggggaaggagacgacctcccctctcttcccctggaagccgtctgcggggccggctgctatccccgcgttcctctaggggaaacttcgatggagccgaaattcaaaaattgcaaacccacctgcccctgggaagagcgaagtgacaaaagggctctcactggcagtacgaatctgaatgctaatgacaacagaggttttgaaaaacattgacccccaaatgcttcagcagcgctgtccagctggcacctaaactgcatcactctgcgccttggggaagggcccaggcttggcgaccttgaccttttcccaccatcctcaacctccacccctgccgcgtcgcgctgagcacaggtcccccgggaatagtgcaccccaggaagtctctccctgagcagtctctcgcagggacttcacgaagccctctcgcagggactatacgaagcccgcagcctaaggcaggaacccagagacatgtcggtttaatgtaaaaactttggagagcctttcaaaatgtttattgaaggcccgtctcgcttctctcccaggcgtgggatgccaggtagattcggggatgcccccagggagtagaactctccctggactagggtttgagcctctgcttcagcttctggcgcctcttctcgacctggggggaaacccagtagggttccatcgcgaaattaaacccgcccccaacacacacacactcgcctttcaattccttaaggcttagccaacattcacaggagaaatgtcccctgcctttgctctaagacaagcctctccccggaactttggtggaacttcccgcgccagcgtccacagcctgggtgcagtcagtattttccacagaaaagaaaagattgggacctggctgagcgcagcggcaaacagtgaatgtgggtctccaacctcctgggccagggcgtcctgttgcctcttggagacacgagaggcttgtttctgcaccactaccacctcctccgtagggctgtcggttctgcagctgggctagggcccctgtgtctcccctcaacacctctgagggcatttgggatccagggcgtagagtctggagctgccagagttctgccctggccaacgtgacccccagaacaatattccttcacttcgcgggcagaagtccggctgaagttaaaacaattatggagaatttgctggctctcaggttgggactaattacgatataactatagagagaggaaacacatggtcagatataacaaaatgtgtcacagtctccattagcacaaagattttcaaactgcaggttgcacccattcgcaggtcataaaatcaatttactaggttgagattagtattttttaaacgaaatagcagataatggagagaaaagtagatagcatcatacgtggtaaacgtttgttttatgtccttaagatttgtcagtataactgacctgcagtgtccgtgtgtgaactacacaacgatccgaaatgtatttctcacatttgtgggtcaccatcaggaggttttttttagccctggattaaaggcgttttattgcctttgtaggatccagccggtttaactattatatacatttaaatcaacatcaatcagttgattaacaccgattatatgagcgcattcaaggaccactcattggcagagccaagcttaggctcacggcgagagctgactcgagtttggtctccaataaaaaggctatctttattaggaagggcttgagttactagggaagagcttcgcgcgcctacactaggcgctgaaatgggatgctggggcttggtggctccggcgggagcagctggtagggctagggctccctggccccccttgaaggggttgggctgcgtgggtgggggctgtgcggggctccgggggccacactcacgccctgtgtcgcccgcaggcggcgcctacgctgcggagccggaggagaacaagcggacgcgcacggcctacacgcgcgcacagctgctagagctggagaaggagttcctattcaacaagtacatctcacggccgcgccgggtggagctggctgtcatgttgaacttgaccgagagacacatcaagatctggttccaaaaccgccgcatgaagtggaaaaaggaggaggacaagaagcgcggcggcgggacagctgtcgggggtggcggggtcgcggagcctgagcaggactgcgccgtgacctccggcgaggagcttctggcgctgccgccgccgccgccccccggaggtgctgtgccgcccgctgcccccgttgccgcccgagagggccgcctgccgcctggccttagcgcgtcgccacagccctccagcgtcgcgcctcggcggccgcaggaaccacgatgagaggcaggagctgctcctggctgaggggcttcaaccactcgccgaggaggagcagagggcctaggaggaccccgggcgtggaccacccgccctggcagttgaatggggcggcaattgcggggcccaccttagaccgaaggggaaaacccgctctctcaggcgcatgtgccagttggggccccgcgggtagatgccggcaggccttccggaagaaaaagagccattggtttttgtagtattggggccctcttttagtgatactggattggcgttgtttgtggctgttgcgcacatccctgccctcctacagcactccaccttgggacctgtttagagaagccggctcttcaaagacaatggaaactgtaccatacacattggaaggctccctaacacacacagcggggaagctgggccgagtaccttaatctgccataaagccattcttactcgggcgacccctttaagtttagaaataattgaaaggaaatgtttgagttttcaaagatcccgtgaaattgatgccagtggaatacagtgagtcctcctcttcctcctcctcctcttccccctccccttcctcctcctcctcttcttttccctcctcttcctcttcctcctgctctcctttcctccccctcctcttttccctcctcttcctcttcctcctgctctcctttcctccccctcctctttctcctcctcctcctcttcttccccctcctctccctcctcctcttcttccccctcctctccctcctcctcttcttctccctcctcttcctcttcctcctcttccacgtgctctcctttcctccccctcctcttgctccccttcttccccgtcctcttcctcctcctcctcttcttctccctcctcttcctcctcctctttcttcctgacctctttctttctcctcctcctccttctacctccccttctcatccctcctcttcctcttctctagctgcacacttcactactgcacatcttataacttgcacccctttcttctgaggaagagaacatcttgcaaggcagggcgagcagcggcagggctggcttaggagcagtgcaagagtccctgtgctccagttccacactgctggcagggaaggcaaggggggacgggcctggatctgggggtgagggagaaagatggacccctgggtgaccactaaaccaaagatattcggaactttctatttaggatgtggacgtaattcctgttccgaggtagaggctgtgctgaagacaagcacagtggcctggtgcgccttggaaaccaacaactattcacgagccagtatgaccttcacatctttagaaattatgaaaacgtatgtgattggagggtttggaaaaccagttatcttatttaacattttaaaaattacctaacagttatttacaaacaggtctgtgcatcccaggtctgtcttcttttcaaggtctgggccttgtgctcgggttatgtttgtgggaaatgcttaataaatactgataatatgggaagagatgaaaactgattctcctcactttgtttcaaaccttt

SEQ ID NO: 12 - DNA, 7218 bp, Homo sapiens
tggaaaatgcagcgtttttatttctttttctaaatatgtaactcttcctccacttccccctctcctgcttgccttatttcaattgcaagcagaagagagtgagtgttctctgccggcaaactccgccagggtcccggcccgtagagagtcgtcaagggtctggaacccccgtgccaacacctgcccctgcttcgcagccccaagaggaaggccgcgtctttccccctcgctgtattgggaagctacgttccgggctggccaaatgggccccaattttccaaaacccaaatttgtaatacccttcaattttttaaaaaaaagaatttaaaaaagtctctgtgaatgcttcagaagttaccgtttacaccccagaagtacttgcagcacatccacaagtaaaaacacacaacgaatgccagagtttcgtgtgttttttaaccgacatctttgtggctgtgaacaaacttcataaataaaatagaatcaaatgcttctgacctagagagctgggtctgcaaactttttttttatcgtattccgcaacagttaaataaaaaattaaaaactcaacatgtctccttgtaaactacatcaattaacaaacacactatgtccattatcaaatataatagaaaaaatataggaaaatagaaaatagaaaaatataggaaaatagaaacttttaagccacggtgaaaatgtttctataaatgagtggttctaatgttttcgtgagcgcccattttggggagcaccgccagctgcccgttcaggagtgtgcagcaaactcagctgagagagaaaattggaacaaaagcaggtgctcgcgggtacctgggcctagcctcttagtgcggccagccaggccaatcacggcccccggctgaaccacgtggggccccgcggagcctatggtgcggcggccggcccgccggtccgcgctggctgtgggttccctctgagatcagtgcggagctgtcaaagcgagcaggggtggcgccgggagtgggaacgccacacagtgccaaatccccggctccagctcccgactcccggctcccggctcccggctcccggtgcccaatcccgggccgcagccatgaacggcgaggagcagtactacgcggccacgcagctttacaaggacccatgcgcgttccagcgaggcccggcgccggagttcagcgccagcccccctgcgtgcctgtacatgggccgccagcccccgccgccgccgccgcacccgttccctggcgccctgggcgcgctggagcagggcagccccccggacatctccccgtacgaggtgccccccctcgccgacgaccccgcggtggcgcaccttcaccaccacctcccggctcagctcgcgctcccccacccgcccgccgggcccttcccggagggagccgagccgggcgtcctggaggagcccaaccgcgtccagctgcctttcccatggatgaagtctaccaaagctcacgcgtggaaaggccagtgggcaggtaagcctggctccccacccctttctcctttccggttctcacccggccgccttacctccaagcgctcccaggagccttctctctgttcccggcgccttggattatcccgggtcggactaaactacatcagggagctaccgagcccatccctcacagcagtgcttctctagtccagtttgaagcatctttcccacccagctctcctgggagtgtacactccttccttccctgttcgctgagcccatcttcgccccaggagcccgcgctcccagcgccatccttagagagccgaggctgagtcctgctcagggcttcggacactacagatcctcctccagcaggggatccgggaacccaggactccttggtagtgcacatcgaggaagccgagtagggacatgggtgcctcggacccaggccccagatcgccttcggagccccggagcccctcacttcccgcgcttcgttaaggaagggcaggcatctaggggcgccaggtaggtgcagaaaggcagggagggaaaggaaactgcacccaacccagcagtgtccggctgccctggttgtggaaacaggataggagtaaagaggaaggggctggggcaaggcgggggctcaccgcgaggctgaaagccggcctctcaacgtcagagcctggcagctaggagagcaatctgagaagcgaattcgtttttcaccaaccgaaagcaattgaagctgtctccccgcaccgcttcccaggaagtaatttttcaggagatgggcgctccctgcctagctggtggggaggcgagaggcctggttcctgcggccctccgcgccggcagagaacagaaggtctttcccggagccgggagccggaggcacggggtagcccccgggtcctttgcggccccgcgcgagcggcaagttccggcgcggcctgtgtcgtcgccgctactcactgtcatcgctgccgtgcctcagccacttctggtcacacctgcaccgcaaatagttgccttttcctttcaactggcagccgggagtagggggaagcagctcgagccggcgtcccccggcccaccccgaaaatcctcagcgcccatctgcggggtctggccagccctgcctgacactgaccccaggcgcagccaggaggggctttgtgcgggagagggagggggaccccagcttgcctggggtccacgggactctcttcttcctagttcactttcttgctaaggcgaaggtcctgaggcaggacgagggctgaactgcgctgcaatcgtccccacctccagcgaaacccagttgacaggggcgccagaagctgccgcggcgcctctgcaaatttatccagctcgcgcagcccgggccaaaggccttgaagtctccggaaatgcggggttcttaggaggcgggaggacagtccctcgaacaaaggtggggggctcctcgtcctcacccagttttcttccagggctgcctcccctccagacctctcttctggcctcctaggccctcggagctcctgctttcccaccctgggccttcctcaggaaatgggcgacatcagggtcccgaaagaggatttgtgaggtggagtaacttccctatcccaacccaaggggtgatacctctgctctggaggacttgggcttaggctgacccaagaagccagaaagtaaaaccagaaggcaaatcagcagccttggcgagggttcggggacccaaggagggcgacactctcgggctggagttggccccaggcctttgctggcgccctctaacccgctgcatgctcgactctcggggaaggagacgacctcccctctcttcccctggaagccgtctgcggggccggctgctatccccgcgttcctctaggggaaacttcgatggagccgaaattcaaaaattgcaaacccacctgcccctgggaagagcgaagtgacaaaagggctctcactggcagtacgaatctgaatgctaatgacaacagaggttttgaaaaacattgacccccaaatgcttcagcagcgctgtccagctggcacctaaactgcatcactctgcgccttggggaagggcccaggcttggcgaccttgaccttttcccaccatcctcaacctccacccctgccgcgtcgcgctgagcacaggtcccccgggaatagtgcaccccaggaagtctctccctgagcagtctctcgcagggacttcacgaagccctctcgcagggactatacgaagcccgcagcctaaggcaggaacccagagacatgtcggtttaatgtaaaaactttggagagcctttcaaaatgtttattgaaggcccgtctcgcttctctcccaggcgtgggatgccaggtagattcggggatgcccccagggagtagaactctccctggactagggtttgagcctctgcttcagcttctggcgcctcttctcgacctggggggaaacccagtagggttccatcgcgaaattaaacccgcccccaacacacacacactcgcctttcaattccttaaggcttagccaacattcacaggagaaatgtcccctgcctttgctctaagacaagcctctccccggaactttggtggaacttcccgcgccagcgtccacagcctgggtgcagtcagtattttccacagaaaagaaaagattgggacctggctgagcgcagcggcaaacagtgaatgtgggtctccaacctcctgggccagggcgtcctgttgcctcttggagacacgagaggcttgtttctgcaccactaccacctcctccgtagggctgtcggttctgcagctgggctagggcccctgtgtctcccctcaacacctctgagggcatttgggatccagggcgtagagtctggagctgccagagttctgccctggccaacgtgacccccagaacaatattccttcacttcgcgggcagaagtctggctgaagttaaaacaattatggagaatttgctggctctcaggttgggactaattacgatataactatagagagaggaaacacatggtcagatataacaaaatgtgtcacagtctccattagcacaaagattttcaaactgcaggttgcacccattcgcaggtcataaaatcaatttactaggttgagattagtattttttaaacgaaatagcagataatggagagaaaagtagatagcatcatacgtggtaaacgtttgttttatgtccttaagatttgtcagtataactgacctgcagtgtccgtgtgtgaactacacaacgatccgaaatgtatttctcacatttgtgggtcaccatcaggaggttttttttagccctggattaaaggcgttttattgcctttgtaggatccagccggtttaactattatatacatttaaatcaacatcaatcagttgattaacaccgattatatgagcgcattcaaggaccactcattggcagagccaagcttaggctcacggcgagagctgactcgagtttggtctccaataaaaaggctatctttattaggaagggcttgagttactagggaagagcttcgcgcgcctacactaggcgctgaaatgggatgctggggcttggtggctccggcgggagcagctggtagggctagggctccctggccccccttgaaggggttgggctgcgtgggtgggggctgtgcggggctccgggggccacactcacgccctgtgtcgcccgcaggcggcgcctacgctgcggagccggaggagaacaagcggacgcgcacggcctacacgcgcgcacagctgctagagctggagaaggagttcctattcaacaagtacatctcacggccgcgccgggtggagctggctgtcatgttgaacttgaccgagagacacatcaagatctggttccaaaaccgccgcatgaagtggaaaaaggaggaggacaagaagcgcggcggcgggacagctgtcgggggtggcggggtcgcggagcctgagcaggactgcgccgtgacctccggcgaggagcttctggcgctgccgccgccgccgccccccggaggtgctgtgccgcccgctgcccccgttgccgcccgagagggccgcctgccgcctggccttagcgcgtcgccacagccctccagcgtcgcgcctcggcggccgcaggaaccacgatgagaggcaggagctgctcctggctgaggggcttcaaccactcgccgaggaggagcagagggcctaggaggaccccgggcgtggaccacccgccctggcagttgaatggggcggcaattgcggggcccaccttagaccgaaggggaaaacccgctctctcaggcgcatgtgccagttggggccccgcgggtagatgccggcaggccttccggaagaaaaagagccattggtttttgtagtattggggccctcttttagtgatactggattggcgttgtttgtggctgttgcgcacatccctgccctcctacagcactccaccttgggacctgtttagagaagccggctcttcaaagacaatggaaactgtaccatacacattggaaggctccctaacacacacagcggggaagctgggccgagtaccttaatctgccataaagccattcttactcgggcgacccctttaagtttagaaataattgaaaggaaatgtttgagttttcaaagatcccgtgaaattgatgccagtggaatacagtgagtcctcctcttcctcctcctcctcttccccctccccttcctcctcctcctcttcttttccctcctcttcctcttcctcctgctctcctttcctccccctcctcttttccctcctcttcctcttcctcctgctctcctttcctccccctcctctttctcctcctcctcctcttcttccccctcctctccctcctcctcttcttccccctcctctccctcctcctcttcttctccctcctcttcctcttcctcctcttccacgtgctctcctttcctccccctcctcttgctccccttcttccccgtcctcttcctcctcctcctcttcttctccctcctcttcctcctcctctttcttcctgacctctttctttctcctcctcctccttctacctccccttctcatccctcctcttcctcttctctagctgcacacttcactactgcacatcttataacttgcacccctttcttctgaggaagagaacatcttgcaaggcagggcgagcagcggcagggctggcttaggagcagtgcaagagtccctgtgctccagttccacactgctggcagggaaggcaaggggggacgggcctggatctgggggtgagggagaaagatggacccctgggtgaccactaaaccaaagatattcggaactttctatttaggatgtggacgtaattcctgttccgaggtagaggctgtgctgaagacaagcacagtggcctggtgcgccttggaaaccaacaactattcacgagccagtatgaccttcacatctttagaaattatgaaaacgtatgtgattggagggtttggaaaaccagttatcttatttaacattttaaaaattacctaacagttatttacaaacaggtctgtgcatcccaggtctgtcttcttttcaaggtctgggccttgtgctcgggttatgtttgtgggaaatgcttaataaatactgataatatgggaagagatgaaaactgattctcctcactttgtttcaaaccttt

SEQ ID NO: 13 - DNA, 25 bp, Homo sapiens
acgtgacccccagaacaatattcct

SEQ ID NO: 14 - DNA, 24 bp, Homo sapiens
cctgagagccagcaaattctccat

SEQ ID NO: 15 - DNA, 15 bp, Homo sapiens
cagccagacttctgc

SEQ ID NO: 16 - DNA, 14 bp, Homo sapiens
cagccggacttctg

SEQ ID NO: 17 - DNA, 45 bp, Homo sapiens
cttcacttcgcgggcagaagtctggctgaagttaaaacaattatg

SEQ ID NO: 18 - DNA, 45 bp, Homo sapiens
cataattgttttaacttcagccagacttctgcccgcgaagtgaag

(19) United States
(12) Patent Application Publication
(10) Pub. No.: US 2011/0020797 A1
(43) Pub. Date: Jan. 27, 2011

(54) Methods For Identifying Patients With An Increased Likelihood Of Responding To DPP-IV Inhibitors

(75) Inventor: Ranade Koustubh, Princeton, NJ (US)

Correspondence Address: McDonnell Boehnen Hulbert & Berghoff LLP;Bristol-Myers Squibb, 300 South Wacker Drive, Chicago, IL, 60606, US

(73) Assignee: BRISTOL-MYERS SQUIBB COMPANY, Princeton, NJ (US)

(21) Appl. No.: 12/526,320

(22) PCT Filed: Feb. 11, 2008
(86) PCT No.: PCT/US08/53616

§ 371 Date: Mar. 25, 2010

Related U.S. Application Data
(60) Provisional application No. 60/900,395, filed on Feb. 9, 2007.

(51) Int. Cl. C12Q 1/68

(52) U.S. Cl. 435/6

(57) ABSTRACT

The invention provides novel in vitro diagnostic methods for identifying patients who may have an increased likelihood of responding to DPP-IV inhibitor therapy. The invention also provides novel polynucleotides associated with increased responsiveness of a patient to DPP-IV inhibition. Polynucleotide fragments corresponding to the genomic and/or coding regions of these polynucleotides, which comprise at least one polymorphic locus per fragment, are also provided. Allele-specific primers and probes which hybridize to these polymorphic regions, and/or which comprise at least one polymorphic locus are also provided. The polynucleotides, primers, and probes of the present invention are useful in phenotype correlations, medicine, and genetic analysis.

### FIELD OF THE INVENTION

The invention provides novel in vitro diagnostic methods for identifying patients who may have an increased likelihood of responding to DPP-IV inhibitor therapy. The invention also provides novel polynucleotides associated with increased responsiveness of a patient to DPP-IV inhibition. Polynucleotide fragments corresponding to the genomic and/or coding regions of these polynucleotides, which comprise at least one polymorphic locus per fragment, are also provided. Allele-specific primers and probes which hybridize to these polymorphic regions, and/or which comprise at least one polymorphic locus are also provided. The polynucleotides, primers, and probes of the invention are useful in diagnostic methods, phenotype correlations, medicine, and genetic analysis.

### BACKGROUND OF THE INVENTION

Dipeptidyl peptidase IV (DPP-IV) inhibitors interfere with the degradation of incretins like Glucagon-Like Peptide-1 (GLP-1), thereby increasing the amount of insulin secreted by the pancreas. DPP-IV inhibitors are being developed, therefore, to treat type II diabetes.

Maturity Onset Diabetes of the Young (MODY) is characterized by an autosomal dominantly-inherited, early onset form of non-insulin dependent diabetes mellitus. The mean age at time of diagnosis is 23 years, and approximately one-third of patients with MODY develop progressive β-cell failure requiring insulin-replacement therapy (Pearson et al., 2000, Diabet Med. 17:543-545).

CYP3A5 is a cytochrome belonging to the P450 family which is responsible for catalyzing metabolism of numerous structurally diverse exogenous and endogenous molecules. Approximately 55 different CYP genes are present in the human genome and are classified into different families and subfamilies on the basis of sequence homology. The CYP families have arisen through a process of gene duplication and gene conversion. Members of the CYP3A subfamily catalyze oxidative, peroxidative and reductive metabolism of structurally diverse endobiotics, drugs, and protoxic or procarcinogenic molecules (Rendic & DiCarlo, 1997, Drug Metab. Rev. 29: 413-580). The CYP3A members are the most abundant CYPs expressed in human liver and small intestine (Cholerton et al., 1992, Trends Pharmacol. Sci. 13: 434-439, and Shimada et al., 1994, J. Pharmacol. Exp. Ther. 270: 414-423). Substantial interindividual differences in CYP3A expression, exceeding 30-fold in some populations (Watkins, 1995, Hepatology 22: 994-996), contribute greatly to variation in oral bioavailability and systemic clearance of CYP3A substrates, including HIV protease inhibitors, several calcium channel blockers and some cholesterol-lowering drugs (Kuehl et al., 2001, Nature Genetics 27: 383-391).

Human CYP3A activities reflect the heterogeneous expression of at least three CYP3A family members: CYP3A4, CYP3A5 and CYP3A7. The CYP3A genes are adjacent to each other on chromosome band 7q21, but the genes are differentially regulated (Finta & Zaphiropoulos, 2000, Gene 260: 13-23). Single nucleotide polymorphisms (SNPs) in the regulatory sequences of CYP3A are believed to result in regulation of their expression (Kuehl et al., 2001, Nature Genetic 27: 383-391). In particular, analysis of human liver CYP3A5 cDNA revealed that only those people with the CYP3A5*1 allele produce high levels of full-length CYP3A5 mRNA and express CYP3A5. Those individuals with the CYP3A5*3 allele have sequence variability in intron 3 that creates a cryptic splice site, which results in the generation of CYP3A5 exon 3B, resulting in this allele encoding an aberrantly spliced mRNA with a premature stop codon. This helps explain the molecular defect responsible for one of the most common polymorphisms in drug-metabolizing enzymes (Kuehl et al., Id.).

Insulin Promotor Factor-1 (IPF-1, also known as Pancreatic Doudenal Homeobox Protein-1 [PDX-1], and STF-1) is a transcription factor that is essential for normal development of the pancreas (Habener, 2002, Drug News Perspect 15:491-497). IPF-1 also contributes to glucose-dependent expression of insulin, the glucose transporter GLUT2, and glucokinase in pancreatic β-cells, and thus plays a critical role in normal function of the endocrine pancreas. A number of missense and codon insertion mutations in the IPF-1 gene have been associated with Maturity Onset Diabetes of the Young Type 4 (MODY4) in kindreds from the United States, Great Britain, and Europe (Habener, 2002, Id.; Gragnoli et al, 2006, Metab Clin Exper. 54:983-988). These references also report that functional analyses of MODY4-associated variants of IPF-1 demonstrate reduced binding of the gene product to the promoter sequence of the insulin gene and reduced transactivation of insulin. There is experimental evidence that adult pancreatic β-cells undergo spontaneous apoptosis and that regeneration of β-cells from a pool of progenitor cells is essential to maintenance of normal β-cell mass in the adult pancreas. Expression of IPF-1 appears to be sufficient to drive differentiation of progenitor cells into functioning β-cells, thus implicating IPF-1 in the maintenance of normal β-cell mass and function (Habener, 2002, Drug News Perspect 15:491-497; Nakjima-Nagata et al., 2004, BBRC 8:625-630). Glucagon-Like Peptide-1 (GLP-1), a key substrate for DPP-IV, is particularly effective in stimulating the expression of IPF-1 and inducing the differentiation of progenitor cells into functioning β-cells (Habener 2002, Id.).

There is a need in the art to identify genetic polymorphisms of genes known to be associated with pancreatic function that can predict patient responsiveness to DPP-IV inhibitors, to improve treatment and avoid unnecessary side-effects or ineffective treatment of diabetes or other metabolic diseases and disorders.

### SUMMARY OF THE INVENTION

The present invention provides genetic polymorphisms in the CYP3A5 and IPF-1 genes which can be used to identify those patients most likely to respond to DPP-IV inhibition. Such polymorphisms may genetically predispose certain individuals to increased responsiveness to DPP-IV inhibition. Accordingly, genotypes of such polymorphisms can be predicative of an individual's likelihood of responding to DPP-IV inhibition and can be used to establish a treatment regimen optimized for each individual.

The invention further relates to methods of determining whether an individual has an increased likelihood of having a favorable response to the administration of a pharmaceutically acceptable level of a DPP-IV inhibitor comprising the step of determining whether the individual harbors either the reference or variant allele of either the CYP3A5 or IPF-1 gene, wherein an individual harboring the variant allele has a higher likelihood of a favorable response to an administered DPP-IV inhibitor relative to an individual harboring the reference allele. The methods can be used to determine whether the individual may respond to a lower level of administered DPP-IV inhibitor.

The invention also relates to nucleic acid molecules comprising at least one single nucleotide polymorphism within the CYP3A5 or IPF-1 genomic sequence at a specific polymorphic locus. In certain embodiments, the invention relates to the variant allele of the CYP3A5 or IPF-1 gene or polynucleotide having at least one single nucleotide polymorphism, which variant allele differs from a reference allele by one nucleotide at the site(s) identified in FIGS. 1A-L or FIGS. 2A-L for the CYP3A5 gene, or as identified in FIGS. 4A-D or FIGS. 5A-D for the IPF1 gene, or elsewhere herein. The complementary sequences of each of these nucleic acid molecules are also provided. The nucleic acid molecules of the invention can comprise DNA or RNA, can be double- or single-stranded, and can comprise fragments thereof. The fragments can be about 5 to about 100 nucleotides in length including, for example, about 5 to about 10 nucleotides, about 5 to about 15 nucleotides, about 10 to about 20 nucleotides, about 15 to about 25 nucleotides, about 10 to about 30 nucleotides, about 10 to about 40 nucleotides, about 10 to about 50 nucleotides, or about 50 to about 100 nucleotides long, and preferably comprise at least one polymorphic allele.

In other embodiments, the invention relates to the reference allele of the CYP3A5 or IPF-1 gene or polynucleotide having at least one polymorphic locus, in which said reference allele differs from a variant allele by one nucleotide at the polymorphic site(s) identified in FIGS. 1A-L or FIGS. 2A-L for the CYP3A5 gene, or as identified in FIGS. 4A-D or FIGS. 5A-D for the IPF1 gene, or elsewhere herein. The complementary sequences of each of these nucleic acid molecules are also provided. The nucleic acid molecules can comprise DNA or RNA, can be double- or single-stranded, and can comprise fragments thereof. The fragments can be about 5 to about 100 nucleotides in length including, for example, about 5 to about 10 nucleotides, about 5 to about 15 nucleotides, about 10 to about 20 nucleotides, about 15 to about 25 nucleotides, about 10 to about 30 nucleotides, about 10 to about 40 nucleotides, about 10 to about 50 nucleotides, or about 50 to about 100 nucleotides long, and preferably comprise at least one polymorphic allele.

The invention further provides variant and reference allele-specific oligonucleotides that hybridize to a nucleic acid molecule comprising at least one polymorphic locus, in addition to the complement of said oligonucleotide. These oligonucleotides can be probes or primers, for example, oligonucleotide primers for amplifying a nucleic acid sequence across a polymorphic locus and oligonucleotide primers for sequencing the amplified nucleic acid sequences or other sequences.

The invention further provides oligonucleotides that can be used to amplify a portion of either the variant or reference sequences comprising at least one polymorphic locus, in addition to providing oligonucleotides that can be used to sequence said amplified sequence. The invention further provides a method of analyzing a nucleic acid from a DNA or RNA sample using said amplification and sequencing primers to determine whether the sample contains the reference or variant nucleotide (allele) at the polymorphic locus, comprising the steps of amplifying a sequence using appropriate oligonucleotide primers for amplifying across a polymorphic locus, and sequencing the resulting amplified sequence product using appropriate sequencing primers to sequence the amplified product to determine whether the variant or reference nucleotide is present at the polymorphic locus.

The invention further provides methods for analyzing a nucleic acid from patient sample(s) using said amplification and sequencing primers to determine whether said sample(s) contain the reference or variant nucleotide (allele) at the polymorphic locus in an effort to identify patient populations with an increased likelihood of a favorable response to the administration of a pharmaceutically acceptable amount of a DPP-IV inhibitor, comprising the steps of amplifying a nucleic acid sequence using appropriate oligonucleotide primers for amplifying across a polymorphic locus, and sequencing the resulting amplified sequence product using appropriate sequencing primers to sequence said product to determine whether the variant or reference nucleotide is present at the polymorphic locus.

The invention further provides oligonucleotides that can be used to genotype patient sample(s) to assess whether said sample(s) contain the reference or variant nucleotide (allele) at the polymorphic site(s). The invention provides methods of using the oligonucleotides to genotype a patient sample to determine whether said sample contains the reference or variant nucleotide (allele) at the polymorphic locus. An embodiment of the method comprises the steps of amplifying a nucleic acid sequence using appropriate oligonucleotide primers for amplifying across a polymorphic locus, and subjecting the product of said amplification to an analysis assay, such as a genetic bit analysis (GBA) reaction.

The invention also provides methods of using oligonucleotides that can be used to genotype patient sample(s) to identify individual(s) with an increased likelihood of a favorable response to the administration of a pharmaceutically acceptable amount of a DPP-IV inhibitor to determine whether said sample(s) contains the reference or variant nucleotide (allele) at one or more polymorphic loci. An embodiment of the method comprises the steps of amplifying a nucleic acid sequence using appropriate oligonucleotide primers for amplifying across a polymorphic locus, and subjecting the product of said amplification to an analysis assay, such as a genetic bit analysis (GBA) reaction, and optionally determining the statistical association between either the reference or variant allele at the polymorphic site(s) to an increased likelihood of a favorable response to the administration of a pharmaceutically acceptable amount of a DPP-IV inhibitor.

The invention provides a method of using oligonucleotides that can be used to genotype patient sample(s) to identify ethnic population(s), in one particular embodiment Hispanic populations, with an increased likelihood of a favorable response to the administration of a pharmaceutically acceptable amount of a DPP-IV inhibitor to assess whether said sample(s) contains the reference or variant nucleotide (allele) at one or more polymorphic loci comprising the steps of amplifying a nucleic acid sequence using appropriate oligonucleotide primers for amplifying across a polymorphic locus, and subjecting the product of said amplification to an analysis assay, such as a genetic bit analysis (GBA) reaction, and optionally determining the statistical association between either the reference or variant allele at the polymorphic site(s) to an increased likelihood of a favorable response to the administration of a pharmaceutically acceptable amount of a DPP-IV inhibitor.

The polynucleotides and oligonucleotides provided herein can be used to analyze a nucleic acid from one or more individuals to determine whether the reference or variant nucleotide is present at any one, or more, of the polymorphic sites identified in FIGS. 1A-L or FIGS. 2A-L for the CYP3A5 gene, or as identified in FIGS. 4A-D or FIGS. 5A-D for the IPF1 gene, or elsewhere herein. Optionally, a set of nucleotides occupying a set of the polymorphic loci shown in FIGS. 1A-L or FIGS. 2A-L for the CYP3A5 gene, or as identified in FIGS. 4A-D or FIGS. 5A-D for the IPF-1 gene, or elsewhere herein, is determined. This type of analysis can be performed on a number of individuals, who are also tested (previously, concurrently or subsequently) for an increased likelihood of a favorable response to the administration of a pharmaceutically acceptable amount of a DPP-IV inhibitor. The increased likelihood of a favorable response to the administration of a pharmaceutically acceptable amount of a DPP-IV inhibitor phenotype is then correlated with said nucleotide or set of nucleotides present at the polymorphic locus or loci in the individuals tested.

The invention thus further relates to a method of identifying an increased likelihood of a favorable response to the administration of a pharmaceutically acceptable amount of a DPP-IV inhibitor associated with a particular genotype. The method comprises obtaining a nucleic acid sample from an individual and determining the identity of one or more nucleotides at specific polymorphic loci of nucleic acid molecules described herein, wherein the presence of a particular nucleotide at that site is correlated with an increased likelihood of a favorable response to the administration of a pharmaceutically acceptable amount of a DPP-IV inhibitor, thereby identifying an increased likelihood of a favorable response to the administration of a pharmaceutically acceptable amount of a DPP-IV inhibitor in the individual.

The invention further relates to polynucleotides having one or more polymorphic loci comprising one or more variant alleles. The invention also relates to said polynucleotides lacking a start codon. The invention further relates to polynucleotides of the present invention containing one or more variant alleles wherein said polynucleotides encode a polypeptide of the present invention. The invention relates to polypeptides of the present invention containing one or more variant amino acids encoded by one or more variant alleles.

The present invention relates to antisense oligonucleotides capable of hybridizing to the polynucleotides of the present invention. Preferably, such antisense oligonucleotides are capable of discriminating between the reference or variant allele of the polynucleotide, preferably at one or more polymorphic sites of said polynucleotide.

The present invention relates to siRNA or RNAi oligonucleotides capable of hybridizing to the polynucleotides of the present invention. Preferably, such siRNA or RNAi oligonucleotides are capable of discriminating between the reference or variant allele of the polynucleotide, preferably at one or more polymorphic sites of said polynucleotide.

The present invention also relates to zinc finger proteins capable of binding to the polynucleotides of the present invention. Preferably, such zinc finger proteins are capable of discriminating between the reference or variant allele of the polynucleotide, preferably at one or more polymorphic sites of said polynucleotide.

The present invention also relates to recombinant vectors, which include the isolated nucleic acid molecules of the present invention, and to host cells containing the recombinant vectors, as well as to methods of making such vectors and host cells, in addition to their use in the production of polypeptides or peptides provided herein using recombinant techniques. Synthetic methods for producing the polypeptides and polynucleotides of the present invention are provided. Also provided are diagnostic methods for detecting diseases, disorders, and/or conditions related to the polypeptides and polynucleotides provided herein, and therapeutic methods for treating such diseases, disorders, and/or conditions. The invention further relates to screening methods for identifying binding partners of the polypeptides.

The invention relates to a method of analyzing one or more nucleic acid samples comprising the step of determining the nucleic acid sequence from one or more samples at one or more polymorphic loci in the human CYP3A5 or IPF-1 gene, wherein the presence of the reference allele at said one or more polymorphic loci is indicative of a decreased likelihood of a favorable response to the administration of a pharmaceutically acceptable amount of a DPP-IV inhibitor.

The invention relates to a method of analyzing one or more nucleic acid samples comprising the step of determining the nucleic acid sequence from one or more samples to determine the isoform present for the human CYP3A5 gene, wherein the presence of isoform 1 (reference allele) is indicative of a decreased likelihood of a favorable response to the administration of a pharmaceutically acceptable amount of a DPP-IV inhibitor.

The invention relates to a method of analyzing one or more nucleic acid samples comprising the step of determining the nucleic acid sequence from one or more samples at one or more polymorphic loci in the human CYP3A5 or IPF-1 gene, wherein the presence of the variant allele at said one or more polymorphic loci is indicative of an increased likelihood of a favorable response to the administration of a pharmaceutically acceptable amount of a DPP-IV inhibitor.

The invention relates to a method of analyzing one or more nucleic acid samples comprising the step of determining the nucleic acid sequence from one or more samples to determine the isoform present for the human CYP3A5 gene, wherein the presence of isoform 3 is indicative of an increased likelihood of a favorable response to the administration of a pharmaceutically acceptable amount of a DPP-IV inhibitor.

The invention further relates to a method of constructing haplotypes using the isolated nucleic acids referred to in FIGS. 1A-L or FIGS. 2A-L for the CYP3A5 gene, or as identified in FIGS. 4A-D or FIGS. 5A-D for the IPF-1 gene, or elsewhere herein, comprising the step of grouping at least two of the isolated nucleic acids.

The invention further relates to a method of constructing haplotypes further comprising the step of using said haplotypes to identify an individual with an increased likelihood of a favorable response to the administration of a pharmaceutically acceptable amount of a DPP-IV inhibitor phenotype, and correlating the presence of such a phenotype with said haplotype.

The invention further relates to a library of nucleic acids, each of which comprises one or more polymorphic positions within a gene encoding the human CYP3A5 or IPF-1 protein, wherein said polymorphic positions are selected from the polymorphic positions provided in FIGS. 1A-L or FIGS. 2A-L for the CYP3A5 gene or the polymorphic positions identified in FIGS. 4A-D or FIGS. 5A-D for the IPF-1 gene.

The invention further relates to a library of nucleic acids, wherein the sequence at said aforementioned polymorphic positions is selected from the group consisting of the polymorphic position identified in FIGS. 1A-L or FIGS. 2A-L for the CYP3A5 gene or as identified in FIGS. 4A-D or FIGS. 5A-D for the IPF-1 gene, or elsewhere herein, the complementary sequence of said sequences, and/or fragments of said sequences.

The invention further relates to a kit for identifying an individual with an increased likelihood of a favorable response to the administration of a pharmaceutically acceptable amount of a DPP-IV inhibitor, wherein said kit comprises oligonucleotides capable of identifying the nucleotide residing at one or more polymorphic loci of the human CYP3A5 or IPF-1 gene, wherein the presence of the variant allele at said one or more polymorphic loci is indicative of an increased likelihood of a favorable response to the administration of a pharmaceutically acceptable amount of a DPP-IV inhibitor and the presence of the reference allele at said one or more polymorphic loci is indicative of a decreased likelihood of a favorable response to the administration of a pharmaceutically acceptable amount of a DPP-IV inhibitor. In one embodiment, the kit comprises oligonucleotides primers that can amplify a portion of the variant and/or reference sequences comprising at least one polymorphic locus of the human CYP3A5 or IPF-1 gene, for example, oligonucleotide primers that amplify sequence across the polymorphic locus. In another embodiment, the kit additionally comprises oligonucleotides that can be used to sequence said amplified sequence.

The invention further relates to a kit for identifying an individual with an increased likelihood of a favorable response to the administration of a pharmaceutically acceptable amount of a DPP-IV inhibitor, wherein said kit comprises oligonucleotides capable of identifying the nucleotide residing at one or more polymorphic loci of the human CYP3A5 or IPF-1 gene, wherein the presence of the variant allele at said one or more polymorphic loci is indicative of an increased likelihood of a favorable response to the administration of a pharmaceutically acceptable amount of a DPP-IV inhibitor and the presence of the reference allele at said one or more polymorphic loci is indicative of an decreased likelihood of a favorable response to the administration of a pharmaceutically acceptable amount of a DPP-IV inhibitor, and wherein said oligonucleotides hybridize immediately adjacent to said one or more polymorphic positions or wherein said oligonucleotides hybridize to said polymorphic positions such that the central position of the primer aligns with the polymorphic position of said gene. For example, in specific embodiments, the kit comprises the oligonucleotides of SEQ ID NOs: 3-6 and/or the oligonucleotides of SEQ ID NOs: 13-16.

The invention further relates to a method for determining the likelihood that an individual will have a favorable response to the administration of a pharmaceutically acceptable amount of a DPP-IV inhibitor comprising the step of determining the nucleotide present within at least one or more nucleic acid sample(s) from an individual to be assessed at one or more polymorphic position(s) of the human CYP3A5 gene sequence selected from SEQ ID NO:1 and/or SEQ ID NO:2, wherein the presence of the reference nucleotide at the one or more polymorphic position(s) indicates that the individual has a decreased likelihood of a favorable response to the administration of a pharmaceutically acceptable amount of a DPP-IV inhibitor as compared to an individual having the variant allele at said polymorphic position(s).

The invention further relates to a method for determining the likelihood that an individual will have a favorable response to the administration of a pharmaceutically acceptable amount of a DPP-IV inhibitor comprising the step of determining the nucleotide present within at least one or more nucleic acid sample(s) from an individual to be assessed at one or more polymorphic position(s) of the human CYP3A5 gene sequence selected from SEQ ID NO:1 and/or SEQ ID NO:2, wherein the presence of the variant nucleotide at the one or more polymorphic position(s) indicates that the individual has an increased likelihood of a favorable response to the administration of a pharmaceutically acceptable amount of a DPP-IV inhibitor as compared to an individual having the reference allele at said polymorphic position(s).

The invention further relates to a method for determining the likelihood that an individual will have a favorable response to the administration of a pharmaceutically acceptable amount of a DPP-IV inhibitor comprising the step of determining the nucleotide present within at least one or more nucleic acid sample(s) from an individual to be assessed at one or more polymorphic position(s) of the human CYP3A5 gene sequence selected from nucleotide position 7068 of SEQ ID NO:1 and/or SEQ ID NO:2, wherein the presence of the reference nucleotide at nucleotide position 7068 indicates that the individual has a decreased likelihood of a favorable response to the administration of a pharmaceutically acceptable amount of a DPP-IV inhibitor as compared to an individual having the variant allele at said polymorphic position(s).

The invention further relates to a method for determining the likelihood that an individual will have a favorable response to the administration of a pharmaceutically acceptable amount of a DPP-IV inhibitor comprising the step of determining the nucleotide present within at least one or more nucleic acid sample(s) from an individual to be assessed at one or more polymorphic position(s) of the human CYP3A5 gene sequence selected from nucleotide position 7068 of SEQ ID NO:1 and/or SEQ ID NO: 2, wherein the presence of the variant nucleotide at nucleotide position 7068 indicates that the individual has an increased likelihood of a favorable response to the administration of a pharmaceutically acceptable amount of a DPP-IV inhibitor as compared to an individual having the reference allele at said polymorphic position(s).

The invention further relates to a method for determining the likelihood that an individual will have a favorable response to the administration of a pharmaceutically acceptable amount of a DPP-IV inhibitor comprising the step of determining the nucleotide present within at least one or more nucleic acid sample(s) from an individual to be assessed at one or more polymorphic position(s) of the human IPF-1 gene sequence selected from SEQ ID NO:11 and/or SEQ ID NO:12, wherein the presence of the reference nucleotide at the one or more polymorphic position(s) indicates that the individual has a decreased likelihood of a favorable response to the administration of a pharmaceutically acceptable amount of a DPP-IV inhibitor as compared to an individual having the variant allele at said polymorphic position(s).

The invention further relates to a method for determining the likelihood that an individual will have a favorable response to the administration of a pharmaceutically acceptable amount of a DPP-IV inhibitor comprising the step of determining the nucleotide present within at least one or more nucleic acid sample(s) from an individual to be assessed at one or more polymorphic position(s) of the human IPF-1 gene sequence selected from SEQ ID NO:11 and/or SEQ ID NO:12, wherein the presence of the variant nucleotide at the one or more polymorphic position(s) indicates that the individual has an increased likelihood of a favorable response to the administration of a pharmaceutically acceptable amount of a DPP-IV inhibitor as compared to an individual having the reference allele at said polymorphic position(s).

The invention further relates to a method for determining the likelihood that an individual will have a favorable response to the administration of a pharmaceutically acceptable amount of a DPP-IV inhibitor comprising the step of determining the nucleotide present within at least one or more nucleic acid sample(s) from an individual to be assessed at one or more polymorphic position(s) of the human IPF1 gene sequence selected from nucleotide position 4445 of SEQ ID NO:11 and/or SEQ ID NO:12, wherein the presence of the reference nucleotide at the one or more polymorphic position(s) indicates that the individual has a decreased likelihood of a favorable response to the administration of a pharmaceutically acceptable amount of a DPP-IV inhibitor as compared to an individual having the variant allele at said polymorphic position(s).

The invention further relates to a method for determining the likelihood that an individual will have a favorable response to the administration of a pharmaceutically acceptable amount of a DPP-IV inhibitor comprising the step of determining the nucleotide present within at least one or more nucleic acid sample(s) from an individual to be assessed at one or more polymorphic position(s) of the human IPF1 gene sequence selected from nucleotide position 4445 of SEQ ID NO:11 and/or SEQ ID NO:12, wherein the presence of the variant nucleotide at the one or more polymorphic position(s) indicates that the individual has an increased likelihood of a favorable response to the administration of a pharmaceutically acceptable amount of a DPP-IV inhibitor as compared to an individual having the reference allele at said polymorphic position(s).

### BRIEF DESCRIPTION OF THE FIGURES/DRAWINGS

FIGS. 1A-L show the polynucleotide sequence (SEQ ID NO:1) of SNP1 allele “G” of the human CYP3A5 sequence (referred to as isoform CYP3A5*1; gi|NM_{—}000777) comprising a predicted polynucleotide polymorphic locus located at nucleotide 7068 of SEQ ID NO:1. The polynucleotide sequence contains a sequence of 31790 nucleotides. The reference nucleotide at the polymorphic locus within the polynucleotide allele is a “G” and is denoted in bold and double underlining. Sequences corresponding to exon sequences of the CYP3A5 gene are represented by capital letters, whereas sequences corresponding to intron sequences are represented in lower capital letters.

FIGS. 2A-L show the polynucleotide sequence (SEQ ID NO:2) of SNP1 allele “A” of the human CYP3A5 sequence (referred to as isoform CYP3A5*3; Kuehl, P, et al., 2001, Nature Genetics, 27, pp. 383-391) comprising a predicted polynucleotide polymorphic locus located at nucleotide 7068 of SEQ ID NO:2. The polynucleotide sequence contains a sequence of 31790 nucleotides. The variant nucleotide at the polymorphic locus within the polynucleotide allele is an “A” and is denoted in bold and double underlining. Sequences corresponding to exon sequences of the CYP3A5 gene are represented by capital letters, whereas sequences corresponding to intron sequences are represented in lower capital letters.

FIG. 3 shows the statistical association between human CYP3A5 SNP1 alleles “G” (reference, isoform *1) and “A” (variant, isoform *3) with the likelihood of a favorable response to the administration of a pharmaceutically acceptable amount of a DPP-IV inhibitor. Results are shown in terms of fold incidence of each genotype residing in a patient that was part of non-responder and good responder DPP-IV inhibitor groups. As shown, “A” allele homozygous patients (“A/A”) at the SNP1 locus have a higher likelihood of a favorable response to the administration of a pharmaceutically acceptable amount of a DPP-IV inhibitor; heterozygous patients (“A/G”) at the SNP1 locus have a lower likelihood of a favorable response to the administration of a pharmaceutically acceptable amount of a DPP-IV inhibitor compared to homozygous “A/A” allele patients; while “G” allele homozygous patients (“G/G”) at the SNP1 locus have a significantly lower likelihood of a favorable response to the administration of a pharmaceutically acceptable amount of a DPP-IV inhibitor compared to homozygous “A” and heterozygous (“A/G”) allele patients.

FIGS. 4A-D show the polynucleotide sequence (SEQ ID NO:11) of SNP1 allele “C” of the human IPF-1 sequence (referred to as insulin promoter factor 1; gi|NM_{—}000209) comprising a predicted polynucleotide polymorphic locus located at nucleotide 4445 of SEQ ID NO:11. The polynucleotide sequence contains a sequence of 7218 nucleotides. The reference nucleotide at the polymorphic locus within the polynucleotide allele is a “C” and is denoted in bold and double underlining. Sequences corresponding to exon sequences of the IPF-1 gene are represented by capital letters, whereas sequences corresponding to intron sequences are represented in lower capital letters.

FIGS. 5A-D show the polynucleotide sequence (SEQ ID NO:12) of SNP1 allele “T” of the human IPF-1 sequence (referred to as insulin promoter factor 1) comprising a predicted polynucleotide polymorphic locus located at nucleotide 4445 of SEQ ID NO:12. The polynucleotide sequence contains a sequence of 7218 nucleotides. The variant nucleotide at the polymorphic locus within the polynucleotide allele is a “T” and is denoted in bold and double underlining. Sequences corresponding to exon sequences of the IPF-1 gene are represented by capital letters, whereas sequences corresponding to intron sequences are represented in lower capital letters.

FIG. 6 shows the statistical association between human IPF-1 SNP1 alleles “C” (reference) and “T” (variant) with the likelihood of a favorable response to the administration of a pharmaceutically acceptable amount of a DPP-IV inhibitor. Results are shown in terms of fold incidence of each genotype residing in a patient that was part of non-responder and good responder DPP-IV inhibitor groups. As shown, “T” allele homozygous patients (“T/T”) at the SNP1 locus have a higher likelihood of a favorable response to the administration of a pharmaceutically acceptable amount of a DPP-IV inhibitor; heterozygous patients (“C/T”) at the SNP1 locus also have a higher likelihood of a favorable response to the administration of a pharmaceutically acceptable amount of a DPP-IV inhibitor; while “C” allele homozygous patients (“C/C”) at the SNP1 locus have a lower likelihood of a favorable response to the administration of a pharmaceutically acceptable amount of a DPP-IV inhibitor compared to homozygous “T” and heterozygous (“C/T”) allele patients.

### DETAILED DESCRIPTION OF THE INVENTION

The present invention relates to a nucleic acid molecule comprising a single nucleotide polymorphism (SNP) at a specific location, referred to herein as the polymorphic locus, and complements thereof. The nucleic acid molecule, e.g., a gene, which includes the SNP has at least two alleles, referred to herein as the reference allele and the variant allele. The reference allele typically, but not always, corresponds to the nucleotide sequence of the native form of the nucleic acid molecule.

The present invention pertains to novel polynucleotides of the human CYP3A5 gene comprising at least one single nucleotide polymorphism (SNP) which has been shown to be associated with an increased likelihood of a favorable response to the administration of a pharmaceutically acceptable amount of a DPP-IV inhibitor. The CYP3A5 SNPs were identified by sequencing the CYP3A5 genomic sequence of a large number of individuals that were subjected to DPP-IV inhibitor therapy, and comparing the CYP3A5 sequences of those individuals who were non-responders to those individuals who were good responders of DPP-IV inhibition. Each of the novel CYP3A5 SNPs were located in the non-coding regions of the CYP3A5 gene and are thought to affect the splicing of the CYP3A5 gene in those patients containing one or more of these SNPs.

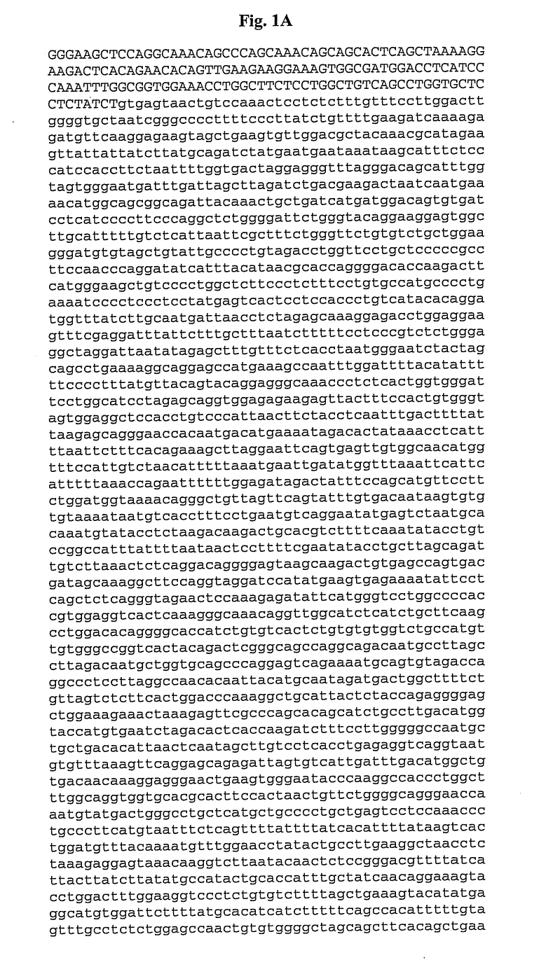

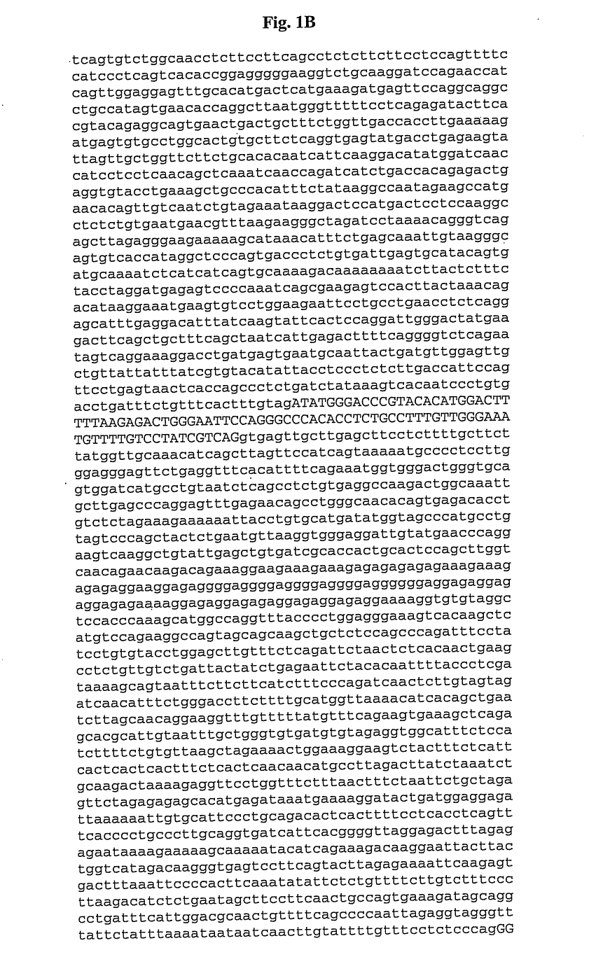

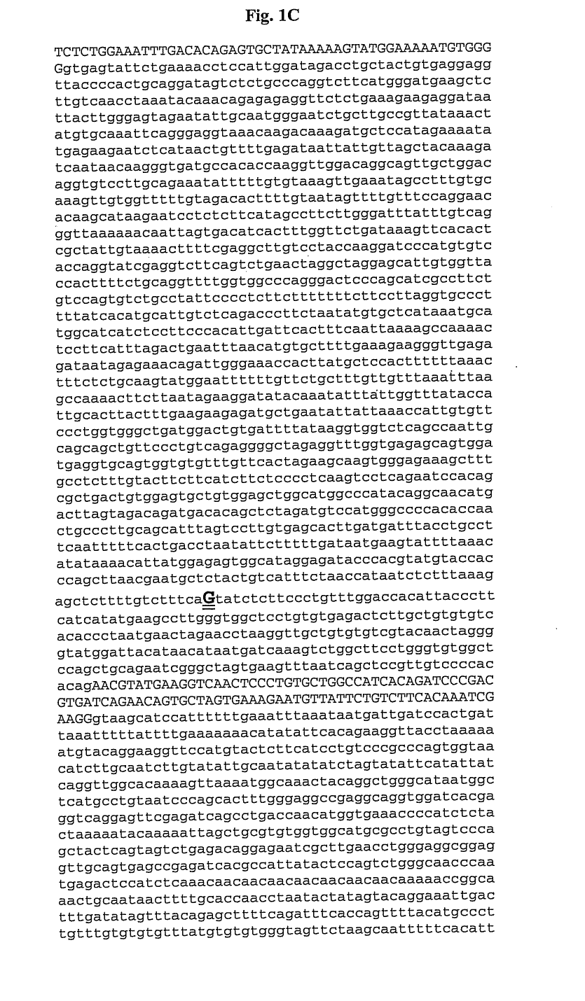

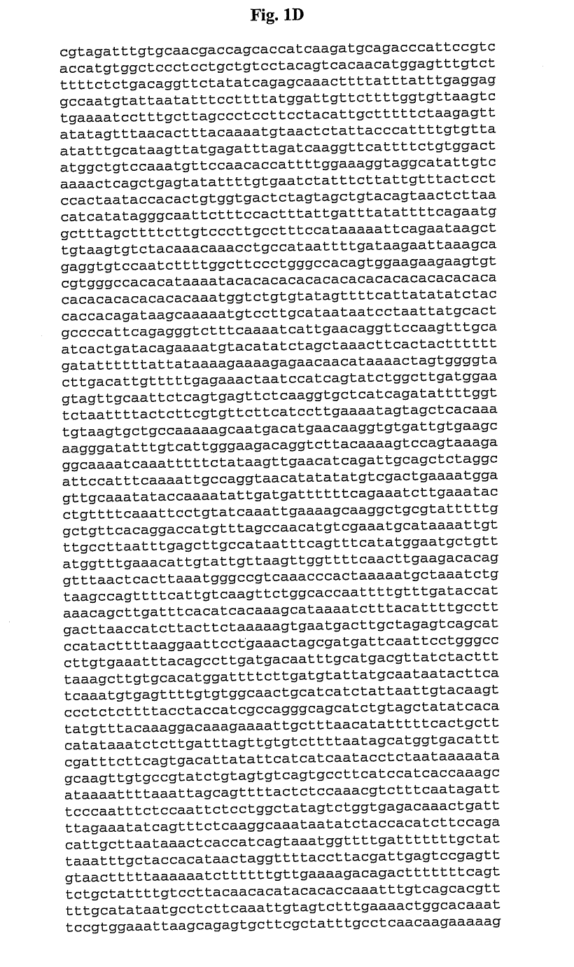

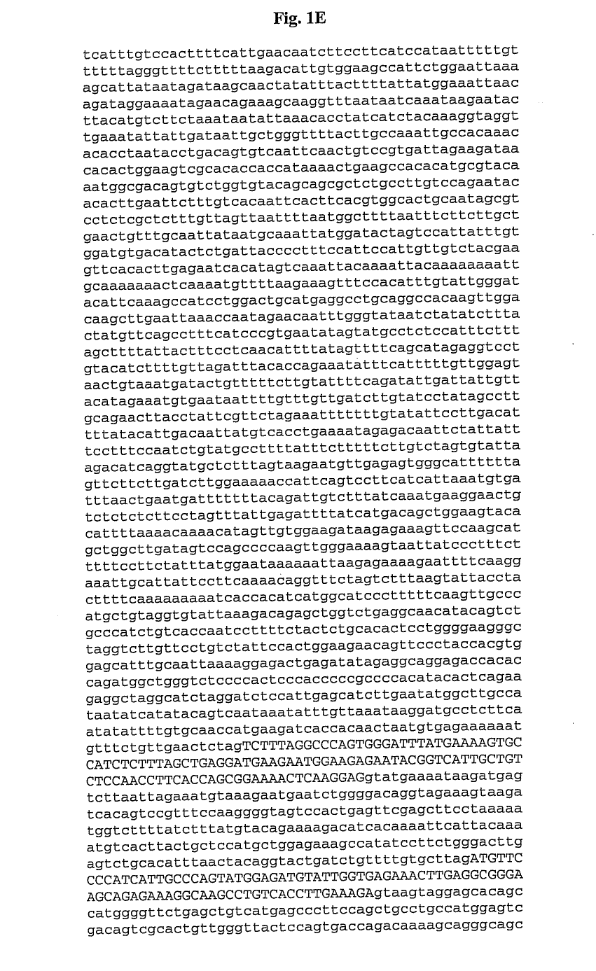

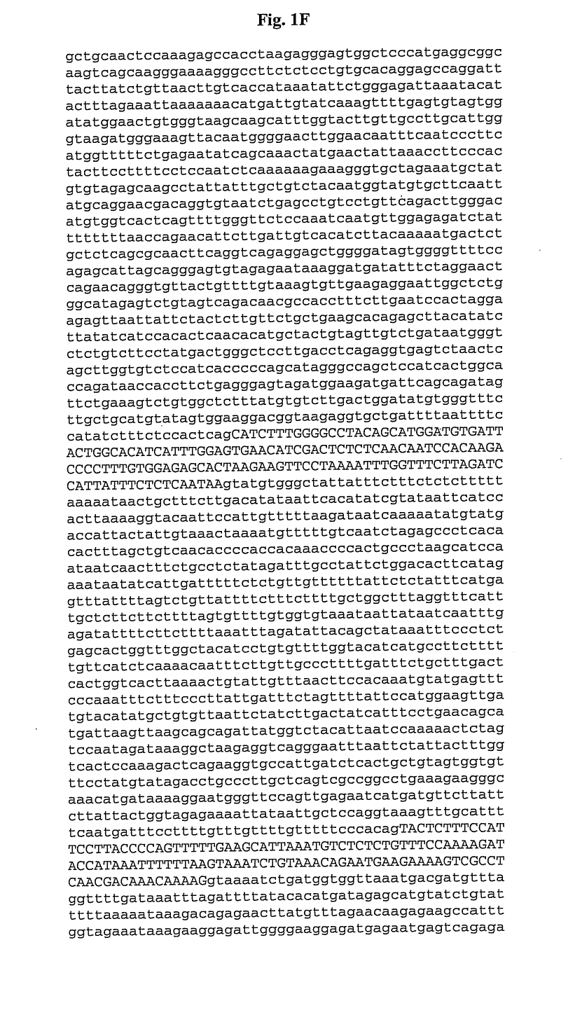

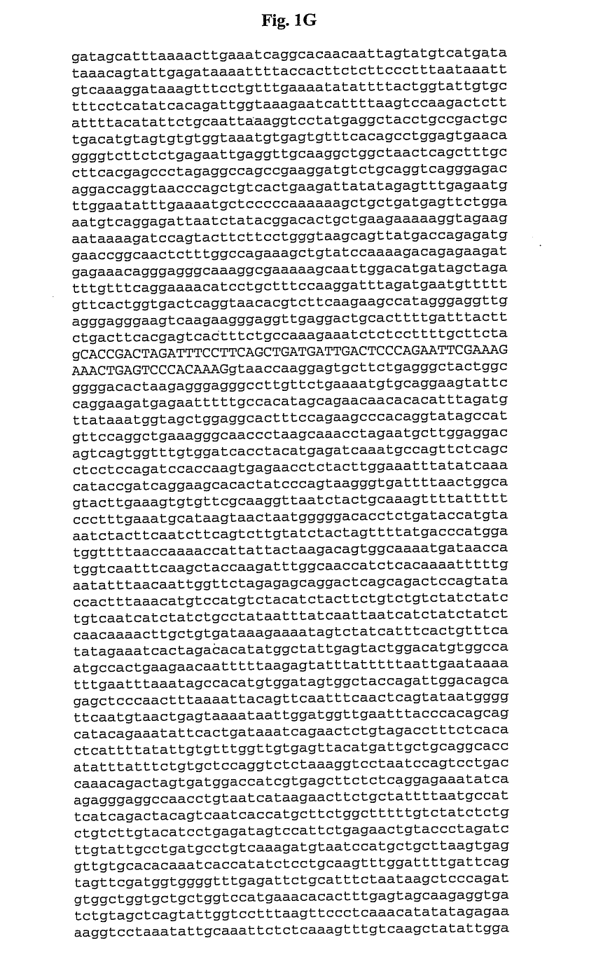

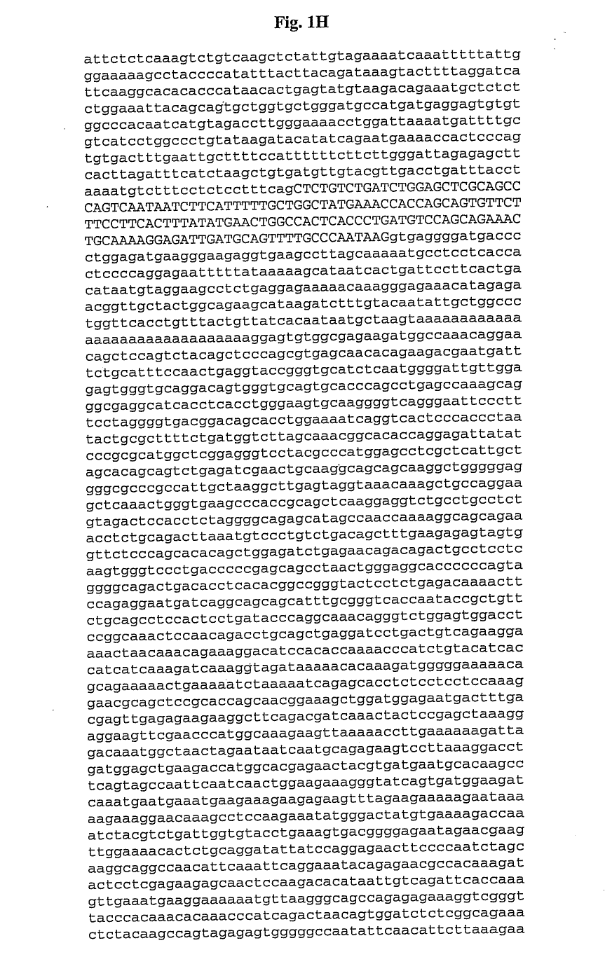

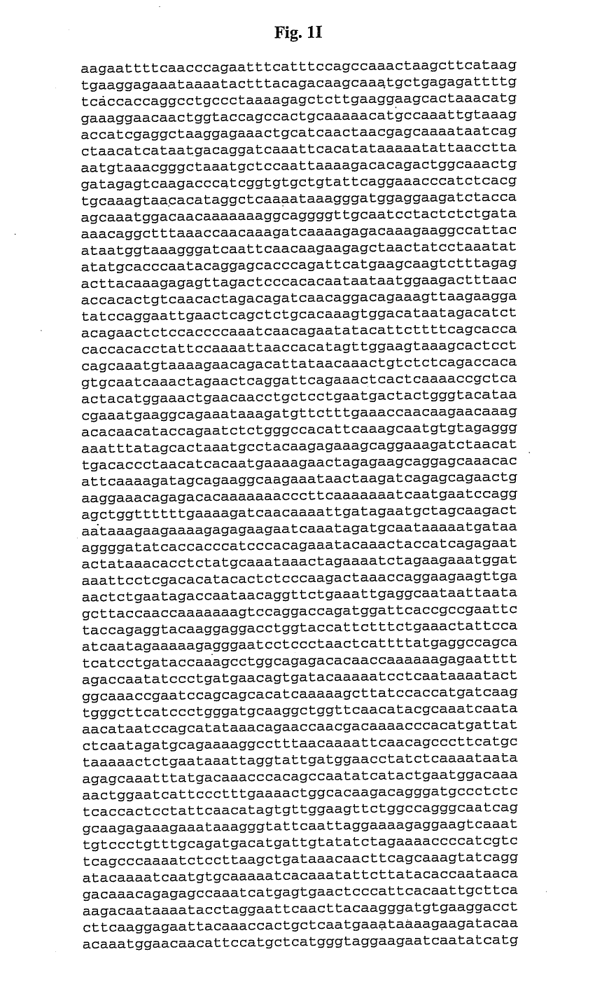

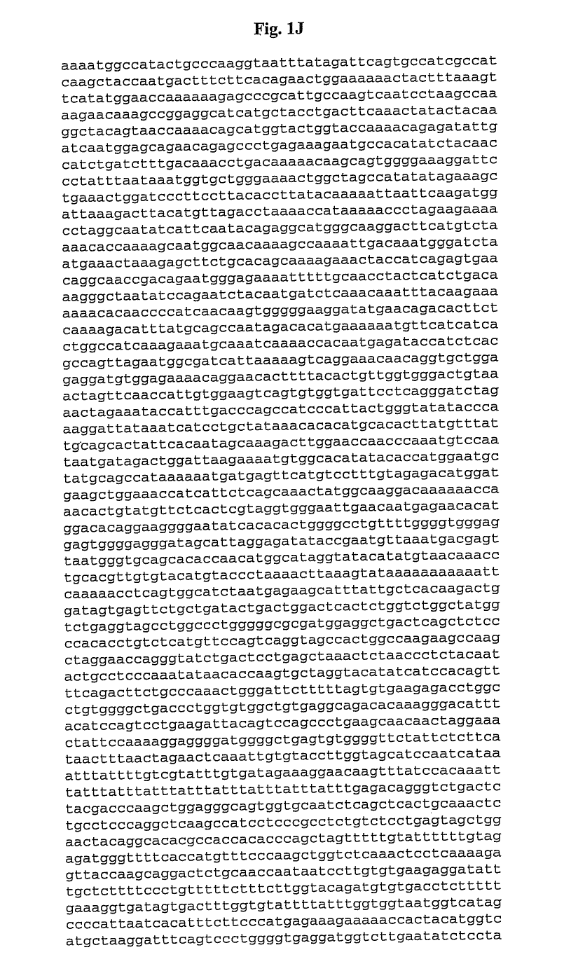

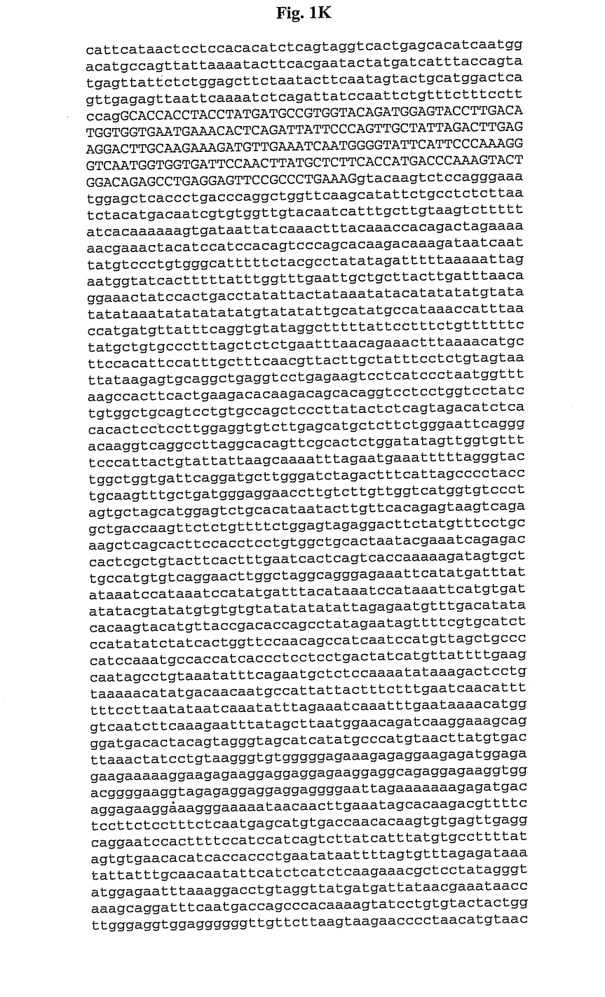

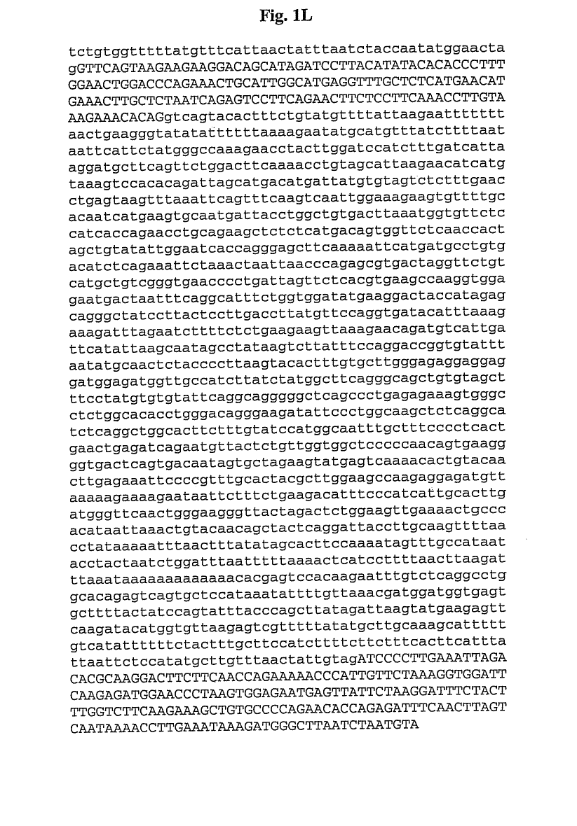

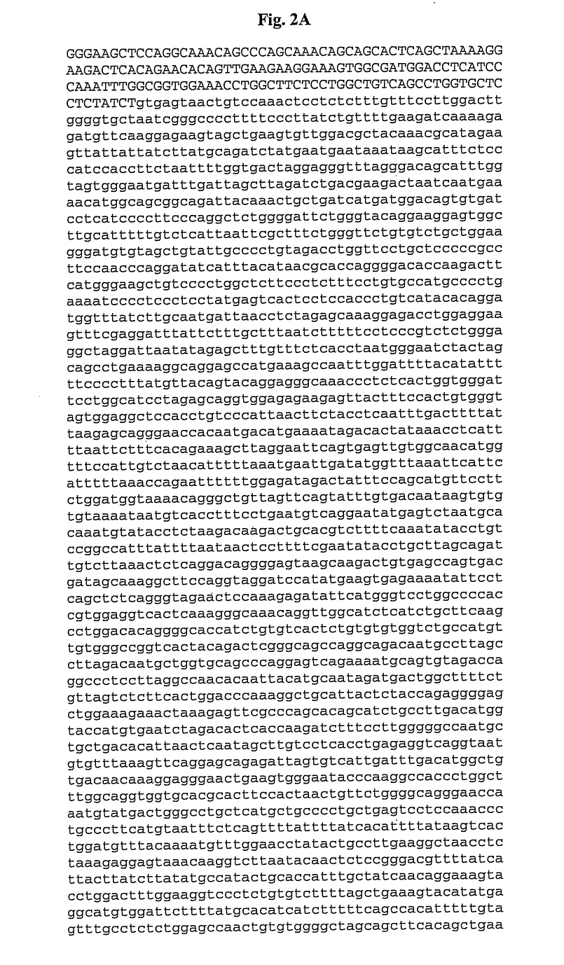

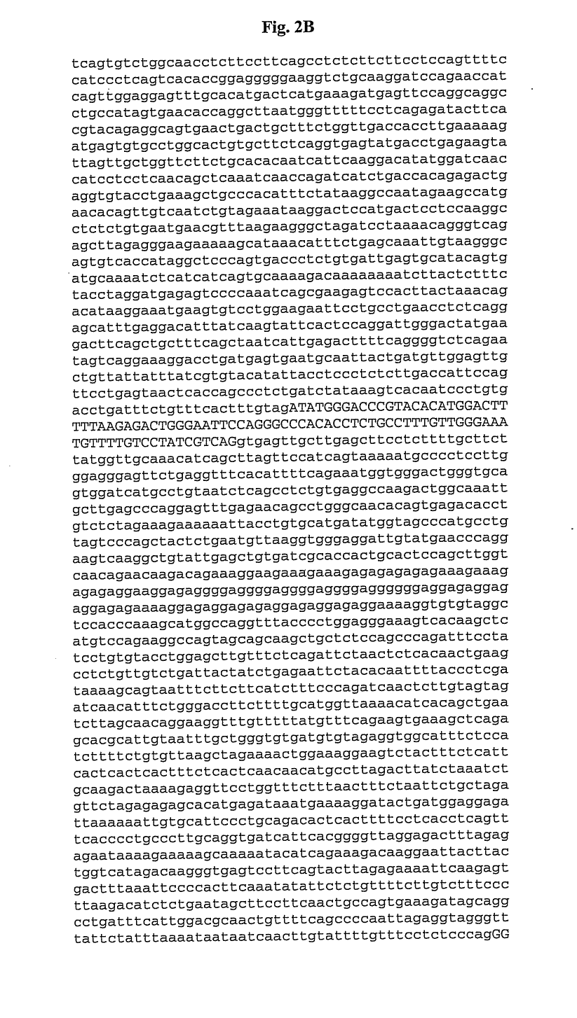

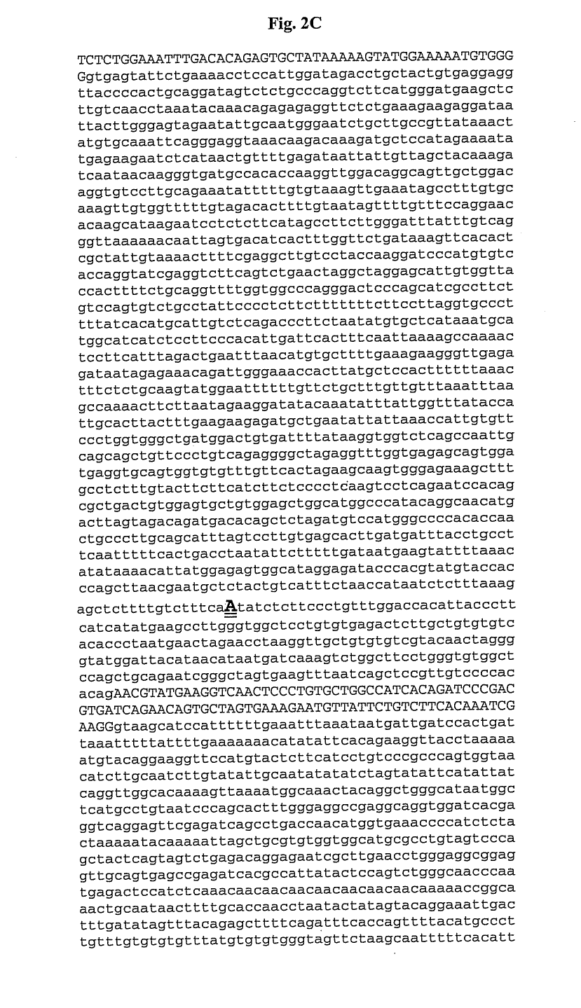

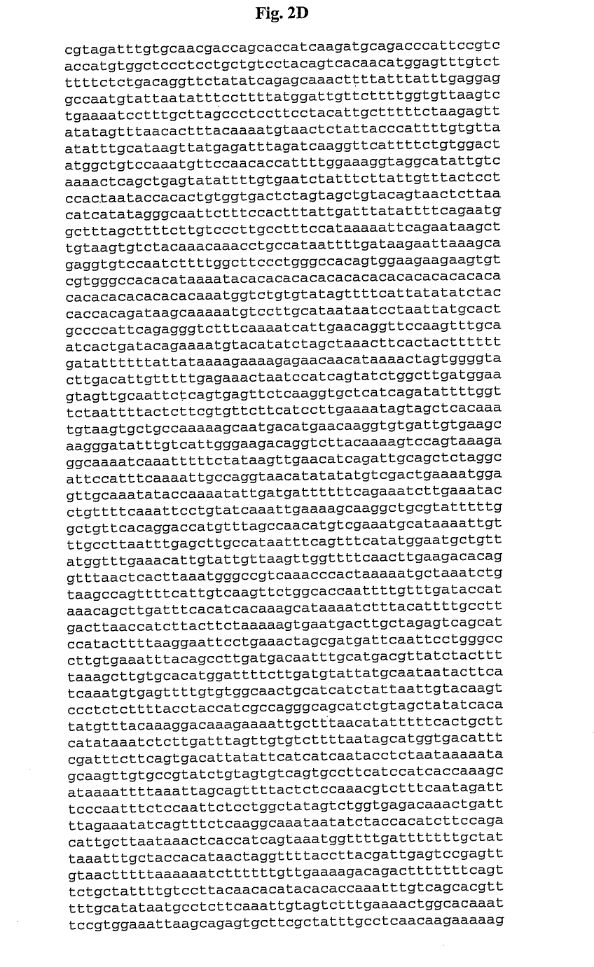

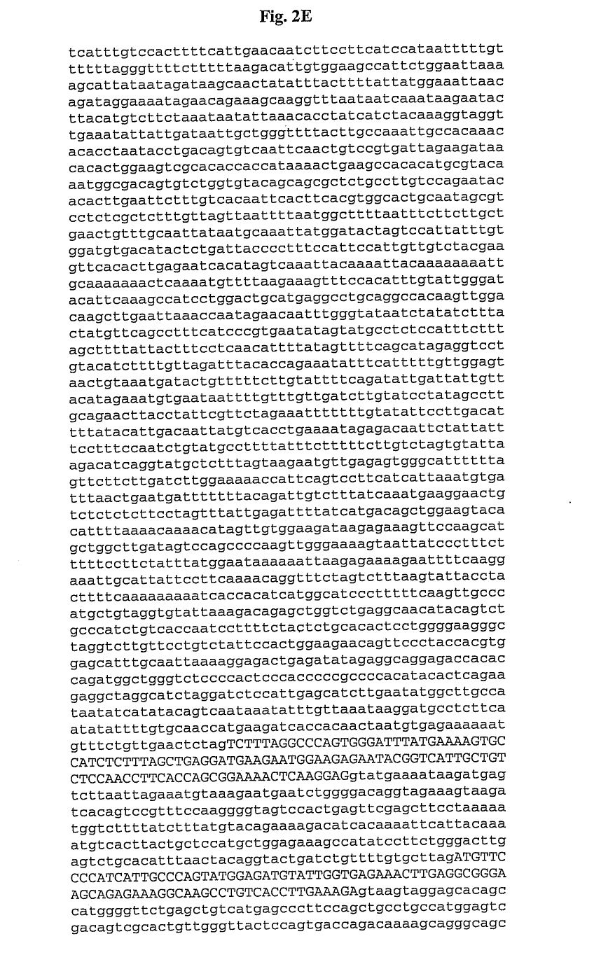

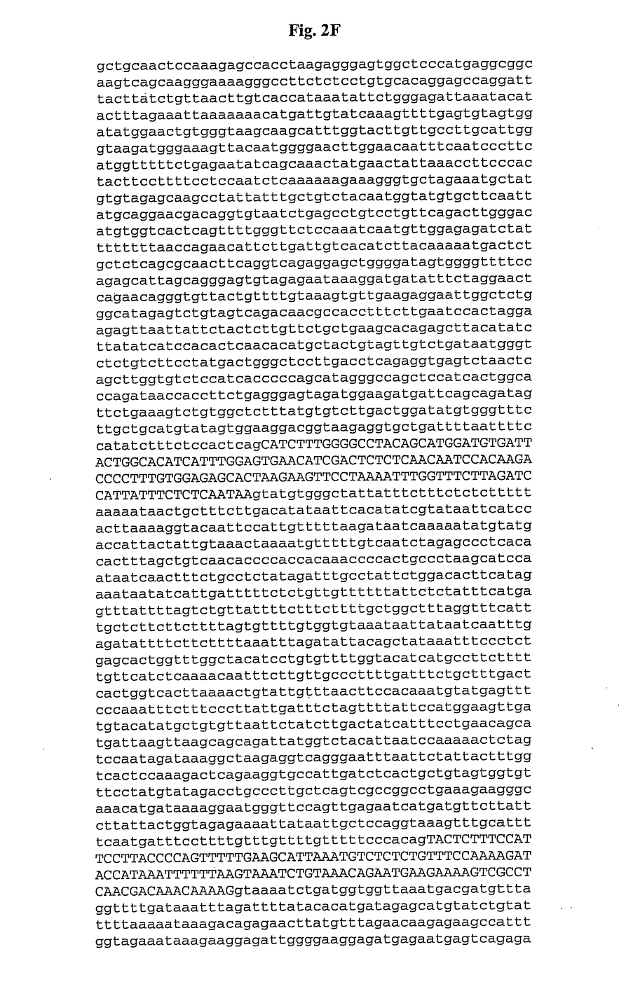

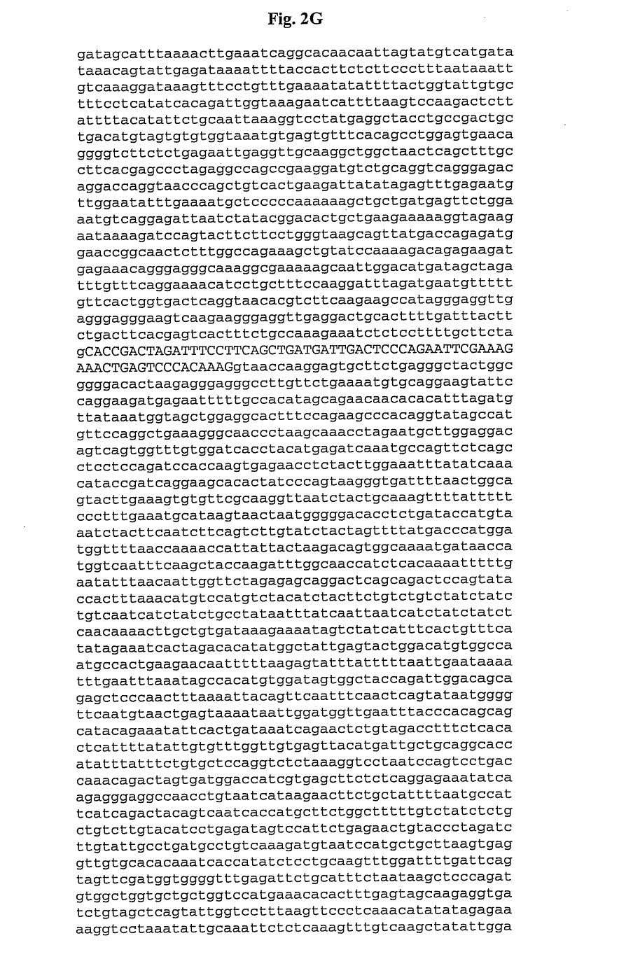

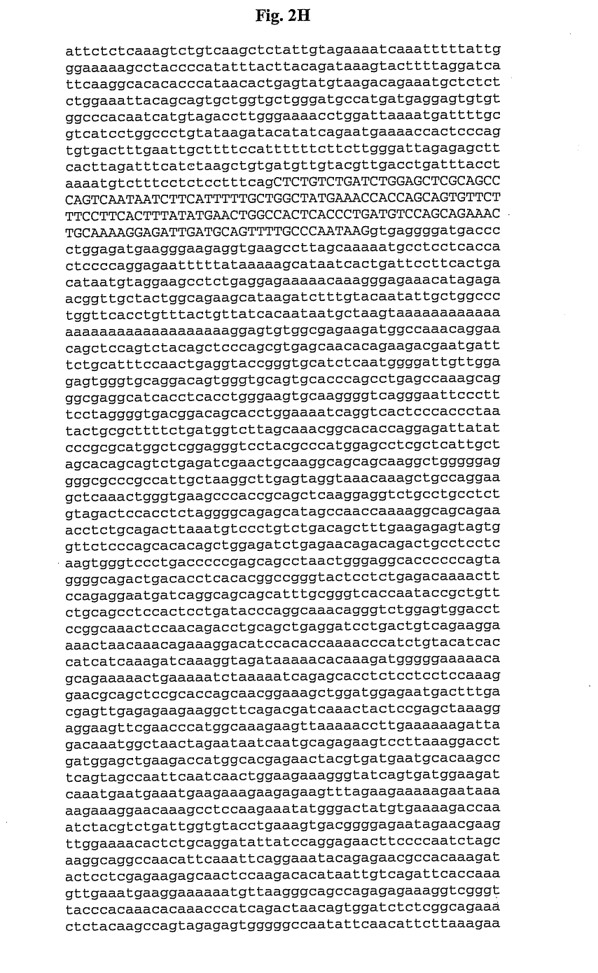

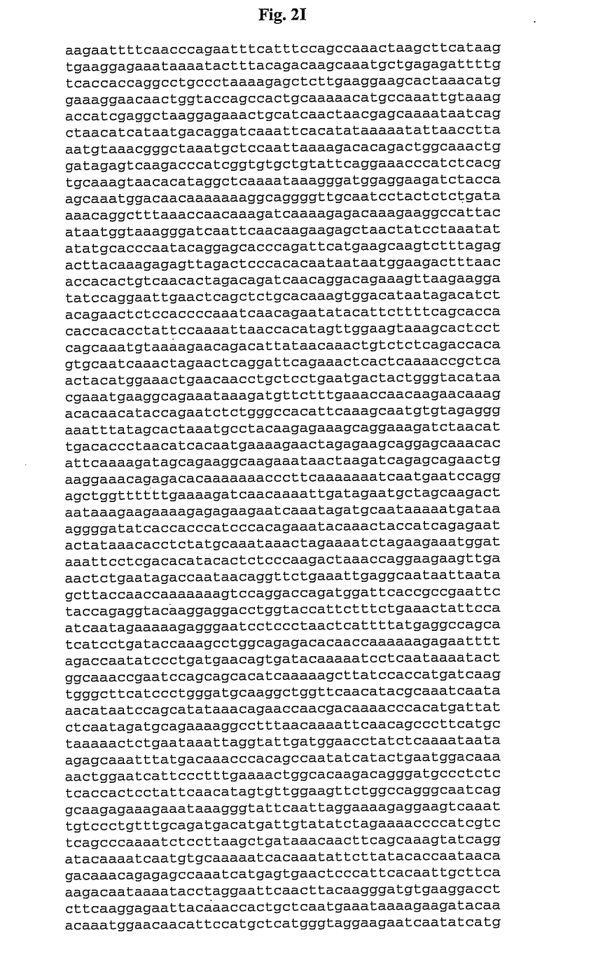

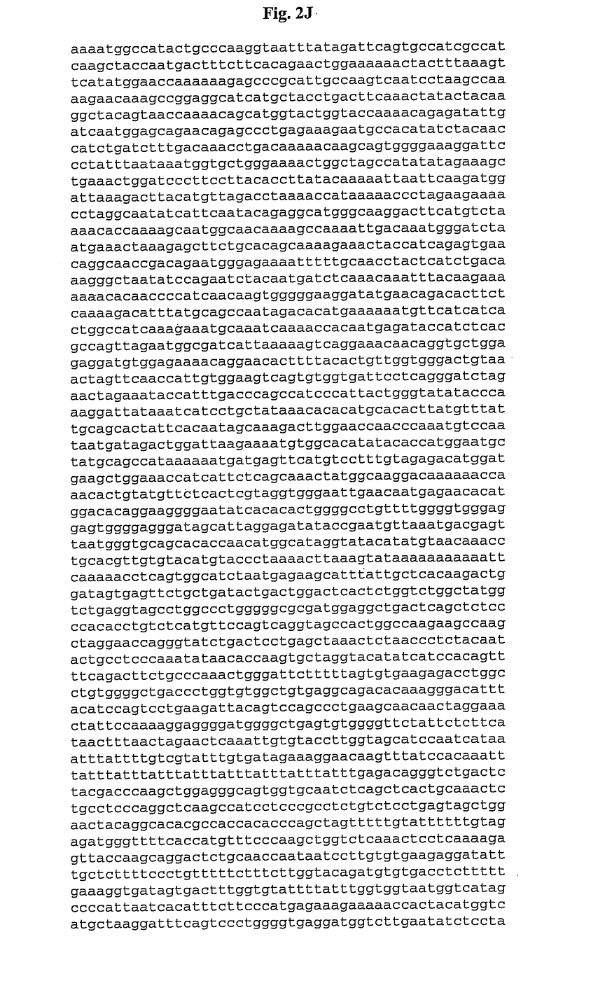

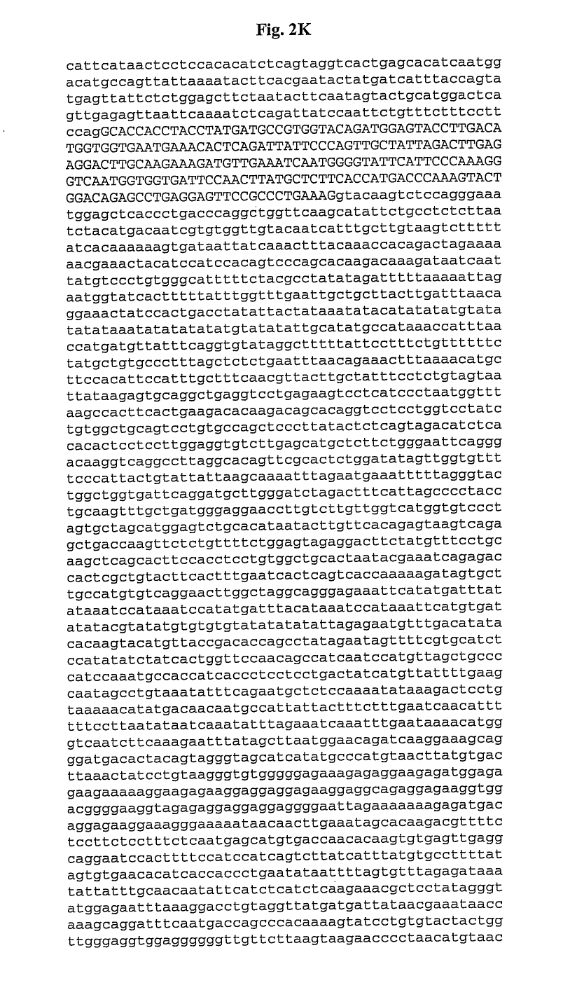

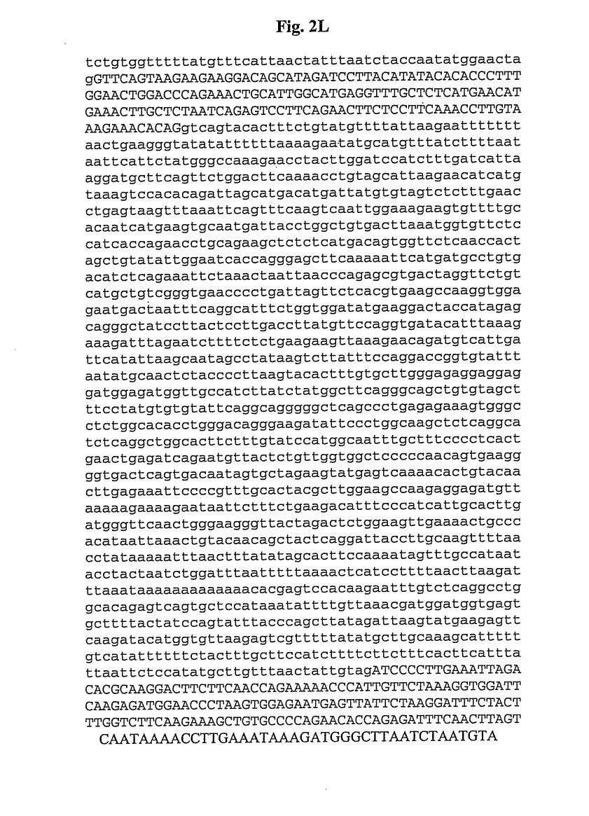

The present invention also relates to variant alleles of the described CYP3A5 gene and to complements of the variant alleles. The variant allele differs from the reference allele by one nucleotide at the polymorphic locus identified in the FIGS. 1A-L and/or FIGS. 2A-L.

The present invention also pertains to novel polynucleotides of the human IPF-1 gene comprising at least one single nucleotide polymorphism (SNP) which has been shown to be associated with an increased likelihood of a favorable response to the administration of a pharmaceutically acceptable amount of a DPP-IV inhibitor. The IPF-1 SNPs were identified by sequencing the IPF-1 genomic sequence of a large number of individuals that were subjected to DPP-IV inhibitor therapy, and comparing the IPF-1 sequences of those individuals who were non-responders to those individuals who were good responders of DPP-IV inhibition.

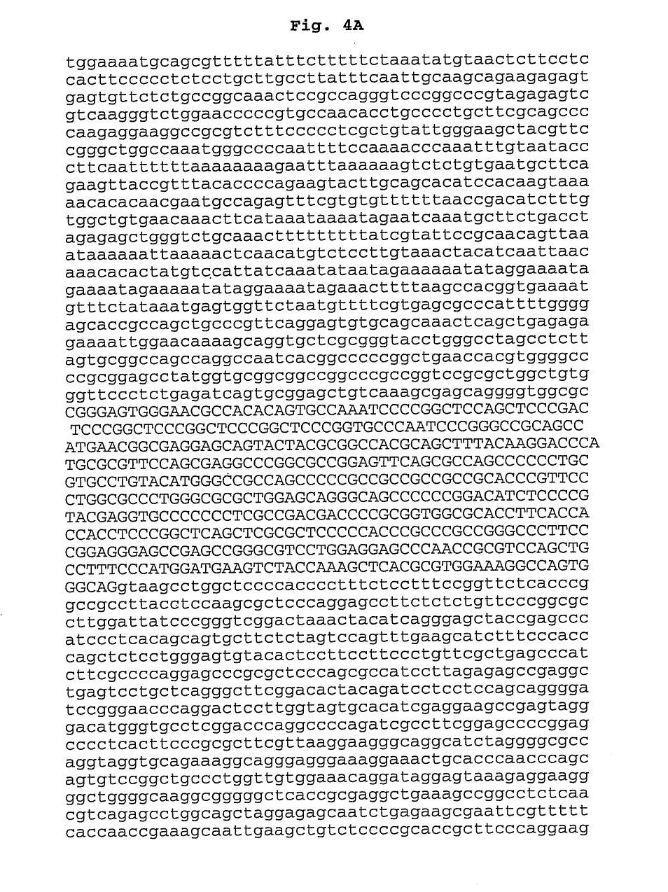

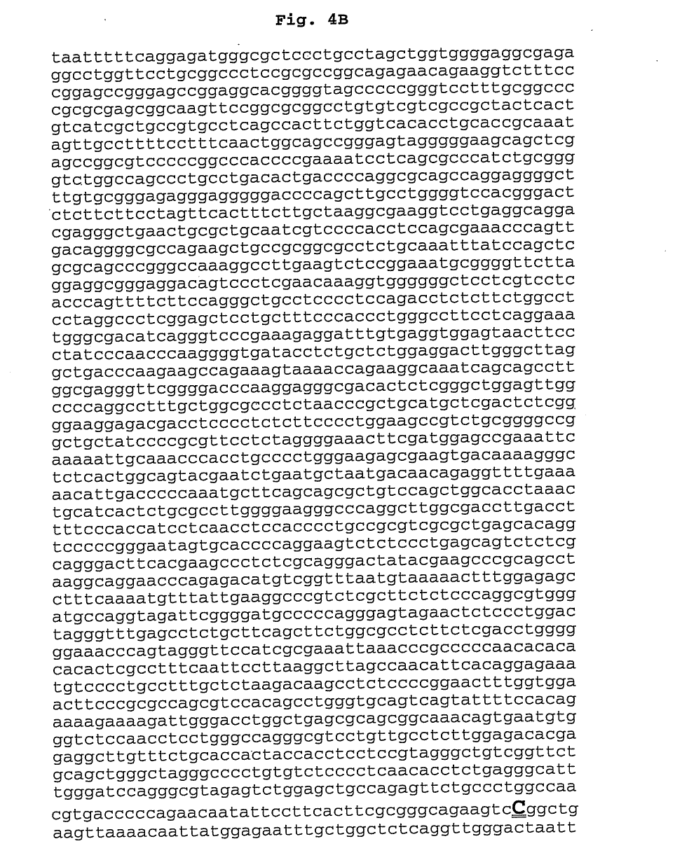

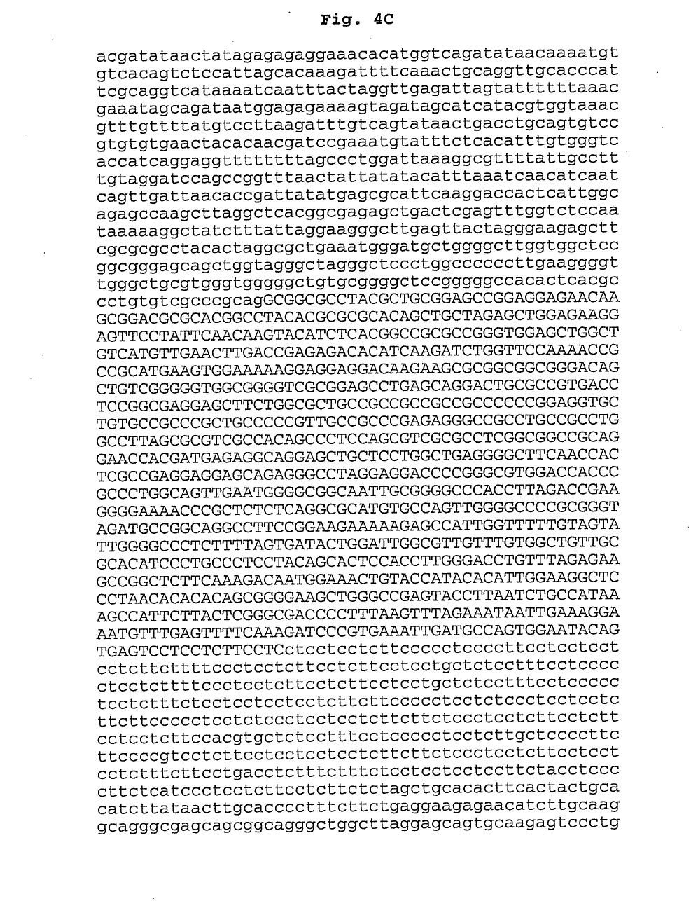

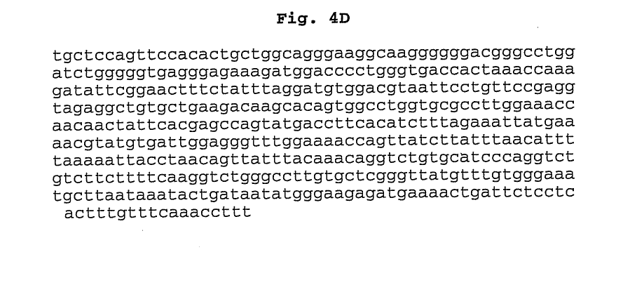

The present invention also relates to variant alleles of the described IPF-1 gene and to complements of the variant alleles. The variant allele differs from the reference allele by one nucleotide at the polymorphic locus identified in the FIGS. 4A-D and/or FIGS. 4A-D.

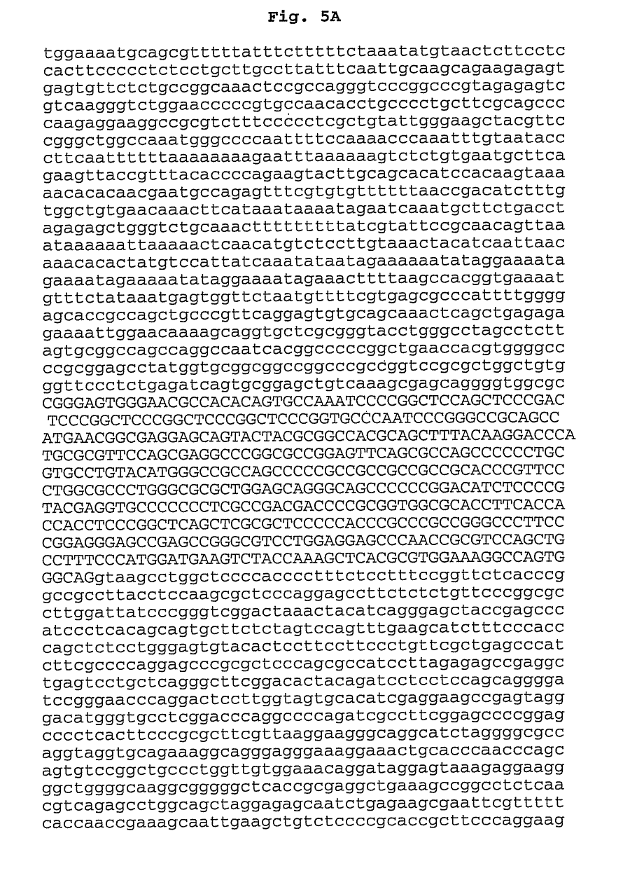

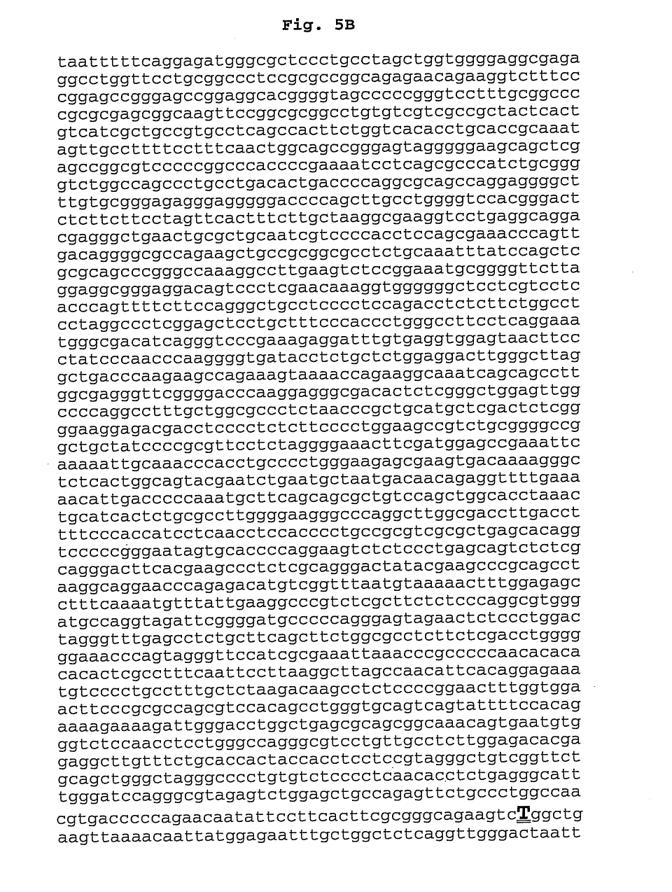

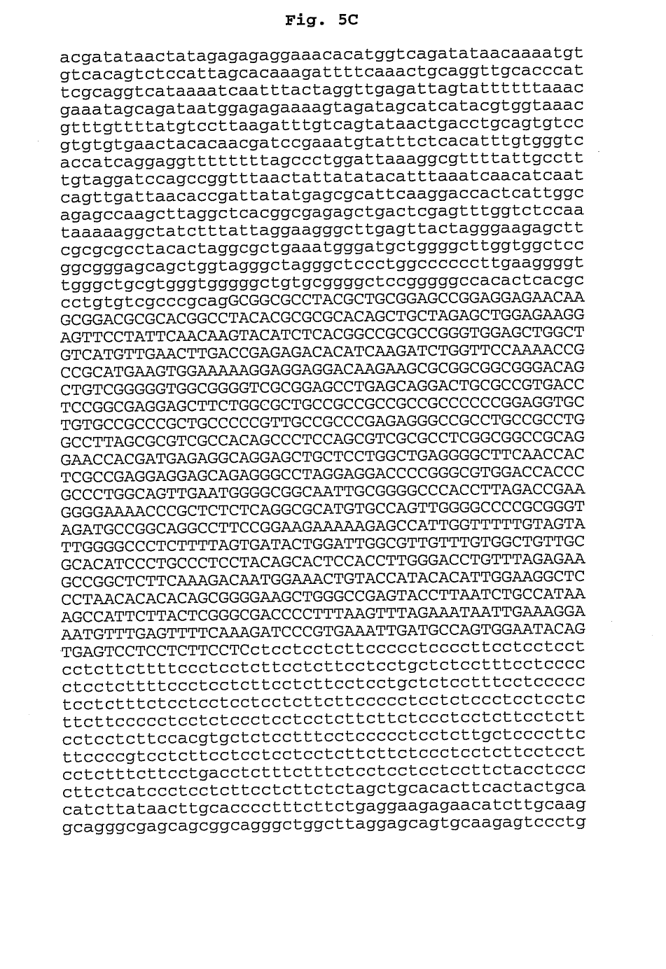

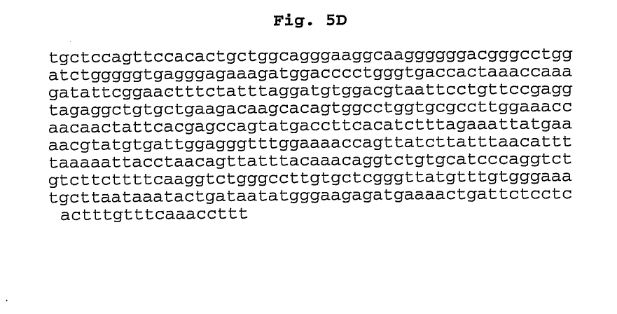

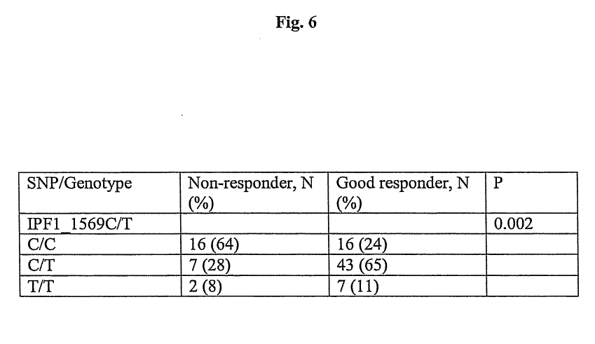

The invention further relates to fragments of the variant alleles and fragments of complements of the variant alleles which comprise the site of the SNP (e.g., polymorphic locus) and are at least five nucleotides in length. Fragments can be about 5 to about 100 nucleotides in length, for example, about 5-10 nucleotides, about 5-15 nucleotides, about 10-20 nucleotides, about 5-25 nucleotides, about 10-30 nucleotides, about 10-40 nucleotides, about 10-50 nucleotides or about 10-100 nucleotides. For example, a variant fragment or portion of a variant allele which is about 10 nucleotides in length comprises at least one single nucleotide polymorphism (the nucleotide which differs from the reference allele at the polymorphic locus) and nine additional nucleotides which flank the site in the variant allele. These additional nucleotides can be on one or both sides of the polymorphism. Examples of polymorphisms which are the subject of this invention are found in FIGS. 1A-L and/or FIGS. 2A-L for the CYP3A5 gene, and in FIGS. 4A-D and/or FIGS. 5A-D for the IPF-1 gene.

In one specific embodiment, the invention relates to the human CYP3A5 gene having a nucleotide sequence according to FIGS. 1A-L or FIGS. 2A-L (SEQ ID NO:1 or SEQ ID NO:2) comprising a single nucleotide polymorphism at a polymorphic locus found at nucleotide 7068 of SEQ ID NO:1 or SEQ ID NO:2. The reference nucleotide for the polymorphic locus at nucleotide 7068 is “G”. The variant nucleotide for the polymorphic locus at nucleotide 7068 is “A”. The nucleotide sequences of the present invention can be double- or single-stranded.

The invention further relates to a portion of the human CYP3A5 gene comprising one or more polymorphic loci selected from nucleotide 7068 of SEQ ID NO:1 and/or SEQ ID NO:2.

In another specific embodiment, the invention relates to the human IPF-1 gene having a nucleotide sequence according to FIGS. 4A-D or FIGS. 5A-D (SEQ ID NO:11 or SEQ ID NO:12) comprising a single nucleotide polymorphism at a polymorphic locus at nucleotide 4445 of SEQ ID NO:11 or SEQ ID NO:12. The reference nucleotide for the polymorphic locus at nucleotide 4445 is “C”. The variant nucleotide for the polymorphic locus at nucleotide 4445 is “T”. The nucleotide sequences of the present invention can be double- or single-stranded.

The invention further relates to a portion of the human IPF-1 gene comprising one or more polymorphic loci selected from nucleotide 4445 of SEQ ID NO:11 and/or SEQ ID NO:12.

The human CYP3A5 and IPF1 genes were chosen as candidate genes to investigate the association of one or more single nucleotide polymorphisms with an increased likelihood of a favorable response to the administration of a pharmaceutically acceptable amount of a DPP-IV inhibitor phenotype based upon the appreciation that these proteins are involved in the metabolism of DPP-IV inhibitors, in vivo. The single nucleotide polymorphisms described herein derived from the CYP3A5 or IPF-1 gene have been shown in the invention to be associated with an increased likelihood of a favorable response to the administration of a pharmaceutically acceptable amount of a DPP-IV inhibitor. Specifically, the reference single nucleotide polymorphisms of the human CYP3A5 or IPF-1 gene described herein have been demonstrated to statistically decrease the likelihood of a favorable response to the administration of a pharmaceutically acceptable amount of a DPP-IV inhibitor.

The invention further provides allele-specific oligonucleotides that hybridize to the human CYP3A5 or IPF-1 gene sequence, or fragments or complements thereof, comprising one or more single nucleotide polymorphisms and/or polymorphic locus. Such oligonucleotides are expected to hybridize to one polymorphic allele of the nucleic acid molecules described herein but not to the other polymorphic allele(s) of the sequence. Thus, such oligonucleotides can be used to determine the presence or absence of particular alleles of the polymorphic sequences described herein and to distinguish between reference and variant allele for each form. These oligonucleotides can be probes or primers, such as the primers provided herein.

The described polynucleotides and oligonucleotides of the invention, as well as the corresponding methods described herein, can be used to analyze a nucleic acid from an individual to identify the presence or absence of a particular nucleotide at a given polymorphic locus and to distinguish between the reference and variant allele at each locus. In one embodiment, the method of analyzing the nucleic acid comprises determining which base is present at any one of the polymorphic loci shown in FIGS. 1A-L and/or FIGS. 2A-L for the CYP3A5 gene (SEQ ID NOs:1 or 2), and in FIGS. 4A-D and/or FIGS. 5A-D for the IPF1 gene (SEQ ID NOs:11 or 12), or elsewhere herein. Optionally, a set of bases occupying a set of the polymorphic loci shown in FIGS. 1A-L and/or FIGS. 2A-L for the CYP3A5 gene (SEQ ID NOs:1 or 2), and in FIGS. 4A-D and/or FIGS. 5A-D for the IPF1 gene (SEQ ID NOs:11 or 12) is determined. This type of analysis can also be performed on a number of individuals, who are additionally tested (previously, concurrently or subsequently) for the presence of an increased likelihood of a favorable response to the administration of a pharmaceutically acceptable amount of a DPP-IV inhibitor phenotype in the presence or absence of a DPP-IV protease inhibitor. The presence or absence of an increased likelihood of a favorable response to the administration of a pharmaceutically acceptable amount of a DPP-IV inhibitor phenotype is then correlated with a base or set of bases present at the polymorphic locus or loci in the patient and/or sample tested.

Thus, the invention further provides a method of determining the likelihood (e.g., increased, decreased, or no likelihood) of a favorable response to the administration of a pharmaceutically acceptable amount of a DPP-IV inhibitor phenotype associated with a particular genotype in the presence or absence of a DPP-IV inhibitor. The method comprises obtaining a nucleic acid sample from an individual and determining the identity of one or more bases (nucleotides) at one or more polymorphic loci of the nucleic acid molecules described herein, wherein the presence of a particular base is correlated with the incidence of an increased likelihood of a favorable response to the administration of a pharmaceutically acceptable amount of a DPP-IV inhibitor phenotype in the presence of a DPP-IV inhibitor, thereby determining the likelihood of a favorable response to the administration of a pharmaceutically acceptable amount of a DPP-IV inhibitor in the individual or sample. The correlation between a particular polymorphic form of a gene and a phenotype can thus be used in methods of diagnosis of that phenotype, as well as in the development of various treatments for the phenotype.

### DEFINITIONS

An “oligonucleotide” can be DNA or RNA, and single- or double-stranded. An oligonucleotide can be used, for example, as either a “primer” or a “probe”. Oligonucleotides can be naturally occurring or synthetic, but are typically prepared by synthetic means. An oligonucleotide primer, for example, can be designed to hybridize to the complementary sequence of either the sense or antisense strand of a specific target sequence, and can be used alone or as a pair, such as in DNA amplification reactions, and may or may not comprise one or more polymorphic loci of the present invention. An oligonucleotide probe can also be designed to hybridize to the complementary sequence of either the sense or antisense strand of a specific target sequence, and can be used alone or as a pair, such as in DNA amplification reactions, but necessarily will comprise one or more polymorphic loci of the present invention. Preferred oligonucleotides of the invention include fragments of DNA, and their complements thereof, of the human CYP3A5 or IPF-1 gene, and can comprise one or more of the polymorphic loci shown or described in FIGS. 1A-L and/or FIGS. 2A-L for the CYP3A5 gene (SEQ ID NOs:1 and 2), and in FIGS. 4A-D and/or FIGS. 5A-D for the IPF-1 gene (SEQ ID NOs:11 and 12) or as described elsewhere herein. The fragments can be about 10 to about 250 nucleotides and, in specific embodiments, are about 5 to about 100 nucleotides in length, including, for example, about 5 to about 10nucleotides, about 5 to about 15 nucleotides, about 10 to about 20 nucleotides, about 15 to about 25 nucleotides, about 10 to about 30 nucleotides, about 10 to about 40 nucleotides, about 10 to about 50 nucleotides, and about 50 to about 100 nucleotides in length. For example, the fragment can be 40 nucleotides in length. The polymorphic locus can occur within any nucleotide position of the fragment, including at either terminal position or any internal position, including directly in the middle of the fragment. The fragments can be from any of the allelic forms of DNA shown or described herein.

As used herein, the terms “nucleotide”, “base” and “nucleic acid” are intended to be equivalent. The terms “nucleotide sequence”, “nucleic acid sequence”, “nucleic acid molecule” and “nucleic acid segment” are intended to be equivalent.

Hybridization probes are oligonucleotides that bind in a base-specific manner to a complementary strand of nucleic acid and are designed to identify the allele at one or more polymorphic loci, for example, within the CYP3A5 or IPF1 gene of the present invention. Such probes include peptide nucleic acids, as described in Nielsen et al., 1991, Science 254, 1497-1500. Probes can be any length suitable for specific hybridization to the target nucleic acid sequence. The most appropriate length of the probe may vary depending upon the hybridization method in which it is being used; for example, particular lengths may be more appropriate for use in microfabricated arrays, while other lengths may be more suitable for use in classical hybridization methods. Such hybridization optimizations are known to the skilled artisan. Suitable probes can range from about 4 nucleotides to about 40 nucleotides, including about 12 nucleotides to about 25 nucleotides in length. For example, probes and primers can be about 4, 6, 8, 10, 12, 14, 16, 18, 20, 22, 24, 25, 26, 30, 35, or about 40 nucleotides in length. The probe preferably comprises at least one polymorphic locus occupied by any of the possible variant nucleotides. For comparison purposes, the present invention also encompasses probes that comprise the reference nucleotide at least one polymorphic locus. The nucleotide sequence can correspond to the coding sequence of the allele or to the complement of the coding sequence of the allele, where applicable.

Probe hybridizations are usually performed under stringent conditions, for example, at a salt concentration of no more than 1 M and a temperature of at least 25° C. For example, conditions of 5×SSPE and a temperature of 25-30° C., or equivalent conditions, are suitable for allele-specific probe hybridizations. Equivalent conditions can be determined by varying one or more of the parameters given as an example, as known in the art, while maintaining a similar degree of identity or similarity between the target nucleotide sequence and the primer or probe used.

As used herein, the term “primer” refers to a single-stranded oligonucleotide which acts as a point of initiation of template-directed DNA synthesis under appropriate conditions. Such DNA synthesis reactions can be carried out in the traditional method of including all four different nucleoside triphosphates (e.g., in the form of phosphoramidates, for example) corresponding to adenine, guanine, cytosine and thymine or uracil nucleotides, and an agent for polymerization, such as DNA or RNA polymerase or reverse transcriptase in an appropriate buffer and at a suitable temperature. Alternatively, such a DNA synthesis reaction may utilize only a single nucleoside (e.g., for single base-pair extension assays). The appropriate length of a primer depends on the intended use of the primer, but typically ranges from about 10 to about 30 nucleotides. Short primer molecules generally require cooler temperatures to form sufficiently stable hybrid complexes with the template. A primer need not reflect the exact sequence of the template, but must be sufficiently complementary to hybridize with a template. The term “primer site” refers to the area of the target DNA to which a primer hybridizes. The term primer pair refers to a set of primers including a 5′ (upstream) primer that hybridizes with the 5′ end of the DNA sequence to be amplified and a 3′ (downstream) primer that hybridizes with the complement of the 3′ end of the sequence to be amplified.

As used herein, “linkage” describes the tendency of genes, alleles, loci or genetic markers to be inherited together as a result of their location on the same chromosome. It can be measured by percent recombination between the two genes, alleles, loci or genetic markers.

As used herein, “polymorphism” refers to the occurrence of two or more genetically determined alternative sequences or alleles in a population. A “polymorphic locus” is a marker or site at which divergence from a reference allele occurs. The phrase “polymorphic loci” is meant to refer to two or more markers or sites at which divergence from two or more reference alleles occurs. Preferred markers have at least two alleles, each occurring at frequency of greater than 1%, and more preferably at a frequency greater than 10%-20% of a selected population. A polymorphic locus can be as small as one base pair. Polymorphic loci include, for example, restriction fragment length polymorphisms, variable number of tandem repeats (VNTR's), hypervariable regions, minisatellites, dinucleotide repeats, trinucleotide repeats, tetranucleotide repeats, simple sequence repeats, and insertion elements such as Alu. Typically, the first identified allelic form is arbitrarily designated as the “reference form” or “reference allele” and other allelic forms are designated as alternative forms or “variant alleles”. Diploid organisms may be homozygous or heterozygous for allelic forms. A diallelic or biallelic polymorphism has two forms. A triallelic polymorphism has three forms.

As used herein, the term “genotype” is meant to encompass the particular allele present at a polymorphic locus of a DNA sample, a gene, and/or chromosome.

As used herein, the term “haplotype” is meant to encompass the combination of genotypes across two or more polymorphic loci of a DNA sample, a gene, and/or chromosome, wherein the genotypes are closely linked, may be inherited together as a unit, and may be in linkage disequilibrium relative to other haplotypes and/or genotypes of other DNA samples, genes, and/or chromosomes.

As used herein, the term “linkage disequilibrium” refers to a measure of the degree of association between two alleles in a population. For example, when alleles at two distinctive loci occur in a sample more frequently than expected given the known allele frequencies and recombination fraction between the two loci, the two alleles may be described as being in “linkage disequilibrium”.

As used herein, the terms “genotype assay” and “genotype determination”, and the phrase “to genotype” or the verb usage of the term “genotype” are intended to be equivalent and refer to assays designed to identify the allele or alleles at a particular polymorphic locus or loci in a DNA sample, a gene, and/or chromosome. Such assays can employ, for example, single base extension reactions, DNA amplification reactions that amplify across one or more polymorphic loci, or may be as simple as sequencing across one or more polymorphic loci. A number of methods are known in the art for genotyping, with many of these assays being described herein or referred to herein.

The invention described herein pertains to the resequencing of the human CYP3A5 and/or IPF-1 gene in a large number of individuals to identify polymorphisms which may predispose individuals to an increased likelihood of a favorable response to an administered DPP-IV inhibitor. For example, polymorphisms in the CYP3A5 and/or IPF-1 gene described herein are associated with an increased likelihood of a favorable response to an administered DPP-IV inhibitor and are useful for predicting the likelihood that an individual will have such a response upon the administration of a DPP-IV inhibitor.

By altering amino acid sequence, SNPs may alter the function of the encoded proteins. The discovery of the SNP facilitates biochemical analysis of the variants and the development of assays to characterize the variants and to screen for pharmaceutical compounds that would interact directly with one or another form of the protein. SNPs (including silent SNPs) can also alter the regulation of the gene at the transcriptional or post-transcriptional level. SNPs (including silent SNPs) also enable the development of specific DNA, RNA, or protein-based diagnostics that detect the presence or absence of the polymorphism in particular conditions.

The phrase “DPP-IV inhibitor” is meant to encompass compounds, including, but not limited to, saxagliptin; 2-[4-{{2-(2S,5R)-2-cyano-5-ethynyl-1-pyrrolidinyl]-2-oxoethyl]amino]-4-methyl-1-piperidinyl]-4-pyridinecarboxylic acid (ABT-279); 7-But-2-ynyl-9-(6-methoxy-pyridin-3-yl)-6-piperazin-1-yl-7,9-dihydro-purin-8-one; E3024, 3-but-2-ynyl-5-methyl-2-piperazin-1-yl-3,5-dihydro-4H-imidazo[4,5-d]pyridazin-4-one tosylate; Sitagliptin; cis-2,5-dicyanopyrrolidine; 2-[3-[2-[(2S)-2-Cyano-1-pyrrolidinyl]-2-oxoethylamino]-3-methyl-1-oxobutyl]-1,2,3,4-tetrahydroisoquinoline; 2-Cyano-4-fluoro-1-thiovalylpyrrolidine analogues; KR-62436, 6-{2-[2-(5-cyano-4,5-dihydropyrazol-1-yl)-2-oxoethylamino]ethylamino]nicotinonitrile; Glutamic acid analogues; Vildagliptin ((2S)-{[(3-hydroxyadamantan-1-yl)amino]acetyl}-pyrrolidine-2-carbonitrile; 1-((S)-gamma-substituted prolyl)-(S)-2-cyanopyrrolidine; (2R)-4-oxo-443-(trifluoromethyl)-5,6-dihydro[1,2,4]triazolo[4,3-a]pyrazin-7(8H)-yl]-1-(2,4,5-trifluorophenyl)butan-2-amine; aminomethylpyrimidine; Gamma-amino-substituted analogues of 1-[(S)-2,4-diaminobutanoyl]piperidine; 1-[[(3-hydroxy-1-adamantyl)amino]acetyl]-2-cyano-(S)-pyrrolidine; NVP-DPP728 (1-[[[2-[(5-cyanopyridin-2-yl)amino]ethyl]amino]acetyl]-2-cyano-(S)-pyrrolidine); 1-[2-[(5-Cyanopyridin-2-yl)amino]ethylamino]acetyl-2-(S)-pyrrolidinecarbonitrile; FE 999011; in addition to any other DPP-IV inhibitor known in the art, as well as any salt, formulation and/or combination of the same.

“Saxagliptin” refers to the compound with the chemical name (1S,3S,5S)-2-[(2S)-2-amino-2-(3-hydroxytricyclo [3.3.1.1^{3,7}]dec-1-yl)-1-oxoethyl]-2-azabicyclo [3.1.0]hexane-3-carbonitrile or the alternative chemical name (1S,3S,5S)-2-[(2S)-2-amino-2-(3-hydroxy-1-adamantyl)-1-oxoethyl]-2-azabicyclo[3.1.0]hexane-3-carbonitrile having the formula provided as (I) below, as well as any pharmaceutically acceptable salt of this compound, any solvate or hydrate of the compound, any solvate of a pharmaceutically acceptable salt of the compound, and any crystal form of the compound or of a pharmaceutically acceptable salt of the compound, solvate of the compound, or solvate of a pharmaceutically acceptable salt of the compound. Saxagliptin is disclosed in U.S. Pat. No. 6,395,767 (exemplified in Example 60), which is incorporated in its entirety herein.

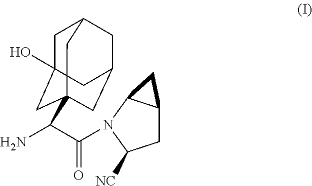

A single nucleotide polymorphism occurs at a polymorphic locus occupied by a single nucleotide, which is the site of variation between allelic sequences. The site is usually preceded by and followed by highly conserved sequences of the allele (e.g., sequences that vary in less than 1/100 or 1/1000 members of the populations).

A single nucleotide polymorphism usually arises due to substitution of one nucleotide for another at the polymorphic locus. A transition is the replacement of one purine by another purine or one pyrimidine by another pyrimidine. A transversion is the replacement of a purine by a pyrimidine or vice versa. Single nucleotide polymorphisms can also arise from a deletion of a nucleotide or an insertion of a nucleotide relative to a reference allele. Typically the polymorphic locus is occupied by a base other than the reference base. For example, where the reference allele contains the base “C” at the polymorphic site, the altered allele can contain a “T”, “G” or “A” at the polymorphic locus.

For the purposes of the present invention the terms “polymorphic position”, “polymorphic site”, “polymorphic locus”, and “polymorphic allele” shall be construed to be equivalent and are defined as the location of a sequence identified as having more than one nucleotide represented at that location in a population comprising at least one or more individuals, and/or chromosomes.

The term “isolated” is used herein to indicate that the material in question exists in a physical milieu distinct from that in which it occurs in nature, and thus is altered “by the hand of man” from its natural state.

As used herein, the term “polynucleotide” refers to a molecule comprising a nucleic acid of the invention. A polynucleotide can contain the nucleotide sequence of a full length cDNA sequence, including the 5′ and 3′ untranslated sequences, the coding region, with or without a signal sequence, the secreted protein coding region, and a genomic sequence with or without the accompanying promoter and transcriptional termination sequences, as well as fragments, epitopes, domains, and variants of the nucleic acid sequence. In specific examples, the polynucleotides of the invention include, among others, SEQ ID NOs: 1, 2, 11, and 12. As used herein, a “polypeptide” refers to a molecule having the translated amino acid sequence generated from the polynucleotide as defined.

On one hand, and in specific embodiments, the polynucleotides of the invention are at least 15, at least 30, at least 50, at least 100, at least 125, at least 500, or at least 1000 continuous nucleotides, but are less than or equal to 300 kb, 200 kb, 100 kb, 50 kb, 15 kb, 10 kb, 7.5 kb, 5 kb, 2.5 kb, 2.0 kb, or 1 kb in length. In further embodiments, the polynucleotides of the invention comprise a portion of the coding sequences, as disclosed herein, and can comprise all or a portion of one or more introns. In another embodiment, the polynucleotides preferentially do not contain the genomic sequence of the gene or genes flanking the human CYP3A5 and/or IPF-1 gene (i.e., 5′ or 3′ to the CYP3A5 and/or IPF-1 gene in the genome). In other embodiments, the polynucleotides of the invention do not contain the coding sequence of more than 1000, 500, 250, 100, 50, 25, 20, 15, 10, 5, 4, 3, 2, or 1 genomic flanking gene(s).

On the other hand, and in specific embodiments, the polynucleotides of the invention are at least 15, at least 30, at least 50, at least 100, at least 125, at least 500, or at least 1000 continuous nucleotides, but are less than or equal to 300 kb, 200 kb, 100 kb, 50 kb, 15 kb, 10 kb, 7.5 kb, 5 kb, 2.5 kb, 2.0 kb, or 1 kb, in length. In further embodiments, the polynucleotides of the invention comprise a portion of the coding sequences, comprise a portion of the non-coding sequences, comprise a portion of one or more intron sequences, etc., or any combination thereof, as disclosed herein. Alternatively, the polynucleotides of the invention can comprise the entire coding sequence, the entire 5′ non-coding sequence, the entire 3′ non-coding sequence, the entire sequence of one or more introns, the entire sequence of one or more exons, or any combination thereof, as disclosed herein. In another embodiment, the polynucleotides may correspond to a genomic sequence flanking a gene (i.e., 5′ or 3′to the gene of interest in the genome). In other embodiments, the polynucleotides of the invention may contain the non-coding sequence of more than 1000, 500, 250, 100, 50, 25, 20, 15, 10, 5, 4, 3, 2, or 1 genomic flanking gene(s).

A “polynucleotide” of the present invention also includes those polynucleotides capable of hybridizing, under stringent hybridization conditions, to sequences described herein, or the complement thereof. “Stringent hybridization conditions” refers to an overnight incubation at 42° C. in a solution comprising 50% formamide, 5×SSC (750 mM NaCl, 75 mM trisodium citrate), 50 mM sodium phosphate (pH 7.6), 5×Denhardt's solution, 10% dextran sulfate, and 20 μg/ml denatured, sheared salmon sperm DNA, followed by washing the filters in 0.1×SSC at about 65° C.

A “polynucleotide” of the present invention can be composed of any polyribonucleotide or polydeoxyribonucleotide, which can be unmodified RNA or DNA or modified RNA or DNA. For example, polynucleotides can be composed of single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, the polynucleotide can be composed of triple-stranded regions comprising RNA or DNA or both RNA and DNA. A polynucleotide can also contain one or more modified bases or DNA or RNA backbones modified for stability or for other reasons. “Modified” bases include, for example, tritylated bases and unconventional bases such as inosine. A variety of modifications can be made to DNA and RNA; thus, the term “polynucleotide” embraces chemically, enzymatically, or metabolically modified forms.

Unless otherwise indicated, all nucleotide sequences determined by sequencing a DNA molecule herein were determined using an automated DNA sequencer (such as the Model 3730-XL from Applied Biosystems, Inc., and/or ther PE 9700 from Perkin Elmer), and all amino acid sequences of polypeptides encoded by DNA molecules determined herein were predicted by translation of a DNA sequence determined above. The nucleotide sequence can also be determined by other approaches including manual DNA sequencing methods well known in the art. As is also known in the art, a single insertion or deletion in a determined nucleotide sequence compared to the actual sequence will cause a frame shift in translation of the nucleotide sequence such that the predicted amino acid sequence encoded by a determined nucleotide sequence will be completely different from the amino acid sequence actually encoded by the sequenced DNA molecule, beginning at the point of such an insertion or deletion. Since the present invention relates to the identification of single nucleotide polymorphisms whereby the novel sequence differs by as few as a single nucleotide from a reference sequence, identified SNPs were multiply verified to ensure each novel sequence represented a true SNP.

Using the information provided herein, a nucleic acid molecule of the present invention encoding a polypeptide of the present invention may be obtained using standard cloning and screening procedures, such as those for cloning cDNAs using mRNA as starting material.

The term “organism” as referred to herein is meant to encompass any organism referenced herein, though preferably meant to encompass eukaryotic organisms, more preferably meant to encompass mammals, and most preferably meant to encompass humans.

As used herein the terms “modulate” or “modulates” refer to an increase or decrease in the amount, quality or effect of a particular activity, DNA, RNA, or protein. The definition of “modulate” or “modulates” as used herein is meant to encompass agonists and/or antagonists of a particular activity, DNA, RNA, or protein.

The phrase “favorable response to a DPP-IV inhibitor” and the like, is meant to encompass a significant decrease in mean HbA1c levels post administration of a DPP-IV inhibitor, such as, for example, a decrease of at least about 0.6, and preferably a decrease of at least about 1.0, and more preferably a decrease of at least about 1.5 or more of HbA1c levels. The HbA1c units are reported as a standard unit, % HbA1c, as described in Colman et al., “Glycohaemoglobin—a crucial measurement in modern diabetes management. Progress towards standardization and improved precision of measurement”, Consensus Statement from the Australian Diabetes Society, Royal College of Australia and Australian Association of Clinical Biochemists, pp 1-11.

The terms “7068A” and “T7068A” are meant to refer to the “A” allele at the polymorphic locus located at nucleotide 7068 of SEQ ID NO:2. One skilled in the art would recognize that reference to this allele is not limited to only SEQ ID NO:2, but rather necessarily also includes any other polynucleotide that may include this sequence, or a portion of this sequence surrounding this polymorphic locus, on account of SEQ ID NO:2 merely representing a small portion of chromosome 7 encoding the CYP3A5 gene.

The term “G7068” is meant to refer to the “G” allele at the polymorphic locus located at nucleotide 7068 of SEQ ID NO:1. One skilled in the art would recognize that reference to this allele is not limited to only SEQ ID NO:1, but rather necessarily also includes any other polynucleotide that may include this sequence, or a portion of this sequence surrounding this polymorphic locus, on account of SEQ ID NO:1 merely representing a small portion of chromosome 7 encoding the CYP3A5 gene.

The terms “4445T” and “C4445T” are meant to refer to the “T” allele at the polymorphic locus located at nucleotide 4445 of SEQ ID NO:12. One skilled in the art would recognize that reference to this allele is not limited to only SEQ ID NO:12, but rather necessarily also includes any other polynucleotide that may include this sequence, or a portion of this sequence surrounding this polymorphic locus, on account of SEQ ID NO:12 merely representing a small portion of chromosome 13 encoding the IPF-1 gene.

The term “C4445” is meant to refer to the “C” allele at the polymorphic locus located at nucleotide 4445 of SEQ ID NO:11. One skilled in the art would recognize that reference to this allele is not limited to only SEQ ID NO:11, but rather necessarily also includes any other polynucleotide that may include this sequence, or a portion of this sequence surrounding this polymorphic locus, on account of SEQ ID NO:11 merely representing a small portion of chromosome 13 encoding the IPF-1 gene.

### Polynucleotides and Polypeptides of the Invention

### Features of Gene No:1

The present invention relates to isolated nucleic acid molecules comprising all or a portion of one or more alleles of SNP1 of the human CYP3A5 gene, as provided in FIGS. 1A-L (SEQ ID NO:1) comprising at least one polymorphic locus. The allele described for the SNP1 in FIGS. 1A-L (SEQ ID NO:1) represents the reference allele for this SNP and is exemplified by a “G” at nucleotide position 7068. Fragments of this polynucleotide are at least about 10 nucleotides, at least about 20 nucleotides, at least about 40 nucleotides, or at least about 100 contiguous nucleotides and comprise one or more reference alleles at the nucleotide position(s) provided in FIGS. 1A-L (SEQ ID NO:1).

In one embodiment, the invention relates to a method for predicting the likelihood that an individual will have an increased likelihood of achieving a favorable response to a pharmaceutically acceptable amount of a DPP-IV inhibitor, particularly for individuals of Hispanic descent, comprising the step of identifying the nucleotide present at nucleotide position 7068 of SEQ ID NO:1, from a DNA sample to be assessed, or the corresponding nucleotide at this position if only a fragment of the sequence provided as SEQ ID NO:1 is assessed. The presence of the reference allele at said position indicates that the individual from whom said DNA sample or fragment was obtained has a decreased likelihood of achieving a favorable response to a pharmaceutically acceptable amount of a DPP-IV inhibitor than an individual having the variant allele(s) at said position(s).

Importantly, the presence of the reference allele at said position in a nucleic acid sample provided by an individual, indicates that said individual may require the administration of a correspondingly higher amount of a DPP-IV inhibitor relative to another individual having the variant allele(s) at said position. Therefore, such individuals may require the level of administered DPP-IV inhibitor to be “titrated-up” to achieve a more favorable response.

Representative disorders that can be detected, diagnosed, identified, treated, prevented, and/or ameliorated by the SNPs and methods of the present invention include, but are not limited to, the following diseases and disorders: DPP-IV abnormalities, susceptibility to developing DPP-IV abnormalities, diabetes, disorders associated with aberrant CYP3A5 expression, disorders associated with aberrant CYP3A5 regulation, disorders associated with aberrant CYP3A5 activity, disorders associated with aberrant HbA1c levels, disorders associated with elevated HbA1c plasma/serum levels, diabetes, type II diabetes, complications of diabetes, including retinopathy, neuropathy, nephropathy and delayed wound healing, diseases related to diabetes including insulin resistance, impaired glucose homeostatis, hyperglycaemia, hyperinsulinemia, elevated blood levels of fatty acids or glycerol, obesity, hyperlipidemia including hypertriglyceridemia, Syndrome X, atherosclerosis, and hypertension, among others.

### Features of Gene No:2

The present invention relates to isolated nucleic acid molecules comprising all or a portion of one or more alleles of SNP1 of the human CYP3A5 gene, as provided in FIGS. 2A-L (SEQ ID NO:2) comprising at least one polymorphic locus. The allele described for SNP1 in FIGS. 2A-L (SEQ ID NO:2) represents the variant allele for this SNP and is exemplified by an “A” at nucleotide position 7068. Fragments of this polynucleotide are at least about 10 nucleotides, at least about 20 nucleotides, at least about 40 nucleotides, at least about 100 contiguous nucleotides and comprise one or more variant alleles at the nucleotide position(s) provided in FIGS. 2A-L (SEQ ID NO:2).

In one embodiment, the invention relates to a method for predicting the likelihood that an individual will have a favorable response to the administration of a pharmaceutically acceptable amount of a DPP-IV inhibitor, particularly for individuals of Hispanic descent, comprising the step of identifying the nucleotide present at nucleotide position 7068 of SEQ ID NO:2, from a DNA sample to be assessed, or the corresponding nucleotide at this position if only a fragment of the sequence provided as SEQ ID NO:2 is assessed. The presence of the variant allele at said position indicates that the individual from whom said DNA sample or fragment was obtained has an increased likelihood of achieving a favorable response to a pharmaceutically acceptable amount of a DPP-IV inhibitor, compared to an individual having the reference allele(s) at said position(s).

Importantly, the presence of the variant allele at said position in a DNA sample provided by an individual indicates that said individual may have an increased likelihood of achieving a favorable response to a DPP-IV inhibitor and that the typical dose may be sufficient relative to another individual having the reference allele(s) at said position. In addition, the presence of the variant allele at said position in a DNA sample can indicate that a lower dose of DPP-IV inhibitor may still enable the patient to achieve a favorable response to the administration of a pharmaceutically acceptable amount of a DPP-IV inhibitor.

Representative disorders that can be detected, diagnosed, identified, treated, prevented, and/or ameliorated by the SNPs and methods of the present invention include, but are not limited to, the following diseases and disorders: DPP-IV abnormalities, susceptibility to developing DPP-IV abnormalities, diabetes, disorders associated with aberrant CYP3A5 expression, disorders associated with aberrant CYP3A5 regulation, disorders associated with aberrant CYP3A5 activity, disorders associated with aberrant HbA1c levels, disorders associated with elevated HbA1c plasma/serum levels, diabetes, type II diabetes, complications of diabetes, including retinopathy, neuropathy, nephropathy and delayed wound healing, diseases related to diabetes including insulin resistance, impaired glucose homeostatis, hyperglycaemia, hyperinsulinemia, elevated blood levels of fatty acids or glycerol, obesity, hyperlipidemia including hypertriglyceridemia, Syndrome X, atherosclerosis, and hypertension, among others.

### Features of Gene No:3

The present invention relates to isolated nucleic acid molecules comprising all or a portion of one or more alleles of SNP1 of the human IPF-1 gene, as provided in FIGS. 4A-D (SEQ ID NO:11) comprising at least one polymorphic locus. The allele described for SNP1 in FIGS. 4A-D (SEQ ID NO:11) represents the reference allele for this SNP and is exemplified by a “C” at nucleotide position 4445. Fragments of this polynucleotide are at least about 10 nucleotides, at least about 20 nucleotides, at least about 40 nucleotides, or at least about 100 contiguous nucleotides and comprise one or more reference alleles at the nucleotide position(s) provided in FIGS. 4A-D (SEQ ID NO:11).

In one embodiment, the invention relates to a method for predicting the likelihood that an individual will have an increased likelihood of achieving a favorable response to a pharmaceutically acceptable amount of a DPP-IV inhibitor, particularly for individuals of Hispanic descent, comprising the step of identifying the nucleotide present at nucleotide position 4445 of SEQ ID NO:11, from a DNA sample to be assessed, or the corresponding nucleotide at this position if only a fragment of the sequence provided as SEQ ID NO:11 is assessed. The presence of the reference allele at said position indicates that the individual from whom said DNA sample or fragment was obtained has a decreased likelihood of achieving a favorable response to a pharmaceutically acceptable amount of a DPP-IV inhibitor than an individual having the variant allele(s) at said position(s).

Importantly, the presence of the reference allele at said position in a nucleic acid sample provided by an individual, indicates that said individual may require the administration of a correspondingly higher amount of a DPP-IV inhibitor relative to another individual having the variant allele(s) at said position. Therefore, such individuals may require the level of administered DPP-IV inhibitor to be “titrated-up” to achieve a more favorable response.

Representative disorders that can be detected, diagnosed, identified, treated, prevented, and/or ameliorated by the SNPs and methods of the present invention include, but are not limited to, the following diseases and disorders: DPP-IV abnormalities, susceptibility to developing DPP-IV abnormalities, diabetes, disorders associated with aberrant IPF1 expression, disorders associated with aberrant IPF1 regulation, disorders associated with aberrant IPF1 activity, disorders associated with aberrant HbA1c levels, disorders associated with elevated HbA1c plasma/serum levels, diabetes, type II diabetes, complications of diabetes, including retinopathy, neuropathy, nephropathy and delayed wound healing, diseases related to diabetes including insulin resistance, impaired glucose homeostatis, hyperglycaemia, hyperinsulinemia, elevated blood levels of fatty acids or glycerol, obesity, hyperlipidemia including hypertriglyceridemia, Syndrome X, atherosclerosis, and hypertension, among others.

### Features of Gene No:4

The present invention relates to isolated nucleic acid molecules comprising all or a portion of one or more alleles of SNP1 of the human IPF1 gene, as provided in FIGS. 5A-D (SEQ ID NO:12) comprising at least one polymorphic locus. The allele described for SNP1 in FIGS. 4A-D (SEQ ID NO:12) represents the variant allele for this SNP and is exemplified by a “T” at nucleotide position 4445. Fragments of this polynucleotide are at least about 10 nucleotides, at least about 20 nucleotides, at least about 40 nucleotides, at least about 100 contiguous nucleotides and comprise one or more variant alleles at the nucleotide position(s) provided in FIGS. 5A-D (SEQ ID NO:12).

In one embodiment, the invention relates to a method for predicting the likelihood that an individual will have favorable response to the administration of a pharmaceutically acceptable amount of a DPP-IV inhibitor, particularly for individuals of Hispanic descent, comprising the step of identifying the nucleotide present at nucleotide position 4445 of SEQ ID NO:12, from a DNA sample to be assessed, or the corresponding nucleotide at this position if only a fragment of the sequence provided as SEQ ID NO:12 is assessed. The presence of the variant allele at said position indicates that the individual from whom said DNA sample or fragment was obtained has an increased likelihood of achieving a favorable response to a pharmaceutically acceptable amount of a DPP-IV inhibitor, compared to an individual having the reference allele(s) at said position(s).

Importantly, the presence of the variant allele at said position in a DNA sample provided by an individual indicates that said individual may have an increased likelihood of achieving a favorable response to a DPP-IV inhibitor and that the typical dose may be sufficient relative to another individual having the reference allele(s) at said position. In addition, the presence of the variant allele at said position in a DNA sample may indicate that a lower dose of DPP-IV inhibitor can enable the patient to achieve a favorable response to the administration of a pharmaceutically acceptable amound of a DPP-IV inhibitor.

Representative disorders that can be detected, diagnosed, identified, treated, prevented, and/or ameliorated by the SNPs and methods of the present invention include, but are not limited to, the following diseases and disorders: DPP-IV abnormalities, susceptibility to developing DPP-IV abnormalities, diabetes, disorders associated with aberrant IPF-1 expression, disorders associated with aberrant IPF-1 regulation, disorders associated with aberrant IPF-1 activity, disorders associated with aberrant HbA1c levels, disorders associated with elevated HbA1c plasma/serum levels, diabetes, type II diabetes, complications of diabetes, including retinopathy, neuropathy, nephropathy and delayed wound healing, diseases related to diabetes including insulin resistance, impaired glucose homeostatis, hyperglycaemia, hyperinsulinemia, elevated blood levels of fatty acids or glycerol, obesity, hyperlipidemia including hypertriglyceridemia, Syndrome X, atherosclerosis, and hypertension, among others.

TABLE I

| Poly- | | | Polymorphic | Nucleotide | Nucleotide at | SEQ |
|---|---|---|---|---|---|---|
| nucleotide | CDNA | | Locus | Position of | Polymorphic | ID |
| No. | CloneID | Allele | Number | Polymorphic Locus | Locus | NO: |
| 1 | Human CYP3A5 | Reference | 1 | 7068 | G | 1 |
| | Gene - SNP1 | | | | | |
| 2 | Human CYP3A5 | Variable | 1 | 7068 | A | 2 |
| | Gene - SNP1 | | | | | |
| 3 | Human IPF1 | Reference | 1 | 4445 | C | 11 |
| | Gene - SNP1 | | | | | |
| 4 | Human IPF1 | Variable | 1 | 4445 | T | 12 |
| | Gene - SNP1 | | | | | |

The present invention provides a polynucleotide comprising the sequence identified as SEQ ID NOs:1 and 2 for the CYP3A5 gene, and SEQ ID NOs:11 and 12 for the IPF-1 gene; or a fragment containing the polymorphic allele, wherein said fragment comprises at least 10 contiguous nucleotides of SEQ ID NO:1 and/or 2 for the CYP3A5 gene, and/or SEQ ID NO:11 and/or 12 for the IPF-1 gene.

Preferably, the present invention is directed to a polynucleotide comprising the sequence identified as SEQ ID NO:1 and/or 2 for the CYP3A5 gene, and/or SEQ ID NO:11 and/or 12 for the IPF1 gene, that is less than, or equal to, a polynucleotide sequence that is 5 mega basepairs, 1 mega basepairs, 0.5 mega basepairs, 0.1 mega basepairs, 50,000 basepairs, 20,000 basepairs, or 10,000 basepairs in length.

The present invention encompasses polynucleotides with sequences complementary to those of the polynucleotides of the present invention disclosed herein. Such sequences can be complementary to the sequence disclosed as SEQ ID NO:1 and/or 2 for the CYP3A5 gene, and/or SEQ ID NO:11 and/or 12 for the IPF-1 gene.

The invention encompasses the application of Polymerase Chain Reaction (PCR) methodology to the polynucleotide sequences of the present invention, and/or the cDNA encoding the polypeptides of the present invention. PCR techniques for the amplification of nucleic acids are described in U.S. Pat. No. 4,683,195 and Saiki et al., 1988, Science, 239:487-491. PCR, for example, may include the following steps, of denaturation of template nucleic acid (if double-stranded), annealing of primer to target, and polymerization. The nucleic acid probed or used as a template in the amplification reaction can be genomic DNA, cDNA, RNA, or a PNA. PCR can be used to amplify specific sequences from genomic DNA, specific RNA sequence, and/or cDNA transcribed from mRNA. References for the general use of PCR techniques, including specific method parameters, include Mullis et al., 1987, Cold Spring Harbor Symp. Quant. Biol., 51:263; Ehrlich (ed), PCR Technology, Stockton Press, NY, 1989; Ehrlich et al., 1991, Science, 252:1643-1650; and “PCR Protocols, A Guide to Methods and Applications”, Eds., Innis et al., Academic Press, New York, (1990).

### Polynucleotide Variants

The present invention also encompasses variants (e.g., allelic variants, orthologs, etc.) of the polynucleotide sequence disclosed herein in SEQ ID NO:1 and/or 2 for the CYP3A5 gene, and/or SEQ ID NO:11 and/or 12 for the IPF1 gene, and the complementary strand thereto.

The present invention also encompasses variants of the polypeptide sequence, and/or fragments therein, disclosed in SEQ ID NO:1 and/or 2 for the CYP3A5 gene, and/or SEQ ID NO:11 and/or 12 for the IPF1 gene.

“Variant” refers to a polynucleotide or polypeptide differing from the polynucleotide or polypeptide of the present invention, but retaining essential properties thereof. Generally, variants are overall closely similar, and, in many regions, identical to the polynucleotide or polypeptide of the present invention.

In another embodiment, the invention encompasses nucleic acid molecules which comprise a polynucleotide which hybridizes under stringent conditions, or alternatively, under lower stringency conditions, to a polynucleotide described above. Polynucleotides which hybridize to the complement of these nucleic acid molecules under stringent hybridization conditions or alternatively, under lower stringency conditions, are also encompassed by the invention, as are polypeptides encoded by these polypeptides.

### Polynucleotide Fragments

The present invention is directed to polynucleotide fragments of the polynucleotides of the invention, and polynucleotide sequences that hybridize thereto.

In the present invention, a “polynucleotide fragment” refers to a short polynucleotide having a nucleic acid sequence which is a portion of that shown in SEQ ID NO:1 and/or 2 for the CYP3A5 gene, and/or SEQ ID NO:11 and/or 12 for the IPF-1 gene, or the complementary strand thereto. The nucleotide fragments of the invention are preferably at least about 15 nucleotides, and more preferably at least about 20 nucleotides, still more preferably at least about 30 nucleotides, and even more preferably, at least about 40 nucleotides, at least about 50 nucleotides, at least about 75 nucleotides, or at least about 150 nucleotides in length, and comprise at least one polymorphic locus. A fragment “at least 20 nucleotide in length,” for example, is intended to include 20 or more contiguous nucleotides from the cDNA sequence shown in SEQ ID NO:1 and/or 2 for the CYP3A5 gene, and/or SEQ ID NO:11 and/or 12 for the IPF1 gene. In this context “about” includes the particularly recited value, a value larger or smaller by several (5, 4, 3, 2, or 1) nucleotides, at either terminus, or at both termini. These nucleotide fragments have uses that include, but are not limited to, diagnostic probes and primers as discussed herein. Of course, larger fragments (e.g., 50, 150, 500, 600, 2000 nucleotides) are also preferred.

Moreover, representative examples of polynucleotide fragments of the invention, include, for example, isolated fragments comprising, or alternatively consisting of, a sequence from about nucleotide number 1-50, 51-100, 101-150, 151-200, 201-250, 251-300, 301-350, 351-400, 401-450, 451-500, 501-550, 551-600, 651-700, 701-750, 751-800, 800-850, 851-900, 901-950, 951-1000, 1001-1050, 1051-1100, 1101-1150, 1151-1200, 1201-1250, 1251-1300, 1301-1350, 1351-1400, 1401-1450, 1451-1500, 1501-1550, 1551-1600, 1601-1650, 1651-1700, 1701-1750, 1751-1800, 1801-1850, 1851-1900, 1901-1950, 1951-2000, or 2001 to the end of SEQ ID NO:1 and/or 2 for the CYP3A5 gene, and/or SEQ ID NO:11 and/or 12 for the IPF1 gene, or the complementary strand thereto. In this context “about” includes the particularly recited ranges, and ranges larger or smaller by several (5, 4, 3, 2, or 1) nucleotides, at either terminus or at both termini. Preferably, these fragments encode a polypeptide which has biological activity. More preferably, these polynucleotides can be used as probes or primers as discussed herein. Also encompassed by the present invention are polynucleotides which hybridize to these nucleic acid molecules under stringent hybridization conditions or lower stringency conditions, as are the polypeptides encoded by these polynucleotides.

### Kits

The invention further provides kits comprising at least one agent for identifying which alleleic form of the SNPs identified herein is present in a sample. For example, suitable kits can comprise at least one antibody specific for a particular protein or peptide encoded by one alleleic form of the gene, or allele-specific oligonucleotide as described herein. Often, the kits contain one or more pairs of allele-specific oligonucleotides hybridizing to different forms of a polymorphism. In some kits, the allele-specific oligonucleotides are provided immobilized to a substrate. For example, the same substrate can comprise allele-specific oligonucleotide probes for detecting at least 1, 10, 100 or all of the polymorphisms shown in Table I. Optional additional components of the kit include, for example, reagents, buffers, restriction enzymes, reverse-transcriptase or polymerase, the substrate nucleoside triphosphates, means used to label (for example, an avidin-enzyme conjugate and enzyme substrate and chromogen if the label is biotin, fluophores, and others as described herein), and the appropriate buffers for reverse transcription, PCR, or hybridization reactions. Usually, the kit also contains instructions for carrying out the methods.

The present invention provides kits that can be used in the methods described herein. In one embodiment, a kit comprises a single primer or probe of the invention comprising a means to detect at least one polymorphic locus, said means preferably comprises a purified primer or probe, in one or more containers. Such a primer or probe can further comprise a detectable label such as a fluorescent compound, an enzymatic substrate, a radioactive compound, a luminescent compound, a fluorophore, and/or a fluorophore linked to a terminator contained therein. Such a kit can further comprise reagents required to enable adequate hybridization of said single primer or probe to a DNA test sample, such that under suitable conditions, the primer or probe is capable of binding to said DNA test sample and signaling whether the variant or reference allele at the polymorphic locus is present in said DNA test sample.

In one example, the kit comprises a means for detecting the presence of a polymorphic locus comprising one specific allele of at least one polynucleotide in a DNA test sample which serves as a template nucleic acid comprising: (a) forming an oligonucleotide bound to the polymorphic locus wherein the oligonucleotide comprises a fluorophore linked to a terminator contained therein; and (b) detecting fluorescence polarization of the fluorophore of the fluorescently-labeled oligonucleotide, wherein the oligonucleotide is formed from a primer bound to said DNA sample immediately 3′ to the polymorphic locus and a terminator covalently linked to a fluorophore, and wherein said terminator-linked fluorophore binds to the polymorphic locus and reacts with the primer to produce an extended primer which is said fluorescently labeled oligonucleotide, wherein an increase in fluorescence polarization indicates the presence of the specific allele at the polymorphic locus, thereby detecting the presence of the specific allele at the polymorphic locus by said increase in fluorescence polarization.

The kit of the present invention may comprise the following non-limiting examples of fluorophores linked to a primer or probe of the present invention: 5-carboxyfluorescein (FAM-ddNTPs); 6-carboxy-X-rhodamine (ROX-ddNTPs); N,N,N′,N′-tetramethyl-6-carboxyrhodamine (TMR-ddNTPs); and BODIPY-Texas Red (BTR-ddNTPs).

The present invention is also directed towards a kit comprising a solid support to which oligonucleotides comprising at least 10 contiguous nucleotides of SEQ ID NO:1 or 2 for the CYP3A5 gene, and/or SEQ ID NO:11 or 12 for the IPF-1 gene wherein said oligonucleotide further comprises at least one polymorphic locus of SEQ ID NO:1 or 2 for the CYP3A5 gene, and/or SEQ ID NO:11 or 12 for the IPF1 gene, are affixed. In such an embodiment, detection of a polynucleotide within a sample comprising the same or similar sequence to said oligonucleotide can be detected by hybridization.

The solid surface reagent in the above assay is prepared by known techniques for attaching protein material to solid support material, such as polymeric beads, dip sticks, 96-well plate or filter material. These attachment methods generally include non-specific adsorption of the oligonucleotide to the support or covalent attachment of the oligonucleotide to a chemically reactive group on the solid support. Alternatively, streptavidin coated plates can be used in conjunction with biotinylated oligonucleotide(s).

Thus, the invention provides an assay system or kit for carrying out this diagnostic method. The kit generally includes a support with surface-bound oligonucleotides, and a reporter for detecting hybridization of said oligonucleotide to a test polynucleotide.

Methods of Using The Allelic Polynucleotides of the Present Invention

The determination of the polymorphic form(s) present in an individual at one or more polymorphic sites defined herein can be used in a number of methods.

In preferred embodiments, the polynucleotides and polypeptides of the present invention, including allelic and variant forms thereof, have uses which include, but are not limited to diagnosing individuals to identify whether a given individual has an increased likelihood of achieving a favorable response to the administration of a pharmaceutically acceptable amount of a DPP-IV inhibitor using the genotype assays of the present invention.

In preferred embodiments, the polynucleotides and polypeptides of the present invention, including allelic and variant forms thereof, have uses which include, but are not limited to diagnosing individuals to identify whether a given individual has an increased likelihood of achieving a favorable response to the administration of a pharmaceutically acceptable amount of a DPP-IV inhibitor. For those individuals predicted to have a lower likelihood of achieving a favorable response, an increased dosage of a DPP-IV inhibitor may be warranted. Such a higher level of a pharmaceutically acceptable dose of a DPP-IV inhibitor for a patient identified as having a lower likelihood of achieving a favorable response may be, for example, about 5%, 10%, 15%, 20%, 25%, 30%, 35%, 40%, 45%, 50%, 55%, 60%, 65%, 75%, 80%, 85%, 90%, or 95% higher, or 1.5-, 2-, 2.5-, 3-, 3.5-, 4-, 4,5-, or even 5-fold higher than the prescribed or typical dose, as may be the case.

In another embodiment, the polynucleotides and polypeptides of the present invention, including allelic and variant forms thereof, either alone, or in combination with other polymorphic polynucleotides (haplotypes) are useful as genetic markers for predicting whether an individual has an increased likelihood of achieving a favorable response to the administration of a pharmaceutically acceptable amount of a DPP-IV inhibitor.

Additionally, the polynucleotides and polypeptides of the present invention, including allelic and/or variant forms thereof, are useful for creating additional antagonists directed against these polynucleotides and polypeptides, which include, but are not limited to the design of antisense RNA, ribozymes, PNAs, recombinant zinc finger proteins (Wolfe et al., 2000, Structure Fold Des. 8:739-50; Kang et al., 2000, J. Biol, Chem. 275:8742-8; Wang et al., 1999, Proc. Natl. Acad. Sci. U.S.A. 96:9568-73; McColl et al., 1999, Proc. Natl. Acad. Sci. U.S.A. 96:9521-6; Segal et al., 1999, Proc. Natl. Acad. Sci. U.S.A. 96:2758-63; Wolfe et al., 1998, J. Molec. Biol. 285:1917-34; Pomerantz et al., 1998, Biochemistry 37:965-70; Leon et al., 2000, Biol. Res. 33:21-30; Berg et al., 1997, Ann. Rev. Biophys. Biomol. Struct. 26:357-71), in addition to other types of antagonists which are either described elsewhere herein, or known in the art.

The polynucleotides and polypeptides of the present invention, including allelic and/or variant forms thereof, are useful for identifying small molecule antagonists directed against the variant forms of these polynucleotides and polypeptides, preferably wherein such small molecules are useful as therapeutic and/or pharmaceutical compounds for the treatment, detection, prognosis, and/or prevention of the following, nonlimiting diseases and/or disorders: DPP-IV abnormalities, susceptibility to developing DPP-IV abnormalities, diabetes, disorders associated with aberrant CYP3A5 expression, disorders associated with aberrant CYP3A5 regulation, disorders associated with aberrant CYP3A5 activity, disorders associated with aberrant IPF-1 expression, disorders associated with aberrant IPF-1 regulation, disorders associated with aberrant IPF-1 activity, disorders associated with aberrant HbA1c levels, disorders associated with elevated HbA1c plasma/serum levels, diabetes, type II diabetes, complications of diabetes, including retinopathy, neuropathy, nephropathy and delayed wound healing, diseases related to diabetes including insulin resistance, impaired glucose homeostatis, hyperglycaemia, hyperinsulinemia, elevated blood levels of fatty acids or glycerol, obesity, hyperlipidemia including hypertriglyceridemia, Syndrome X, atherosclerosis, and hypertension.

Additional disorders which can be detected, diagnosed, identified, treated, prevented, and/or ameliorated by the SNPs and methods of the present invention include, the following, non-limiting diseases and disorders: diabetic related diseases such as insulin resistance, hyperglycemia, obesity, inflammation, dysmetabolic syndrome, and related diseases. Additional uses of the polynucleotides and polypeptides of the present invention are provided herein.

### Modified Polypeptides and Gene Sequences

The invention further provides variant forms of nucleic acids and corresponding proteins. The nucleic acids comprise one of the sequences described in Table I, in which the polymorphic position is occupied by one of the alternative bases for that position. Some nucleic acids encode full-length variant forms of proteins. Variant genes can be expressed in an expression vector in which a variant gene is operably linked to a native or other promoter. Usually, the promoter is a eukaryotic promoter for expression in a mammalian cell. The transcription regulation sequences typically include a heterologous promoter and optionally an enhancer which is recognized by the host. The selection of an appropriate promoter, for example trp, lac, phage promoters, glycolytic enzyme promoters and tRNA promoters, depends on the host selected. Commercially available expression vectors can be used. Vectors can include host-recognized replication systems, amplifiable genes, selectable markers, host sequences useful for insertion into the host genome, and the like.

The means of introducing the expression construct into a host cell varies depending upon the particular construction and the target host. Suitable means include fusion, conjugation, transfection, transduction, electroporation or injection, as described in Sambrook, supra. A wide variety of host cells can be employed for expression of the variant gene, both prokaryotic and eukaryotic. Suitable host cells include bacteria such as E. coli, yeast, filamentous fungi, insect cells, mammalian cells, typically immortalized, e.g., mouse, CHO, human and monkey cell lines and derivatives thereof. Preferred host cells are able to process the variant gene product to produce an appropriate mature polypeptide. Processing includes glycosylation, ubiquitination, disulfide bond formation, general post-translational modification, and the like. As used herein, “gene product” includes mRNA, peptide and protein products.

The protein may be isolated by conventional means of protein biochemistry and purification to obtain a substantially pure product, i.e., 80%, 95%, or 99% free of cell component contaminants, as described in Jacoby, Methods in Enzymology Volume 104, Academic Press, New York (1984); Scopes, Protein Purification, Principles and Practice, 2nd Edition, Springer-Verlag, New York (1987); and Deutscher (ed), Guide to Protein Purification, Methods in Enzymology, Vol. 182 (1990). If the protein is secreted, it can be isolated from the supernatant in which the host cell is grown. If not secreted, the protein can be isolated from a lysate of the host cells.

### Haplotype Based Genetic Analysis

The invention further provides methods for applying the polynucleotides of the present invention to the elucidation of haplotypes. Such haplotypes can be associated with any one or more of the disease conditions referenced elsewhere herein. A “haplotype” is defined as the pattern of a set of alleles of single nucleotide polymorphisms along a chromosome. For example, consider the case of three single nucleotide polymorphisms (SNP1, SNP2, and SNP3) in one chromosome region, of which SNP1 is an A/G polymorphism, SNP2 is a G/C polymorphism, and SNP3 is an A/C polymorphism. A and G are the alleles for the first, G and C for the second, and A and C for the third SNP. Given two alleles for each SNP, there are three possible genotypes for individuals at each SNP. For example, for the first SNP, A/A, A/G and G/G are the possible genotypes for individuals. When an individual has a genotype for a SNP in which the alleles are not the same, for example A/G for the first SNP, then the individual is a heterozygote. When an individual has an A/G genotype at SNP1, G/C genotype at SNP2, and A/C genotype at SNP3, there are four possible combinations of haplotypes (A, B, C, and D) for this individual. The set of SNP genotypes of this individual alone would not provide sufficient information to resolve which combination of haplotypes this individual possesses. However, when this individual's parents' genotypes are available, haplotypes could then be assigned unambiguously. For example, if one parent had an A/A genotype at SNP1, a G/C genotype at SNP2, and an A/A genotype at SNP3, and the other parent had an A/G genotype at SNP1, C/C genotype at SNP2, and C/C genotype at SNP3, while the child was a heterozygote at all three SNPs, there is only one possible haplotype combination, assuming there was no crossing over in this region during meiosis.

When the genotype information of relatives is not available, haplotype assignment can be done using the long range-PCR method (Clark, 1990, Molec. Biol. Evol. 7: 111-22; Clark et al., 1998, Am J Hum Genet. 63: 595-612; Fullerton et al., 2000, Am J Hum. Genet. 67: 881-900; Templeton et al., 2000, Am J Hum Genet. 66: 69-83). When the genotyping result of the SNPs of interest are available from general population samples, the most likely haplotypes can also be assigned using statistical methods (Excoffier & Slatkin, 1995, Mol Biol Evol 12: 921-7; Fallin & Schork, 2000, Am J Hum Genet 67: 947-59; Long et al., 1995, Am J Hum Genet 56: 799-810).

Once an individual's haplotype in a certain chromosome region (i.e., locus) has been determined, it can be used as a tool for genetic association studies using different methods, which include, for example, haplotype relative risk analysis (Knapp et al., 1993, Am J Hum Genet 52: 1085-93; Li et al., 1998, Schizophr Res 32: 87-92; Matise, 1995, Genet Epidemiol 12: 641-5; Ott, J., 1989, Genet Epidemiol 6: 127-30; Terwilliger & =Ott, 1992, Hum Hered 42: 337-46). Haplotype based genetic analysis, using a combination of SNPs, provides increased detection sensitivity, and hence statistical significance, for genetic associations of diseases, as compared to analyses using individual SNPs as markers. Multiple SNPs present in a single gene or a continuous chromosomal region are useful for such haplotype-based analyses.

### Uses of the Polynucleotides

Each of the polynucleotides identified herein can be used in numerous ways as reagents. The following description should be considered exemplary and utilizes known techniques.

Increased or decreased expression of the gene in affected organisms as compared to unaffected organisms can be assessed using polynucleotides of the present invention. Any of these alterations, including altered expression, or the presence of at least one SNP of the present invention within the gene, can be used as a diagnostic or prognostic marker.

The invention provides a diagnostic method useful during diagnosis of a disorder, involving measuring the presence or expression level of polynucleotides of the present invention in cells or body fluid from an organism and comparing the measured gene expression level with a standard level of polynucleotide expression level, whereby an increase or decrease in the gene expression level compared to the standard is indicative of a disorder.

By “measuring the expression level of a polynucleotide of the present invention” is intended qualitatively or quantitatively measuring or estimating the level of the polypeptide of the present invention or the level of the mRNA encoding the polypeptide in a first biological sample either directly (e.g., by determining or estimating absolute protein level or mRNA level) or relatively (e.g., by comparing to the polypeptide level or mRNA level in a second biological sample). Preferably, the polypeptide level or mRNA level in the first biological sample is measured or estimated and compared to a standard polypeptide level or mRNA level, the standard being taken from a second biological sample obtained from an individual not having the disorder or being determined by averaging levels from a population of organisms not having a disorder. As will be appreciated in the art, once a standard polypeptide level or mRNA level is known, it can be used repeatedly as a standard for comparison.

By “biological sample” is intended any biological sample obtained from an organism, body fluids, cell line, tissue culture, or other source which contains the polypeptide of the present invention or mRNA. As indicated, biological samples include body fluids (such as the following non-limiting examples, sputum, amniotic fluid, urine, saliva, breast milk, secretions, interstitial fluid, blood, serum, spinal fluid, etc.) which contain the polypeptide of the present invention, and other tissue sources found to express the polypeptide of the present invention. Methods for obtaining tissue biopsies and body fluids from organisms are well known in the art. Where the biological sample is to include mRNA, a tissue biopsy is the preferred source.

The method(s) provided above can preferably be applied in a diagnostic method and/or kits in which polynucleotides and/or polypeptides are attached to a solid support. In one exemplary method, the support may be a “gene chip” or a “biological chip” as described in U.S. Pat. Nos. 5,837,832, 5,874,219, and 5,856,174. Further, such a gene chip with polynucleotides of the present invention attached can be used to identify polymorphisms between the polynucleotide sequences, with polynucleotides isolated from a test subject. The knowledge of such polymorphisms (i.e. their location, as well as, their existence) would be beneficial in identifying disease loci for many disorders, including proliferative diseases and conditions. Such a method is described in U.S. Pat. Nos. 5,858,659 and 5,856,104. The US patents referenced supra are hereby incorporated by reference in their entirety herein.

The present invention encompasses polynucleotides of the present invention that are chemically synthesized, or reproduced as peptide nucleic acids (PNA), or according to other methods known in the art. The use of PNAs would serve as the preferred form if the polynucleotides are incorporated onto a solid support, or gene chip. For the purposes of the present invention, a peptide nucleic acid (PNA) is a polyamide type of DNA analog and the monomeric units for adenine, guanine, thymine and cytosine are available commercially (Perceptive Biosystems). Certain components of DNA, such as phosphorus, phosphorus oxides, or deoxyribose derivatives, are not present in PNAs (as disclosed by Nielsen et al., 1991, Science 254: 1497 and Egholm et al., 1993, Nature 365: 666). PNAs bind specifically and tightly to complementary DNA strands and are not degraded by nucleases. In fact, PNA binds more strongly to DNA than DNA itself does. This is probably because there is no electrostatic repulsion between the two strands, and also the polyamide backbone is more flexible. Because of this, PNA/DNA duplexes bind under a wider range of stringency conditions than DNA/DNA duplexes, making it easier to perform multiplex hybridization. Smaller probes can be used than with DNA due to the stronger binding characteristics of PNA:DNA hybrids. In addition, it is more likely that single base mismatches can be determined with PNA/DNA hybridization because a single mismatch in a PNA/DNA 15-mer lowers the melting point (T.sub.m) by 8°-20° C., vs. 4°-16° C. for the DNA/DNA 15-mer duplex. Also, the absence of charge groups in PNA means that hybridization can be done at low ionic strengths and reduce possible interference by salt during the analysis.

Polynucleotides of the present invention are also useful in gene therapy. One goal of gene therapy is to insert a normal gene into an organism having a defective gene, in an effort to correct the genetic defect. The polynucleotides disclosed in the present invention offer a means of targeting such genetic defects in a highly accurate manner. Another goal is to insert a new gene that was not present in the host genome, thereby producing a new trait in the host cell. In one example, polynucleotide sequences of the present invention may be used to construct chimeric RNA/DNA oligonucleotides corresponding to said sequences, specifically designed to induce host cell mismatch repair mechanisms in an organism upon systemic injection, for example (Bartlett, R. J., et al., 2002, Nat. Biotech, 18:615-622, which is hereby incorporated by reference herein in its entirety). Such RNA/DNA oligonucleotides could be designed to correct genetic defects in certain host strains, and/or to introduce desired phenotypes in the host (e.g., introduction of a specific polymorphism within an endogenous gene corresponding to a polynucleotide of the present invention that may ameliorate and/or prevent a disease symptom and/or disorder, etc.).

Alternatively, the polynucleotide sequence of the present invention can be used to construct duplex oligonucleotides corresponding to said sequence, specifically designed to correct genetic defects in certain host strains, and/or to introduce desired phenotypes into the host (e.g., introduction of a specific polymorphism within an endogenous gene corresponding to a polynucleotide of the present invention that can ameliorate and/or prevent a disease symptom and/or disorder, etc). Such methods of using duplex oligonucleotides are known in the art and are encompassed by the present invention (see EP1007712, which is hereby incorporated by reference herein in its entirety).

### EXAMPLES

### Example 1

### Method of Genotyping Each SNP of the Present Invention

Genomic DNA samples from patients enrolled in a Bristol Myers Squibb Company clinical trial for the DPP-IV inhibitor, Saxagliptin, were genotyped for SNP1 in the human CYP3A5 and IPF1 candidate genes and evaluated relative to each patients response.

Genotyping was performed using the 5′ nuclease assay, essentially as described (Ranade K et al., 2001, Genome Research 11: 1262-1268, which is hereby incorporated by reference herein in its entirety), with the following modifications: six nanograms of genomic DNA were used in a 8 ul reaction. All PCR reactions were performed in an ABI 9700 machine and fluorescence was measured using an ABI 7900 machine.

Genotyping of the SNPs of the present invention was performed using sets of Taqman probes (100 uM each) and primers (100 uM each) specific to each SNP. Each probe/primer set was manually designed using ABI Primer Express software (Applied Biosystems). Genomic samples were prepared as described herein. The following Taqman probes and primers were utilized for one of the CYP3A5 and IPF-1 SNPs.

| | Taqman Forward | Taqman Reverse | Reference | Variable |
|---|---|---|---|---|
| SNP | Primer | Primer | Taqman Probe | Taqman Probe |
| CYP3A5 | ACCCAGCTTAACG | GAAGGGTAATGT | TGTCTTTCAG | TGTCTTTCAA |
| SNP1 | AATGCTCTACT | GGTCCAAACAG | TATCTCTTC | TATCTCTT |
| | (SEQ ID NO: 3) | (SEQ ID NO: 4) | (SEQ ID NO: 6) | (SEQ ID NO: 5) |
| IPF-1 | ACGTGACCCCCAG | CCTGAGAGCCAG | CAGCCAGACT | CAGCCGGACT |
| SNP1 | AACAATATTCCT | CAAATTCTCCAT | TCTGC | TCTG |
| | (SEQ ID NO: 13) | (SEQ ID NO: 14) | (SEQ ID NO: 15) | (SEQ ID NO: 16) |

** The allelic nucleotide in each probe sequence is shown in bold and underlined.

The genotype assay conditions are provided below.

| Components: | Final Concentration: | |
|---|---|---|
| 2x PE Master Mix (#4318157) | 1X | |
| 100uM FAM labeled probe | 200 | nmol |
| 100uM VIC labeled probe | 200 | nmol |
| Forward PCR primer | 600 | nmol |
| Reverse PCR primer | 600 | nmol |
| 6 ng template DNA | as required | |
| ddH20 | volume to 8 | ul |

Taqman thermo-cycling was performed on Perkin Elmer PE 9700 machines using the following cycling conditions below:

1) 50 C for 2 minutes

2) 95 C for 10 seconds*

3) 94 C for 15 seconds

4) 62 C for 1 minute

5) 4 C hold

Steps 2-4 were cycled 40 times

Analysis of genotypes was performed by using the Applied Biosystems ABI 7900 HT sequence detection system.

### Example 2

### Statistical Analysis of the Association Between Haart-Dependent Metabolic Abnormalities and the SNPs of the Present Invention

The association between favorable DPP-IV inhibitor therapy response and the single nucleotide polymorphisms of the present invention were investigated by applying statistical analysis to the results of the genotyping assays described herein. The central hypothesis of this analysis is that a predisposition to achieve a favorable DPP-IV inhibitor response may be conferred by specific genomic factors. The analysis attempted to identify one or more of these factors in genomic DNA samples from index cases and matched control subjects who were exposed to DPP-IV inhibitor therapy in a clinical study (see Example 1).

### Methods

Sample. Investigators in BMS clinical trials receiving DPP-IV inhibitor therapy.

Measures. Single nucleotide polymorphisms (SNPs) in human CYP3A5 and IPF-1 were genotyped on all subjects essentially as described in Example 1 herein. The SNPs that were genotyped likely represent a sample of the polymorphic variation in each gene and are not exhaustive with regard to coverage of the total genetic variation that may be present in each gene. The SNP for which a statistical association to DPP-IV inhibitor-dependent metabolic abnormalities was confirmed is provided as SNP1.

Statistical Analyses. All statistical analyses were done using SPSS version 12 (Chicago, Ill., US).

Clustering: Cluster analysis was employed to identify homogeneous sub-groups that exhibited markedly different efficacy responses to DPP-IV inhibitor therapy. Baseline glycosylated hemoglobin (HbA1c) and change in HbA1C after twelve weeks of DPP-IV inhibitor therapy for each individual were used in this analysis. Individuals with similar responses and baseline HbA1c levels were grouped together, and this process was iteratively repeated until all individuals were clustered into groups. The two-step clustering routine implemented in SPSS version 12 (Chicago, Ill., US) was used. Differences in means between clusters for HbA1c were evaluated using Kruskal-Wallis test. Genetic association between SNPs and clusters was assessed using Fisher's exact test.

Results: Three distinct subgroups of patients were observed in this trial as shown in Table 2. Two subgroups had similar mean HbA1c levels at baseline but showed pronounced differences in their responses to DPP-IV inhibitor. Whereas the non-responder group experienced little change in mean HbA1c (+0.4), the other subgroup experienced a significant reduction of 1.5 in mean HbA1c (good responder). The third subgroup (responder) had a lower mean HbA1c at baseline than either of the above groups, and consequently, experienced a modest decrease of 0.6 in mean HbA1c.

TABLE 2

| | Mean HbA1c ± SD | | |
|---|---|---|---|
| | Non-responder | Responder | Good Responder |
| Measurement | N = 27 | N = 101 | N = 70 |
| Baseline | 8.5 ± 1.1 | 7.0 ± 0.4 | 8.5 ± 0.7 |
| End of study | 8.9 ± 1.3 | 6.4 ± 0.5 | 7.0 ± 0.9 |

Differences in within-group group means were significant at P<0.01. All pairwise between-group differences in means were significant at P<0.01 at end of study. At baseline difference in means between non-responder and good responder was not significant. Other pairwise comparisons were significant at P<0.01.

Age (P=0.01), race (P<0.001) and duration of diabetes (P=0.005) were significantly associated with response as shown in Table 3.

TABLE 3

| | Non-responder | Responder | Good Responder |
|---|---|---|---|
| Variable | N = 27 | N = 101 | N = 70 |
| Age, mean years ± SD | 50.6 ± 10.3 | 55.3 ± 9.9 | 51.5 ± 9.7 |
| Race, % Hispanic | 52 | 7 | 25 |
| Duration of diabetes, | 4.1 ± 4.8 | 2.0 ± 3.4 | 1.7 ± 2.0 |
| mean years ± SD | | | |

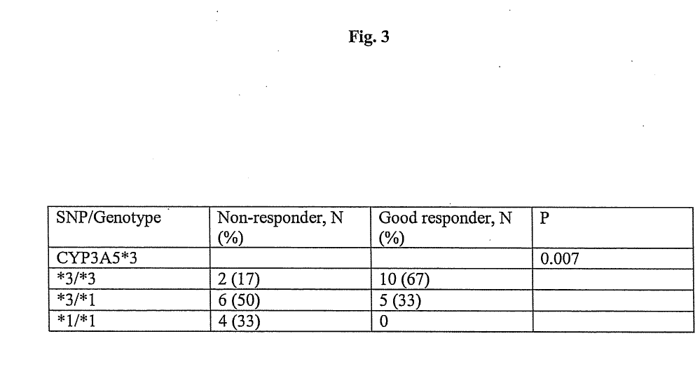

The presence of the variable allele of SNP1 of the CYP3A5 gene was shown to be significantly associated with a favorable DPP-IV inhibitor response in Hispanics (see FIG. 3). The variable allele, also referred to as the “A” allele or CYP3A5*3 allele, is known to result in missplicing of CYP3A5 mRNA by introducing a premature stop codon causing mRNA instability.

The nucleotide sequence of the CYP3A5 gene containing the reference allele (“G”) for SNP1 at nucleotide 7068 is provided in FIGS. 1A-L (SEQ ID NO:1); while the nucleotide sequence of the CYP3A5 gene containing the variable allele (“A”) for SNP1 at nucleotide 7068 is provided in FIGS. 2A-L (SEQ ID NO:2).

The nucleotide sequence of the IPF-1 gene containing the reference allele (“C”) for SNP1 at nucleotide 4445 is provided in FIGS. 4A-D (SEQ ID NO:11); while the nucleotide sequence of the IPF-1 gene containing the variable allele (“T”) for SNP1 at nucleotide 4445 is provided in FIGS. 5A-D (SEQ ID NO:12).

These results suggest that polymorphisms in the CYP3A5 and IPF-1 genes contribute to differences in the favorability of response to DPP-IV inhibitor therapy independent of other significant predictors such as age, race, and duration of diabetes.

The utility, in general, of each of these significant associations to the likelihood of achieving a favorable response to DPP-IV inhibitor therapy is that they suggest (1) such SNPs may be causally involved, alone or in combination with other SNPs, in the respective gene regions with the likelihood of achieving a favorable response to DPP-IV inhibitor therapy; (2) such SNPs, if not directly causally involved, are reflective of an association because of linkage disequilibrium with one or more other SNPs that may be causally involved, alone or in combination with other SNPs in the respective gene regions with the likelihood of achieving a favorable response to DPP-IV inhibitor therapy; (3) such SNPs may be useful in establishing haplotypes that can be used to narrow the search for and identify polymorphisms or combinations of polymorphisms that may be causally, alone or in combination with other SNPs, in the respective gene regions with the likelihood of achieving a favorable response to DPP-IV inhibitor therapy; and (4) such SNPs, if used to establish haplotypes that are identified as causally involved in such event susceptibility, can be used to predict which subjects are most likely to achieve a favorable response to DPP-IV inhibitor therapy. The term “respective gene regions” shall be construed to refer to those regions of each gene which have been used to identify the SNPs of the invention.

### Example 3

### Method of Isolating the Native Forms of the Human CYP3A5 Gene

A number of methods have been described in the art that can be utilized in isolating the native forms of the human CYP3A5 gene. Rather than describe known methods here, several specific methods are referenced below and are hereby incorporated by reference herein in their entireties. The artisan, skilled in the molecular biology arts, would be able to isolate the native form of human CYP3A5 based upon the methods and information contained, and/or referenced, therein. Quaranta, S. et al., 2006, Xenobiotica 36 (12), 1191-1200; Haufroid, V., et al., 2006, Am J Transplant 6 (11), 2706-2713; Hu, Y. F., et al., 2006, Clin. Exp. Pharmacol. Physiol. 33 (11), 1093-1098; Soars, M. G., et al., 2006, Xenobiotica 36 (4), 287-299; Dilger, K., et al., 2006, Liver Int. 26 (3), 285-290; Kuehl, P, et al., 2001, Nature Genetics, 27, pp. 383-391; Murray, G. I., et al., 1995, FEBS Lett. 364 (1), 79-82; McKinnon, R. A., et al., 1995, Gut 36 (2), 259-267; Jounaidi, Y., et al., 1994, Biochem. Biophys. Res. Commun. 205 (3), 1741-1747; Kolars, J. C., et al., 1994, Pharmacogenetics 4 (5), 247-259; T., et al., 1989, J. Biol. Chem. 264 (18), 10388-10395.

Additional methods for isolating the human CYP3A5 gene can also be found in the references cited in the Genbank accession nos. for each gene provided herein which are publically available and are also hereby incorporated by reference herein. For example, additional methods for isolating the human CYP3A5 gene can be found in the Genbank data base under the accession number NM_{—}000777 (Human CYP3A5*1 (gi|NM_{—}000777; SEQ ID NO:1; chr7:98890468-98922257 5′pad=0 3′pad=0 revComp=TRUE strand=−repeatMasking=none).

### Example 4

### Method of Isolating the Polymorphic Forms of the Human CYP3A5 Gene of the Present Invention

Since the allelic genes of the present invention represent genes present within at least a subset of the human population, these genes can be isolated using the methods provided in Example 3 above. For example, the source DNA used to isolate the allelic gene can be obtained through a random sampling of the human population and repeated until the allelic form of the gene is obtained. Preferably, random samples of source DNA from the human population are screened using the SNPs and methods of the present invention to identify those sources that comprise the allelic form of the gene. Once identified, such a source can be used to isolate the allelic form of the gene(s). The invention encompasses the isolation of such allelic genes from both genomic and/or cDNA libraries created from such source(s).

In reference to the specific methods provided in Example 3 above, it is expected that isolating the polymorphic alleles of the human CYP3A5 gene would be within the skill of an artisan trained in the molecular biology arts. Nonetheless, a detailed exemplary method of isolating at least one of the CYP3A5 polymorphic alleles, in this case the variant form of SNP1 (“A” nucleotide at 7068 of SEQ ID NO:1) is provided.

First, the individuals with the “A” allele at the locus corresponding to nucleotide 7068 of SEQ ID NO:1 or 2 are identified by genotyping the genomic DNA samples using the method outlined in Example 1 herein. Other methods of genotyping can be employed, such as the FP-SBE method (Chen et al., 1999, Genome Res., 9(5):492-498), or other methods described herein. DNA samples publicly available (e.g., from the Coriell Institute (Collingswood, N.J.) or from the clinical samples described herein can be used. Oligonucleotide primers that are used for this genotyping assay are provided in Example 2.

By analyzing genomic DNA samples, individuals with the G7068A form of the SNP1 variant can be identified. Once identified, clones comprising the genomic sequence can be obtained using methods well known in the art (see Sambrook, J., E. F. Fritsch, and T. Maniatis, 1989, Molecular Cloning: A Laboratory Manual, Cold Spring Harbor Laboratory Press, Cold Spring Harbor, N.Y.; and Current Protocols in Molecular Biology, 1995, F. M., Ausubel et al., eds, John Wiley and Sons, Inc., which are hereby incorporated by reference herein.).

If cDNA clones of the coding sequence of this allele of the gene are of interest, such clones can be obtained in accordance with the following steps. Next, Lymphoblastoid cell lines can be obtained from the Coriell Institute. These cells can be grown in RPMI-1640 medium with L-glutamine plus 10% FCS at 37 degrees. PolyA+ RNA is then isolated from these cells using Oligotex Direct Kit (Life Technologies).

First strand cDNA (complementary DNA) is produced using Superscript Preamplification System for First Strand cDNA Synthesis (Life Technologies, Cat No 18089-011) using these polyA+ RNA as templates, as specified in the users manual which is hereby incorporated herein by reference in its entirety. Specific cDNA encoding the human CYP3A5 protein is amplified by polymerase chain reaction (PCR) using a forward primer which hybridizes to the 5′-UTR region, a reverse primer which hybridizes to the 3′-UTR region, and these first strand cDNA as templates (Sambrook et al., 1989, Id.). Alternatively, these primers can be designed using Primer3 program (Rozen et al, 2000, pp. 365-386, Bioinformatics Methods and Protocols in Methods of Molecular Biology, S. Krawetz, S. Misener, Eds., Humana Press, Totowa, N.J.). Restriction enzyme sites (example: SalI for the forward primer, and NotI for reverse primer) are added to the 5′-end of these primer sequences to facilitate cloning into expression vectors after PCR amplification. PCR amplification can be performed essentially as described in the owner's manual of the Expand Long Template PCR System (Roche Molecular Biochemicals) following manufacturer's standard protocol, which is hereby incorporated herein by reference in its entirety.

PCR amplification products are digested with restriction enzymes (such as SalI and NotI, for example) and ligated with expression vector DNA cut with the same set of restriction enzymes. pSPORT (Invitrogen) is one example of such an expression vector. After ligated DNA is introduced into E. coli cells (Sambrook, et al. 1989, Id.), plasmid DNA is isolated from these bacterial cells. This plasmid DNA is sequenced to confirm the presence an intact (full-length) coding region of the human CYP3A5 protein with the variation, if the variation results in changes in the encoded amino acid sequence, using methods well known in the art and described elsewhere herein.

The skilled artisan would appreciate that the above method can be applied to isolating the other novel human CYP3A5 genes of the present invention through the simple substitution of applicable PCR and sequencing primers. Such primers can be selected from any one of the applicable primers provided in herein, or can be designed using the Primer3 program program (Rozen S, et al., 2000, Id.) as described. Such primers can preferably comprise at least a portion of any one of the polynucleotide sequences of the present invention.

### Example 5

### Method of Engineering the Allelic Forms of the Human CYP3A5 Gene of the Present Invention

Aside from isolating the allelic genes of the present invention from DNA samples obtained from the human population, as described in Example 4 above, the invention also encompasses methods of engineering the allelic genes of the present invention through the application of site-directed mutagenesis to the isolated native forms of the genes. Such methodology could be applied to synthesize allelic forms of the genes comprising at least one, or more, of the encoding SNPs of the present invention (e.g., silent, missense)—preferably at least 1, 2, 3, or 4 encoding SNPs for each gene.

In reference to the specific methods provided in Example 4 above, it is expected that isolating the novel polymorphic CYP3A5 genes of the present invention would be within the ordinary skill of an artisan trained in the molecular biology arts. Nonetheless, a detailed exemplary method of engineering at least one of the CYP3A5 polymorphic alleles to comprise the encoding and/or non-coding polymorphic nucleic acid sequence, in this case the variant form (G7068A) of SNP1 (SEQ ID NO:2) is provided. Briefly, genomic clones containing the human CYP3A5 gene can be identified by homology searches with the BLASTN program (Altschul, S F et al., 1990, J. Mol. Biol. 215: 403-410) against the Genbank non-redundant nucleotide sequence database using the published human CYP3A5 cDNA sequence (GenBank Accession No.: NM_{—}000777). Alternatively, the genomic sequence of the human CYP3A5 gene can be obtained as described herein. After obtaining these clones, they are sequenced to confirm the validity of the DNA sequences.

However, in the case of the variant form (G7068A) of SNP1, genomic clones would need to be obtained and can be identified by homology searches with the BLASTN program (Altschul SF, 1990, Id.) against the Genbank non-redundant nucleotide sequence database using the published human CYP3A5 genomic sequence (GenBank Accession No.: NM_{—}000777). Alternatively, the genomic sequence of the human CYP3A5 gene can be obtained as described herein. After obtaining these clones, they are sequenced to confirm the validity of the DNA sequences.

Once these clones are confirmed to contain the intact wild type cDNA or genomic sequence of the human CYP3A5 coding and/or non-coding region, the G7068A polymorphism (mutation) can be introduced into the native sequence using PCR directed in vitro mutagenesis (Cormack, B., Directed Mutagenesis Using the Polymerase Chain Reaction. Current Protocols in Molecular Biology, John Wiley & Sons, Inc. Supplement 37: 8.5.1-8.5.10, (2000)). In this method, synthetic oligonucleotides are designed to incorporate a point mutation at one end of an amplified fragment. Following polymerase chain reaction (PCR), the amplified fragments are made blunt-ended by treatment with Klenow Fragment. These fragments are then ligated and subcloned into a vector to facilitate sequence analysis. This method consists of the following steps.

1. Subcloning of cDNA or genomic insert into a plasmid vector, or BAC sequence if the clone is a genomic sequence, containing multiple cloning sites and M13 flanking sequences, such as pUC19 (Sambrook et al., 1989, Id.), in the forward orientation. The skilled artisan would appreciate that other plasmids could be equally substituted, and may be desirable in certain circumstances.

2. Introduction of a mutation by PCR amplification of the genomic region downstream of the mutation site using a primer including the mutation. (FIG. 8.5.2 in Cormack, 2000, Id.)). In the case of introducing the G7068A mutation into the human CYP3A5 genomic sequence, the following two primers can be used.

M13 reverse sequencing primer:
(SEQ ID NO: 7)
5′-AGCGGATAACAATTTCACACAGGA-3′.
Mutation primer:
(SEQ ID NO: 8)
5′-GAGCTCTTTTGTCTTTCAATATCTCTTCCCTGTTTGGAC-3′

Mutation primer contains the mutation (G7068A) at the 5′ end (in bold and underlined) and a portion of its flanking sequence. M13 reverse sequencing primer hybridizes to the pUC19 vector. Subcloned cDNA or genomic clone comprising the human CYP3A5 cDNA or genomic sequence is used as a template (described in Step 1). A 100 ul PCR reaction mixture is prepared using 10 ng of the template DNA, 200 uM 4dNTPs, 1 uM primers, 0.25U Taq DNA polymerase (PE), and standard Taq DNA polymerase buffer. Typical PCR cycling condition are as follows:

20-25 cycles: 45 sec, 93 degrees
2 min, 50 degrees
2 min, 72 degrees

1 cycle: 10 min, 72 degrees

After the final extension step of PCR, 5U Klenow Fragment is added and incubated for 15 minutes at 30 degrees. The PCR product is then digested with the restriction enzyme, EcoRI.

3. PCR amplification of the upstream region is then performed, using subcloned cDNA or genomic clone as a template (the product of Step 1). This PCR is done using the following two primers:

M13 forward sequencing primer:
(SEQ ID NO: 9)
5′-CGCCAGGGTTTTCCCAGTCACGAC-3′.
Flanking primer:
(SEQ ID NO: 10)
5′-GTCCAAACAGGGAAGAGATATTGAAAGACAAAAGAGCTC-3′.

Flanking primer is complementary to the upstream flanking sequence and mutation locus of the G7068A mutation (in bold and underlined). M13 forward sequencing primer hybridizes to the pUC19 vector. PCR conditions and Klenow treatments follow the same procedures as provided in Step 2, above. The PCR product is then digested with the restriction enzyme, HindIII.

4. Prepare the pUC19 vector for cloning the cDNA or genomic clone comprising the polymorphic locus. Digest pUC19 plasmid DNA with EcoRI and HindIII. The resulting digested vector fragment can then be purified using techniques well known in the art, such as gel purification, for example.

5. Combine the products from Step 2 (PCR product containing mutation), Step 3 (PCR product containing the upstream region), and Step 4 (digested vector), and ligate them together using standard blunt-end ligation conditions (Sambrook, et al., 1989. Id.).

6. Transform the resulting recombinant plasmid from Step 5 into E. coli competent cells using methods known in the art, such as, for example, the transformation methods described in Sambrook, et al., 1989, Id.

7. Analyze the amplified fragment portion of the plasmid DNA by DNA sequencing to confirm the point mutation, and absence of any other mutations introduced during PCR. The method of sequencing the insert DNA, including the primers utilized, are described herein or are otherwise known in the art.

### Example 6

### Method of Isolating the Native Forms of the Human IPF-1 Gene

A number of methods have been described in the art that can be utilized in isolating the native forms of the human IPF-1 gene. Rather than describe known methods here, several specific methods are referenced below and are hereby incorporated by reference herein in their entireties. The artisan, skilled in the molecular biology arts, would be able to isolate the native form of human IPF-1 based upon the methods and information contained, and/or referenced, therein. Liu, A., et al., FEBS Lett. 580 (28-29), 6701-6706 (2006); Elbein, S. C., et al., Diabetes 55 (10), 2909-2914 (2006); Maedler, K., et al., Diabetes 55 (9), 2455-2462 (2006); Malecki, M. T., et al., Diabetologia 49 (8), 1985-1987 (2006); Lin, H. T., et al., World J. Gastroenterol. 12 (28), 4529-4535 (2006); Marshak, S., et al., Proc. Natl. Acad. Sci. U.S.A. 93 (26), 15057-15062 (1996); Watada, H., et al., Biochem. Biophys. Res. Commun. 229 (3), 746-751 (1996); Waeber, G., et al., Mol. Endocrinol. 10 (11), 1327-1334 (1996); Stoffel, M., et al., Genomics 28 (1), 125-126 (1995); Leonard, J., et al., Mol. Endocrinol. 7 (10), 1275-1283 (1993).

Additional methods for isolating the human IPF-1 gene of the present invention can also be found in the references cited in the Genbank accession nos. for each gene provided herein which are publically available and are also hereby incorporated by reference herein. For example, additional methods for isolating the human IPF-1 gene can be found in the Genbank data base under the accession number NM_{—}000209 Human IPF1 (gi|NM_{—}000209; SEQ ID NO:11; range=chr13:27391177-27398394 (from Human Genome Gateway Browser)).

### Example 7

### Method of Isolating the Polymorphic Forms of the Human IPF-1 Gene of the Present Invention

Since the allelic genes of the present invention represent genes present within at least a subset of the human population, these genes can be isolated using the methods provided in Example 6 above. For example, the source DNA used to isolate the allelic gene can be obtained through a random sampling of the human population and repeated until the allelic form of the gene is obtained. Preferably, random samples of source DNA from the human population are screened using the SNPs and methods of the present invention to identify those sources that comprise the allelic form of the gene. Once identified, such a source can be used to isolate the allelic form of the gene(s). The invention encompasses the isolation of such allelic genes from both genomic and/or cDNA libraries created from such source(s).

In reference to the specific methods provided in Example 6 above, it is expected that isolating the polymorphic alleles of the human IPF-1 gene would be within the skill of an artisan trained in the molecular biology arts. Nonetheless, a detailed exemplary method of isolating at least one of the IPF-1 polymorphic alleles, in this case the variant form of SNP1 (“T” nucleotide at 4445 of SEQ ID NO:11) is provided.

First, the individuals with the “T” allele at the locus corresponding to nucleotide 4445 of SEQ ID NO:11 or 12 are identified by genotyping the genomic DNA samples using the method outlined in Example 1 herein. Other methods of genotyping may be employed, such as the FP-SBE method (Chen et al., Genome Res., 9(5):492-498 (1999)), or other methods described herein. DNA samples publicly available (e.g., from the Coriell Institute (Collingswood, N.J.)) or from the clinical samples described herein may be used. Oligonucleotide primers that are used for this genotyping assay are provided in Example 1.

By analyzing genomic DNA samples, individuals with the C4445T form of the SNP1 variant can be identified. Once identified, clones comprising the genomic sequence can be obtained using methods well known in the art (see Sambrook, J., E. F. Fritsch, and T. Maniatis, 1989, Molecular Cloning: A Laboratory Manual, Cold Spring Harbor Laboratory Press, Cold Spring Harbor, N.Y.; and Current Protocols in Molecular Biology, 1995, F. M., Ausubel et al., eds, John Wiley and Sons, Inc., which are hereby incorporated by reference herein.).

If cDNA clones of the coding sequence of this allele of the gene are of interest, such clones can be obtained in accordance with the following steps. Next, Lymphoblastoid cell lines can be obtained from the Coriell Institute. These cells can be grown in RPMI-1640 medium with L-glutamine plus 10% FCS at 37 degrees. PolyA+ RNA is then isolated from these cells using Oligotex Direct Kit (Life Technologies).

First strand cDNA (complementary DNA) is produced using Superscript Preamplification System for First Strand cDNA Synthesis (Life Technologies, Cat No 18089-011) using these polyA+ RNA as templates, as specified in the users manual which is hereby incorporated herein by reference in its entirety. Specific cDNA encoding the human IPF-1 protein is amplified by polymerase chain reaction (PCR) using a forward primer which hybridizes to the 5′-UTR region, a reverse primer which hybridizes to the 3′-UTR region, and these first strand cDNA as templates (Sambrook, et al., 1989, Id.). Alternatively, these primers may be designed using Primer3 program (Rozen S, 2000, Id.). Restriction enzyme sites (example: SalI for the forward primer, and NotI for reverse primer) are added to the 5′-end of these primer sequences to facilitate cloning into expression vectors after PCR amplification. PCR amplification may be performed essentially as described in the owner's manual of the Expand Long Template PCR System (Roche Molecular Biochemicals) following manufacturer's standard protocol, which is hereby incorporated herein by reference in its entirety.

PCR amplification products are digested with restriction enzymes (such as SalI and NotI, for example) and ligated with expression vector DNA cut with the same set of restriction enzymes. pSPORT (Invitrogen) is one example of such an expression vector. After ligated DNA is introduced into E. coli cells (Sambrook, Fritsch et al. 1989), plasmid DNA is isolated from these bacterial cells. This plasmid DNA is sequenced to confirm the presence an intact (full-length) coding region of the human IPF-1 protein with the variation, if the variation results in changes in the encoded amino acid sequence, using methods well known in the art and described elsewhere herein.

The skilled artisan would appreciate that the above method can be applied to isolating the other novel human IPF-1 genes of the present invention through the simple substitution of applicable PCR and sequencing primers. Such primers can be selected from any one of the applicable primers provided in herein, or can be designed using the Primer3 program (Rozen S, 2000, Id.) as described. Such primers can preferably comprise at least a portion of any one of the polynucleotide sequences of the present invention.

### Example 8

### Method of Engineering the Allelic Forms of the Human IPF-1 Gene of the Present Invention

Aside from isolating the allelic genes of the present invention from DNA samples obtained from the human population, as described in Examples 6 and 7 above, the invention also encompasses methods of engineering the allelic genes of the present invention through the application of site-directed mutagenesis to the isolated native forms of the genes. Such methodology could be applied to synthesize allelic forms of the genes comprising at least one, or more, of the encoding SNPs of the present invention (e.g., silent, missense)—preferably at least 1, 2, 3, or 4 encoding SNPs for each gene.

In reference to the specific methods provided in Example 6 and 7 above, it is expected that isolating the novel polymorphic IPF-1 genes of the present invention would be within the skill of an artisan trained in the molecular biology arts. Nonetheless, a detailed exemplary method of engineering at least one of the IPF-1 polymorphic alleles to comprise the encoding and/or non-coding polymorphic nucleic acid sequence, in this case the variant form (C4445T) of SNP1 (SEQ ID NO:12) is provided. Briefly, genomic clone containing the human IPF-1 gene may be identified by homology searches with the BLASTN program (Altschul S F, 1990, Id.) against the Genbank non-redundant nucleotide sequence database using the published human IPF-1 cDNA sequence (GenBank Accession No.: NM_{—}000209). Alternatively, the genomic sequence of the human IPF-1 gene may be obtained as described herein. After obtaining these clones, they are sequenced to confirm the validity of the DNA sequences.

However, in the case of the variant form (C4445T) of SNP1, genomic clones would need to be obtained and can be identified by homology searches with the BLASTN program (Altschul S F, 1990, Id.) against the Genbank non-redundant nucleotide sequence database using the published human IPF1 genomic sequence (GenBank Accession No.: NM_{—}000209). Alternatively, the genomic sequence of the human IPF-1 gene may be obtained as described herein. After obtaining these clones, they are sequenced to confirm the validity of the DNA sequences.

Once these clones are confirmed to contain the intact wild type cDNA or genomic sequence of the human IPF-1 coding and/or non-coding region, the C4445T polymorphism (mutation) may be introduced into the native sequence using PCR directed in vitro mutagenesis (Cormack, B., Directed Mutagenesis Using the Polymerase Chain Reaction. Current Protocols in Molecular Biology, John Wiley & Sons, Inc. Supplement 37: 8.5.1-8.5.10, (2000)). In this method, synthetic oligonucleotides are designed to incorporate a point mutation at one end of an amplified fragment. Following PCR, the amplified fragments are made blunt-ended by treatment with Klenow Fragment. These fragments are then ligated and subcloned into a vector to facilitate sequence analysis. This method consists of the following steps.

1. Subcloning of cDNA or genomic insert into a plasmid vector, or BAC sequence if the clone is a genomic sequence, containing multiple cloning sites and M13 flanking sequences, such as pUC19 (Sambrook, et al. 1989, Id.), in the forward orientation. The skilled artisan would appreciate that other plasmids could be equally substituted, and may be desirable in certain circumstances.

2. Introduction of a mutation by PCR amplification of the genomic region downstream of the mutation site using a primer including the mutation. (FIG. 8.5.2 in Cormack, 2000, Id.)). In the case of introducing the C4445T mutation into the human IPF-1 genomic sequence, the following two primers may be used.

M13 reverse sequencing primer:
(SEQ ID NO: 7)
5′-AGCGGATAACAATTTCACACAGGA-3′.
Mutation primer:
(SEQ ID NO: 17)
5′-CTTCACTTCGCGGGCAGAAGTCTGGCTGAAGTTAAAACAATTATG-3′

Mutation primer contains the mutation (C4445T) at the 5′ end (in bold and underlined) and a portion of its flanking sequence. M13 reverse sequencing primer hybridizes to the pUC19 vector. Subcloned cDNA or genomic clone comprising the human IPF-1 cDNA or genomic sequence is used as a template (described in Step 1). A 100 ul PCR reaction mixture is prepared using 10 ng of the template DNA, 200 uM 4dNTPs, 1 uM primers, 0.25U Taq DNA polymerase (PE), and standard Taq DNA polymerase buffer. Typical PCR cycling condition are as follows:

20-25 cycles: 45 sec, 93 degrees
2 min, 50 degrees
2 min, 72 degrees

1 cycle: 10 min, 72 degrees

After the final extension step of PCR, 5U Klenow Fragment is added and incubated for 15 minutes at 30 degrees. The PCR product is then digested with the restriction enzyme, EcoRI.

3. PCR amplification of the upstream region is then performed, using subcloned cDNA or genomic clone as a template (the product of Step 1). This PCR is done using the following two primers:

M13 forward sequencing primer:
(SEQ ID NO: 9)
5′-CGCCAGGGTTTTCCCAGTCACGAC-3′.
Flanking primer:
(SEQ ID NO: 18)
5′-CATAATTGTTTTAACTTCAGCCAGACTTCTGCCCGCGAAGTGA
AG-3′.

Flanking primer is complementary to the upstream flanking sequence and mutation locus of the C4445T mutation (in bold and underlined). M13 forward sequencing primer hybridizes to the pUC19 vector. PCR conditions and Klenow treatments follow the same procedures as provided in Step 2, above. The PCR product is then digested with the restriction enzyme, HindIII.

4. Prepare the pUC19 vector for cloning the cDNA or genomic clone comprising the polymorphic locus. Digest pUC19 plasmid DNA with EcoRI and HindIII. The resulting digested vector fragment may then be purified using techniques well known in the art, such as gel purification, for example.

5. Combine the products from Step 2 (PCR product containing mutation), Step 3 (PCR product containing the upstream region), and Step 4 (digested vector), and ligate them together using standard blunt-end ligation conditions (Sambrook, et al. 1989, Id.).

6. Transform the resulting recombinant plasmid from Step 5 into E. coli competent cells using methods known in the art, such as, for example, the transformation methods described in Sambrook, et al., 1989, Id.

7. Analyze the amplified fragment portion of the plasmid DNA by DNA sequencing to confirm the point mutation, and the absence of any other mutations introduced during PCR. The method of sequencing the insert DNA, including the primers utilized, are described herein or are otherwise known in the art.

### Example 9

### Alternative Methods of Genotyping Polymorphisms Encompassed by the Present Invention

### Preparation of Samples

Polymorphisms are detected in a target nucleic acid from an individual being analyzed. For assay of genomic DNA, virtually any biological sample (other than pure red blood cells) is suitable. For example, convenient tissue samples include whole blood, semen, saliva, tears, urine, fecal material, sweat, buccal, skin and hair. For assay of cDNA or mRNA, the tissue sample must be obtained from an organ in which the target nucleic acid is expressed. For example, if the target nucleic acid is a cytochrome P450, the liver is a suitable source.

Many of the methods described below require amplification of DNA from target samples. This can be accomplished, for example, by polymerase chain reaction (PCR). See generally PCR Technology: Principles and Applications for DNA Amplification (ed. H. A. Erlich, Freeman Press, NY, N.Y., 1992); PCR Protocols: A Guide to Methods and Applications (eds. Innis, et al., Academic Press, San Diego, Calif., 1990); Mattila et al., Nucleic Acids Res. 19, 4967 (1991); Eckert et al., PCR Methods and Applications 1, (1991); PCR (eds. McPherson et al., IRL Press, Oxford); and U.S. Pat. No. 4,683,202.

Other suitable amplification methods include the ligase chain reaction (LCR) (see Wu and Wallace, 1989, Genomics 4:560; Landegren et al., 1988, Science 241:1077; transcription amplification (Kwoh et al., 1989, Proc. Natl. Acad. Sci. USA 86, 1173; self-sustained sequence replication (Guatelli et al., 1990, Proc. Nat. Acad. Sci. USA, 87:1874) and nucleic acid based sequence amplification (NASBA). The latter two amplification methods involve isothermal reactions based on isothermal transcription, which produce both single stranded RNA (ssRNA) and double stranded DNA (dsDNA) as the amplification products in a ratio of about 30 or 100 to 1, respectively. Additional methods of amplification are known in the art or are described elsewhere herein.

### Detection of Polymorphisms in Target DNA

There are two distinct types of analysis of target DNA for detecting polymorphisms. The first type of analysis, sometimes referred to as de novo characterization, is carried out to identify polymorphic sites not previously characterized (i.e., to identify new polymorphisms). This analysis compares target sequences in different individuals to identify points of variation, i.e., polymorphic sites. By analyzing groups of individuals representing the greatest ethnic diversity among humans and greatest breed and species variety in plants and animals, patterns characteristic of the most common alleles/haplotypes of the locus can be identified, and the frequencies of such alleles/haplotypes in the population can be determined. Additional allelic frequencies can be determined for subpopulations characterized by criteria such as geography, race, or gender. The de novo identification of polymorphisms of the invention is described in the Examples section.

The second type of analysis determines which form(s) of a characterized (known) polymorphism are present in individuals under test. Additional methods of analysis are known in the art or are described elsewhere herein.

### Allele-Specific Probes

The design and use of allele-specific probes for analyzing polymorphisms is described, for example, by Saiki et al., 1986, Nature 324, 163-166; Dattagupta, EP 235,726, and Saiki, WO 89/11548. Allele-specific probes can be designed that hybridize to a segment of target DNA from one individual but do not hybridize to the corresponding segment from another individual due to the presence of different polymorphic forms in the respective segments from the two individuals. Hybridization conditions should be sufficiently stringent so that there is a significant difference in hybridization intensity between alleles, and preferably an essentially binary response, whereby a probe hybridizes to only one of the alleles. Some probes are designed to hybridize to a segment of target DNA such that the polymorphic locus aligns with a central position (e.g., in a 15-mer at the 7 position; in a 16-mer, at either the 8 or 9 position) of the probe. This design of probe achieves good discrimination in hybridization between different allelic forms.

Allele-specific probes are often used in pairs, one member of a pair showing a perfect match to a reference form of a target sequence and the other member showing a perfect match to a variant form. Several pairs of probes can then be immobilized on the same support for simultaneous analysis of multiple polymorphisms within the same target sequence.

### Tiling Arrays

The polymorphisms can also be identified by hybridization to nucleic acid arrays, some examples of which are described in WO 95/11995. The same arrays or different arrays can be used for analysis of characterized polymorphisms. WO 95/11995 also describes sub arrays that are optimized for detection of a variant form of a precharacterized polymorphism. Such a sub array contains probes designed to be complementary to a second reference sequence, which is an allelic variant of the first reference sequence. The second group of probes is designed by the same principles as described, except that the probes exhibit complementarity to the second reference sequence. The inclusion of a second group (or additional groups) can be particularly useful for analyzing short subsequences of the primary reference sequence in which multiple mutations are expected to occur within a short distance commensurate with the length of the probes (e.g., two or more mutations within 9 bases).

### Allele-Specific Primers

An allele-specific primer hybridizes to a site on target DNA overlapping a polymorphism and only primes amplification of an allelic form to which the primer exhibits perfect complementarity. See Gibbs, Nucleic Acid Res. 17, 2427-2448 (1989). This primer is used in conjunction with a second primer which hybridizes at a distal site. Amplification proceeds from the two primers, resulting in a detectable product which indicates the particular allelic form is present. A control is usually performed with a second pair of primers, one of which shows a single base mismatch at the polymorphic locus and the other of which exhibits perfect complementarity to a distal site. The single-base mismatch prevents amplification and no detectable product is formed. The method works best when the mismatch is included in the 3′-most position of the oligonucleotide aligned with the polymorphism because this position is the most destabilizing elongation from the primer (see, e.g., WO 93/22456).

### Direct-Sequencing

The direct analysis of the sequence of polymorphisms of the present invention can be accomplished using either the dideoxy chain termination method or the Maxam—Gilbert method (see Sambrook et al., Molecular Cloning, A Laboratory Manual (2nd Ed., CSHP, New York 1989); Zyskind et al., Recombinant DNA Laboratory Manual, (Acad. Press, 1988)).

### Denaturing Gradient Gel Electrophoresis

Amplification products generated using the polymerase chain reaction can be analyzed by the use of denaturing gradient gel electrophoresis. Different alleles can be identified based on the different sequence-dependent melting properties and electrophoretic migration of DNA in solution. Erlich, ed., PCR Technology. Principles and Applications for DNA Amplification, (W. H. Freeman and Co, New York, 1992), Chapter 7.

### Single-Strand Conformation Polymorphism Analysis

Alleles of target sequences can be differentiated using single-strand conformation polymorphism analysis, which identifies base differences by alteration in electrophoretic migration of single stranded PCR products, as described in Orita et al., Proc. Nat. Acad. Sci. 86, 2766-2770 (1989). Amplified PCR products can be generated as described above, and heated or otherwise denatured, to form single stranded amplification products. Single-stranded nucleic acids may refold or form secondary structures which are partially dependent on the base sequence. The different electrophoretic mobilities of single-stranded amplification products can be related to base-sequence differences between alleles of target sequences.

### Single Base Extension

An alternative method for identifying and analyzing polymorphisms is based on single-base extension (SBE) of a fluorescently-labeled primer coupled with fluorescence resonance energy transfer (FRET) between the label of the added base and the label of the primer. Typically, the method, such as that described by Chen et al., 1997, PNAS 94:10756-61, uses a locus-specific oligonucleotide primer labeled on the 5′ terminus with 5-carboxyfluorescein (F AM). This labeled primer is designed so that the 3′ end is immediately adjacent to the polymorphic locus of interest. The labeled primer is hybridized to the locus, and single base extension of the labeled primer is performed with fluorescently-labeled dideoxyribonucleotides (ddNTPs) in dye-terminator sequencing fashion. An increase in fluorescence of the added ddNTP in response to excitation at the wavelength of the labeled primer is used to infer the identity of the added nucleotide.

### Example 10

### Additional methods of genotyping the SNPs of the Present Invention

The skilled artisan would acknowledge that there are a number of methods that may be employed for genotyping a SNP of the present invention, aside from the preferred methods described herein. The present invention encompasses the following non-limiting types of genotype assays: PCR-free genotyping methods, Single-step homogeneous methods, Homogeneous detection with fluorescence polarization, Pyrosequencing, “Tag” based DNA chip system, Bead-based methods, fluorescent dye chemistry, Mass spectrometry based genotyping assays, TaqMan genotype assays, Invader genotype assays, and microfluidic genotype assays, among others.

Specifically encompassed by the present invention are the following, non-limiting genotyping methods: Landegren, U., Nilsson, M. & Kwok, P. Genome Res 8, 769-776 (1998); Kwok, P., Pharmacogenomics 1, 95-100 (2000); Gut, I., Hum Mutat 17, 475-492 (2001); Whitcombe, D., Newton, C. & Little, S., Curr Opin Biotechnol 9, 602-608 (1998); Tillib, S. & Mirzabekov, A., Curr Opin Biotechnol 12, 53-58 (2001); Winzeler, E. et al., Science 281, 1194-1197 (1998); Lyamichev, V. et al., Nat Biotechnol 17, 292-296 (1999); Hall, J. et al., Proc Natl Acad Sci USA 97, 8272-8277 (2000); Mein, C. et al., Genome Res 10, 333-343 (2000); Ohnishi, Y. et al., J Hum Genet. 46, 471-477 (2001); Nilsson, M. et al., Science 265, 2085-2088 (1994); Baner, J., Nilsson, M., Mendel-Hartvig, M. & Landegren, U., Nucleic Acids Res 26, 5073-5078 (1998); Baner, J. et al., Curr Opin Biotechnol 12, 11-15 (2001); Hatch, A., Sano, T., Misasi, J. & Smith, C., Genet Anal 15, 35-40 (1999); Lizardi, P. et al., Nat Genet. 19, 225-232 (1998); Zhong, X., Lizardi, P., Huang, X., Bray-Ward, P. & Ward, D., Proc Natl Acad Sci USA 98, 3940-3945 (2001); Faruqi, F. et al. BMC Genomics 2, 4 (2001); Livak, K., Genet Anal 14, 143-149 (1999); Marras, S., Kramer, F. & Tyagi, S., Genet Anal 14, 151-156 (1999); Ranade, K. et al., Genome Res 11, 1262-1268 (2001); Myakishev, M., Khripin, Y., Hu, S. & Hamer, D., Genome Re 11, 163-169 (2001); Beaudet, L., Bedard, J., Breton, B., Mercuri, R. & Budarf, M., Genome Res 11, 600-608 (2001); Chen, X., Levine, L. & PY, K., Genome Res 9, 492-498 (1999); Gibson, N. et al., Clin Chem 43, 1336-1341 (1997); Latif, S., Bauer-Sardina, I., Ranade, K., Livak, K. & P Y, K., Genome Res 11, 436-440 (2001); Hsu, T., Law, S., Duan, S., Neri, B. & Kwok, P., Clin Chem 47, 1373-1377 (2001); Alderborn, A., Kristofferson, A. & Hammerling, U., Genome Res 10, 1249-1258 (2000); Ronaghi, M., Uhlen, M. & Nyren, P., Science 281, 363, 365 (1998); Ronaghi, M., Genome Res 11, 3-11 (2001); Pease, A. et al., Proc Natl Acad Sci USA 91, 5022-5026 (1994); Southern, E., Maskos, U. & Elder, J., Genomics 13, 1008-1017 (1993); Wang, D. et al., Science 280, 1077-1082 (1998); Brown, P. & Botstein, D., Nat Genet. 21, 33-37 (1999); Cargill, M. et al. Nat Genet. 22, 231-238 (1999); Dong, S. et al., Genome Res 11, 1418-1424 (2001); Halushka, M. et al., Nat Genet. 22, 239-247 (1999); Hacia, J., Nat Genet. 21, 42-47 (1999); Lipshutz, R., Fodor, S., Gingeras, T. & Lockhart, D., Nat Genet. 21, 20-24 (1999); Sapolsky, R. et al., Genet Anal 14, 187-192 (1999); Tsuchihashi, Z. & Brown, P., J Virol 68, 5863 (1994); Herschlag, D., J Biol Chem 270, 20871-20874 (1995); Head, S. et al., Nucleic Acids Res 25, 5065-5071 (1997); Nikiforov, T. et al., Nucleic Acids Res 22, 4167-4175 (1994); Syvanen, A. et al., Genomics 12, 590-595 (1992); Shumaker, J., Metspalu, A. & Caskey, C., Hum Mutat 7, 346-354 (1996); Lindroos, K., Liljedahl, U., Raitio, M. & Syvanen, A., Nucleic Acids Res 29, E69-9 (2001); Lindblad-Toh, K. et al., Nat Genet. 24, 381-386 (2000); Pastinen, T. et al., Genome Res 10, 1031-1042 (2000); Fan, J. et al., Genome Res 10, 853-860 (2000); Hirschhorn, J. et al., Proc Natl Acad Sci USA 97, 12164-12169 (2000); Bouchie, A., Nat Biotechnol 19, 704 (2001); Hensel, M. et al., Science 269, 400-403 (1995); Shoemaker, D., Lashkari, D., Morris, D., Mittmann, M. & Davis, R. Nat Genet. 14, 450-456 (1996); Gerry, N. et al., J Mol Biol 292, 251-262 (1999); Ladner, D. et al., Lab Invest 81, 1079-1086 (2001); Iannone, M. et al., Cytometry 39, 131-140 (2000); Fulton, R., McDade, R., Smith, P., Kienker, L. & Kettman, J. J., Clin Chem 43, 1749-1756 (1997); Armstrong, B., Stewart, M. & Mazumder, A., Cytometry 40, 102-108 (2000); Cai, H. et al., Genomics 69, 395 (2000); Chen, J. et al., Genome Res 10, 549-557 (2000); Ye, F. et al. Hum Mutat 17, 305-316 (2001); Michael, K., Taylor, L., Schultz, S. & Walt, D., Anal Chem 70, 1242-1248 (1998); Steemers, F., Ferguson, J. & Walt, D., Nat Biotechnol 18, 91-94 (2000); Chan, W. & Nie, S., Science 281, 2016-2018 (1998); Han, M., Gao, X., Su, J. & Nie, S., Nat Biotechnol 19, 631-635 (2001); Griffin, T. & Smith, L., Trends Biotechnol 18, 77-84 (2000); Jackson, P., Scholl, P. & Groopman, J., Mol Med Today 6, 271-276 (2000); Haff, L. & Smirnov, I., Genome Res 7, 378-388 (1997); Ross, P., Hall, L., Smirnov, I. & Haff, L., Nat Biotechnol 16, 1347-1351 (1998); Bray, M., Boerwinkle, E. & Doris, P. Hum Mutat 17, 296-304 (2001); Sauer, S. et al., Nucleic Acids Res 28, E13 (2000); Sauer, S. et al., Nucleic Acids Res 28, E100 (2000); Sun, X., Ding, H., Hung, K. & Guo, B., Nucleic Acids Res 28, E68 (2000); Tang, K. et al., Proc Natl Acad Sci USA 91, 10016-10020 (1999); Li, J. et al., Electrophoresis 20, 1258-1265 (1999); Little, D., Braun, A., O'Donnell, M. & Koster, H., Nat Med 3, 1413-1416 (1997); Little, D. et al. Anal Chem 69, 4540-4546 (1997); Griffin, T., Tang, W. & Smith, L., Nat Biotechnol 15, 1368-1372 (1997); Ross, P., Lee, K. & Belgrader, P., Anal Chem 69, 4197-4202 (1997); Jiang-Baucom, P., Girard, J., Butler, J. & Belgrader, P., Anal Chem 69, 4894-4898 (1997); Griffin, T., Hall, J., Prudent, J. & Smith, L., Proc Natl Acad Sci USA 96, 6301-6306 (1999); Kokoris, M. et al., Mol Diagn 5, 329-340 (2000); Jurinke, C., van den Boom, D., Cantor, C. & Koster, H. (2001); and/or Taranenko, N. et al., Genet Anal 13, 87-94 (1996).

In addition, the genotyping methods described and/or claimed in U.S. Pat. No. 6,458,540 and the methods described and/or claimed in U.S. Pat. No. 6,440,707 are also encompassed by the present invention.

The entire disclosure of each document cited (including patents, patent applications, journal articles, abstracts, laboratory manuals, books, or other disclosures) in the Background of the Invention, Detailed Description, and Examples is hereby incorporated herein by reference. Further, the hard copy of the Sequence Listing submitted herewith and the corresponding computer readable form are both incorporated herein by reference in their entireties.

While this invention has been particularly shown and described with references to preferred embodiments thereof, it will be understood by those skilled in the art that various changes in form and details may be made therein without departing from the scope of the invention encompassed by the appended claims.

It will be clear that the invention may be practiced otherwise than as particularly described in the foregoing description and examples. Numerous modifications and variations of the present invention are possible in light of the above teachings and, therefore, are within the scope of the appended claims.

What is claimed is:

1. A method of identifying an individual having an increased likelihood of achieving a favorable response to the administration of a pharmaceutically acceptable amount of a DPP-IV inhibitor comprising the step of determining whether said individual has a reference or variant allele at one or more polymorphic loci of the human CYP3A5 gene, wherein the presence of a reference allele at said one or more polymorphic loci indicates a decreased likelihood of achieving a favorable response to a DPP-IV inhibitor relative to an individual harboring the variant allele at that locus.

2. A method of identifying an individual having an increased likelihood of achieving a favorable response to the administration of a pharmaceutically acceptable amount of a DPP-IV inhibitor comprising the step of determining whether said individual has a reference or variant allele at one or more polymorphic loci of the human CYP3A5 gene, wherein the presence of a variant allele at said one or more polymorphic loci indicates an increased likelihood of achieving a favorable response to a DPP-IV inhibitor relative to an individual harboring the reference allele at that locus.

3. The method according to claim 1 or 2, wherein said polymorphic locus is at nucleotide position 7068 of SEQ ID NO:1 or SEQ ID NO:2.

4. The method according to claim 3, wherein said reference allele at the polymorphic locus is “G”.

5. The method according to claim 3, wherein said variant allele at the polymorphic locus is “A”.

6. A method of identifying a subject who may benefit from the administration of a pharmaceutically acceptable amount of a DPP-IV inhibitor comprising the step of determining whether said subject has a reference or variant allele at one or more polymorphic loci of the human CYP3A5 gene, wherein the presence of a variant allele at said one or more polymorphic loci indicates an increased likelihood that said subject will benefit from the administration of said DPP-IV inhibitor relative to a subject harboring the reference allele at that locus.

7. The method of claim 1, 2, or 6, wherein the subject is of Hispanic descent.

8. The method according to 1, 2, 6, or 7, wherein said DPP-IV inhibitor is saxagliptin.

9. A method of identifying an individual who may have an increased likelihood of achieving a favorable response to the administration of a pharmaceutically acceptable amount of a DPP-IV inhibitor comprising the step of determining whether said individual has a reference or variant allele at one or more polymorphic loci of the human Insulin Promoter Factor-1 (IPF-1) gene, wherein the presence of a reference allele at said one or more polymorphic loci indicates a decreased likelihood of achieving a favorable response to a DPP-IV inhibitor relative to an individual harboring the variant allele at that locus.

10. A method of identifying an individual who may have an increased likelihood of achieving a favorable response to the administration of a pharmaceutically acceptable amount of a DPP-IV inhibitor comprising the step of determining whether said individual has a reference or variant allele at one or more polymorphic loci of the human IPF-1 gene, wherein the presence of a variant allele at said one or more polymorphic loci indicates an increased likelihood of achieving a favorable response to a DPP-IV inhibitor relative to an individual harboring the reference allele at that locus.

11. The method according to claim 9 or 10, wherein said polymorphic locus is at nucleotide position 4445 of SEQ ID NO:11 or SEQ ID NO:12.

12. The method according to claim 11, wherein said reference allele at the polymorphic locus is “C”.

13. The method according to claim 11, wherein said variant allele at the polymorphic locus is “T”.

14. A method of identifying a subject who may benefit from the administration of a pharmaceutically acceptable amount of a DPP-IV inhibitor comprising the step of determining whether said subject has a reference or variant allele at one or more polymorphic loci of the human IPF-1 gene, wherein the presence of a variant allele at said one or more polymorphic loci indicates an increased likelihood said individual will benefit from the administration of said DPP-IV inhibitor relative to a subject harboring the reference allele.

15. The method according to claim 9, 10, or 14, wherein said DPP-IV inhibitor is saxagliptin.
